(19) United States
(12) Patent Application Publication
(10) Pub. No.: US 2025/0367137 A1
(43) Pub. Date: Dec. 4, 2025

(54) Phloroglucinol Formulations And Methods Of Use

(71) Applicant: CinPhloro Pharma, LLC, Cincinnati, OH (US)

(72) Inventors: Mary BOND, Cincinnati, OH (US); Dalia LEVINE, Tel Aviv (IL); Brian MURPHY, Cincinnati, OH (US); Jon ISAACSOHN, Cincinnati, OH (US); Piyush PATEL, Downington, PA (US)

(21) Appl. No.: 18/871,882

(22) PCT Filed: Jun. 13, 2023
(86) PCT No.: PCT/US2023/025176

§ 371 Date: Dec. 5, 2024

Related U.S. Application Data
(60) Provisional application No. 63/351,572, filed on Jun. 13, 2022.

(51) Int. Cl. A61K 31/05; A61K 9/48; A61K 9/50

(52) U.S. Cl. CPC A61K 31/05; A61K 9/4866; A61K 9/501; A61K 9/5015; A61K 9/5026; A61K 9/5073

(57) ABSTRACT

The present disclosure provides pharmaceutical compositions comprising a total amount of about 50 mg to about 1000 mg of an active pharmaceutical ingredient (API) that is phloroglucinol, trimethylphloroglucinol, a pharmaceutically acceptable salt thereof, or a combination thereof. The pharmaceutical composition comprises an immediate release portion, a first modified release portion, and a second modified release portion. Also provided are oral dosage units comprising the pharmaceutical compositions and methods of treating a spasmodic condition in a subject in need thereof using the pharmaceutical compositions or oral dosage units.

### CROSS-REFERENCE TO RELATED APPLICATIONS

This application claims the benefit of the priority of U.S. Provisional Patent Application No. 63/351,572, filed Jun. 13, 2022, the disclosure of which is incorporated by reference herein.

### TECHNICAL FIELD

The disclosure relates to pharmaceutical compositions comprising active pharmaceutical ingredient (API) that is phloroglucinol, trimethylphloroglucinol, a pharmaceutically acceptable salt thereof, or a combination thereof and methods of using the same.

### BACKGROUND

Phloroglucinol is an antispasmodic, approved in France (as Spasfon™) and several other countries for relief of pain in gastrointestinal disorders. In placebo-controlled trials, phloroglucinol demonstrated statistically significant improvement in both pain and stool frequency for irritable bowel syndrome (IBS) sufferers. Despite its demonstrated efficacy, the PK profile of phloroglucinol does make it difficult to administer on a chronic, daily basis. Following administration of existing formulations, an extremely rapid maximum plasma concentration (Cₘₐₓ) is attained, followed by a rapid decline. As a result, frequent dosing (up to 6 times per day) is necessary.

Formulations are needed that maintain efficacy with less frequent dosing.

### SUMMARY

In some embodiments, the disclosure provides pharmaceutical compositions comprising a total amount of about 50 mg to about 1000 mg of an active pharmaceutical ingredient (API) that is phloroglucinol, trimethylphloroglucinol, a pharmaceutically acceptable salt thereof, or a combination thereof. The pharmaceutical composition comprises an immediate release portion comprising about 20-40% by weight of the total amount of the API, wherein at least about 90% by weight of the API in the immediate release portion is released from the pharmaceutical composition within about 2 hours, as measured by the USP 2 paddle method at about 50 rpm in about 750 mL of an aqueous solution comprising about 0.1N HCl solution at about 37° C. The pharmaceutical composition also comprises a first modified release portion comprising about 20-40% by weight of the total amount of the API, wherein at least about 90% by weight of the API in the first modified release portion is released from the pharmaceutical composition after at least about 2 hours to about 4 hours, as measured by the USP 2 paddle method at about 50 rpm in about 750 ml of an aqueous solution comprising about 0.1N HCl solution at about 37° C. The pharmaceutical composition further comprises a second modified release portion comprising about 20-40% by weight of the total amount of the API, wherein at least about 90% by weight of the API in the second modified release portion is released from the pharmaceutical composition after about 4 or more hours, as measured by the USP 2 paddle method at about 50 rpm in about 750 mL of an aqueous solution comprising about 0.1N HCl solution at about 37° C. An oral dosage unit comprising the pharmaceutical composition of any one of the preceding claims.

In other embodiments, the disclosure provides methods of treating a spasmodic condition in a subject in need thereof, comprising orally administering to the subject, an oral dosage unit described herein, (i) wherein the oral administration to the subject in a fed state results in a median Tₘₐₓ of the API of about 0.5 to about 1.75 hours; a mean Cₘₐₓ of the API of about 269 to about 1512 ng/ml; a mean AUCₜₐᵤ of the API of about 879 to about 4695 h*ng/ml; and/or a mean t½ of the API of about 1.6 hours to about 2.4 hours; or (ii) wherein the oral administration to the subject in a fasted state results in a median Tₘₐₓ of the API of about 0.5 hours; a mean Cₘₐₓ of the API of about 2745 to about 4874 ng/ml; a mean AUCₜₐᵤ of the API of about 4567 to about 6853 h*ng/ml; and/or a mean t½ of the API of about 2 hours.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific compositions, methods, devices, and systems disclosed. In addition, the drawings are not necessarily drawn to scale.

FIGS. 1A-1C are plots of mean (+SD) plasma phloroglucinol concentrations on Day 1 by dose level on a semi-logarithmic scale-PK population. In these figures, LLOQ for phloroglucinol=10 ng/ml; mean concentrations at any individual time point were only calculated if at least half of the subjects had valid values (i.e., quantifiable and not missing) at this time point for each treatment.

FIGS. 2A-2C are plots of mean (+SD) plasma trough phloroglucinol concentrations by dose level on a semi-logarithmic scale-PK population. In these figures, LLOQ for phloroglucinol=10 ng/ml. BID tau=10 h, only AM dose on Day 6. Mean concentrations at any individual time point were only calculated if at least half of the subjects had valid values (i.e., quantifiable and not missing) at this time point for each treatment.

FIG. 3 is a flow diagram of a manufacturing process to prepare the dosage forms of the disclosure.

FIG. 4 is the dissolution profile for the capsules of Example 1.

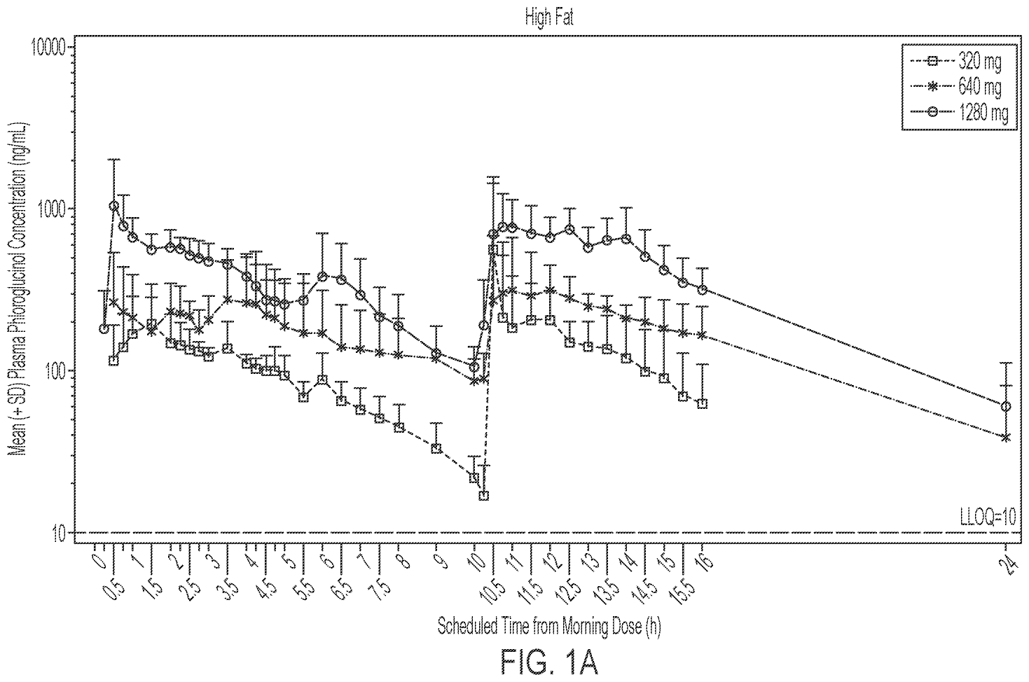

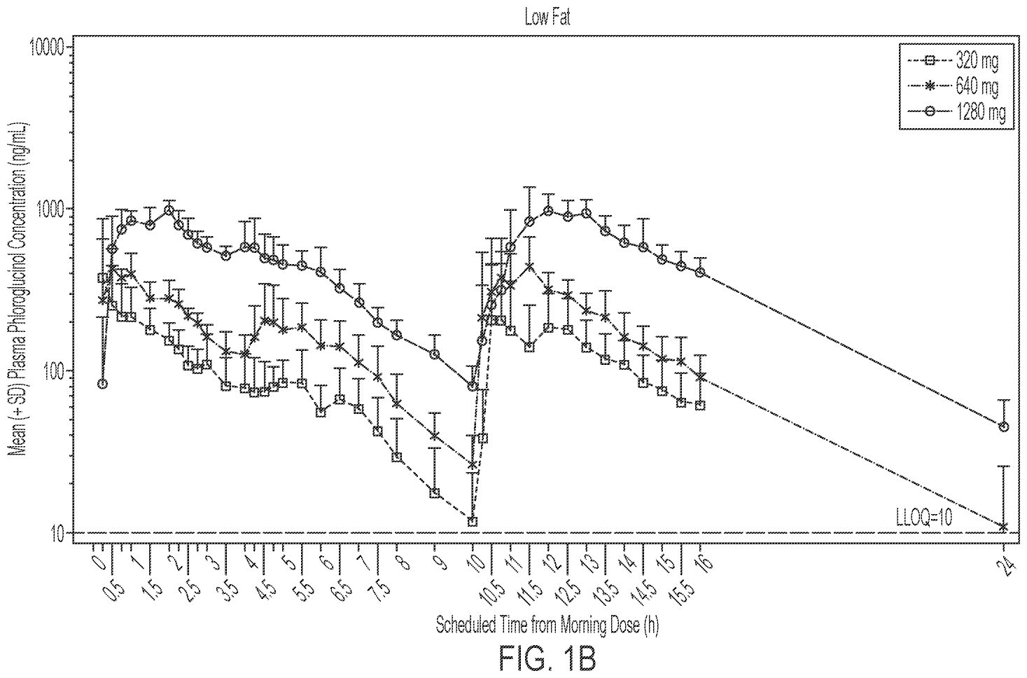

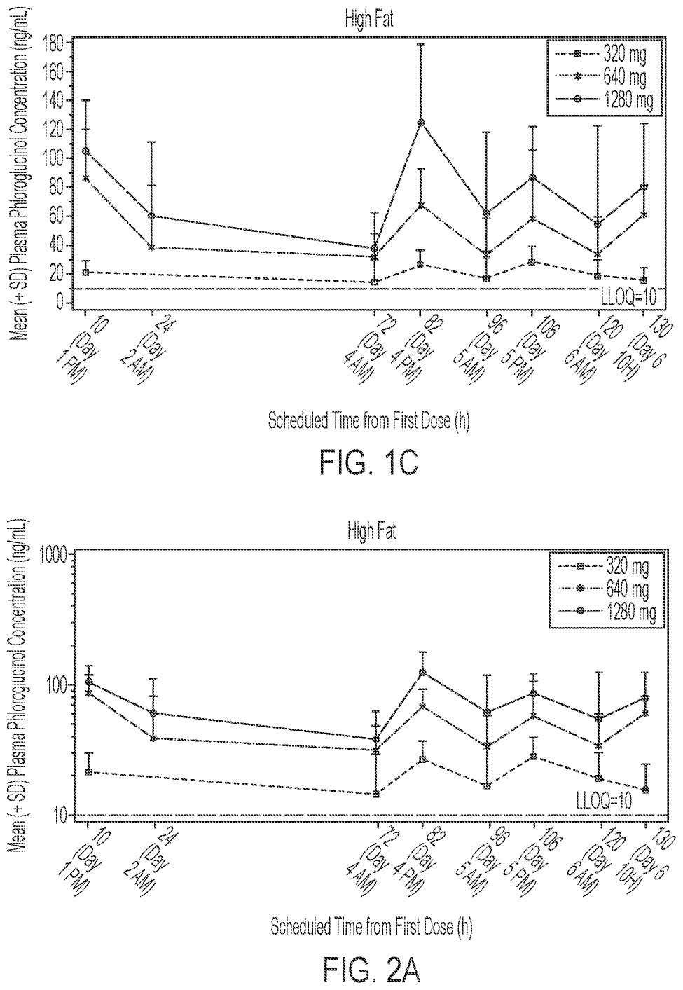

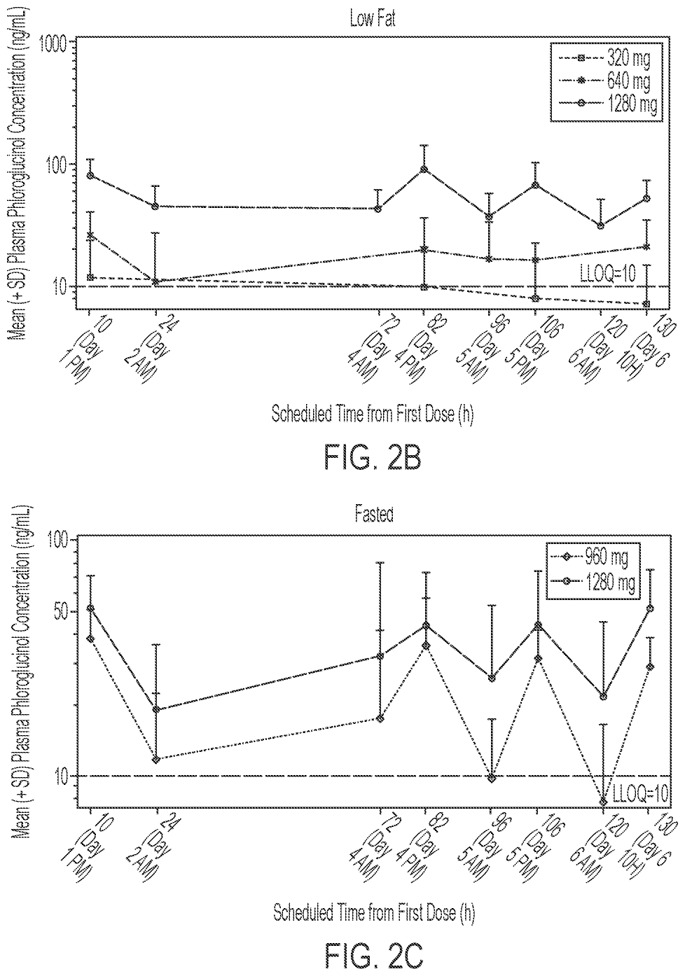

### DETAILED DESCRIPTION OF ILLUSTRATIVE EMBODIMENTS

The present disclosure provides pharmaceutical compositions that are capable of releasing an active pharmaceutical ingredient over predetermined periods of time. The compositions contain immediate release and delayed release portions, thereby providing a sustained release of the API. By doing so, patients do not need to continuously administer oral dosage forms of the API throughout the day. As such, the API is administered on a more regular basis and conditions are more reliably treated.

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

The modifier “about” should be considered as disclosing the range defined by the absolute values of the two endpoints. For example, the expression “from about 2 to about 4” also discloses the range “from 2 to 4.” When used to modify a single number, the term “about” may refer to plus or minus 10% of the indicated number and includes the indicated number. For example, “about 10%” may indicate a range of 9% to 11%, and “about 1” means from 0.9 to 1.1.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

The terms “patient” or “subject” as used herein refer to a mammalian animal and are used interchangeably. In some embodiments, the patient or subject is a human. In other embodiments, the patient or subject is a veterinary or farm animal, a domestic animal or pet, or animal normally used for clinical research.

“Treating” any disease or disorder refers, in some embodiments, to ameliorating a disease or disorder (i.e., arresting or reducing the development of the disease or at least one of the clinical symptoms thereof). The “treating” refers to ameliorating a disease or disorder using phloroglucinol, trimethylphloroglucinol, or a combination thereof. In some embodiments, “treating” or “treatment” refers to ameliorating at least one physical parameter, which may not be discernible by the subject. In other embodiments, “treating” or “treatment” refers to modulating the disease or disorder, either physically, (e.g., stabilization of a discernible symptom), physiologically, (e.g., stabilization of a physical parameter), or both. In further embodiments, “treating” or “treatment” refers to delaying the onset of the disease or disorder.

The present disclosure provides pharmaceutical compositions comprising an active pharmaceutical ingredient (API) that is phloroglucinol, trimethylphloroglucinol, a pharmaceutically acceptable salt thereof, or a combination thereof. In some embodiments, the API is phloroglucinol or a pharmaceutically acceptable salt thereof. In other embodiments, the API is trimethylphloroglucinol or a pharmaceutically acceptable salt thereof.

The term “phloroglucinol” as used herein refers to the following compound.

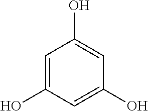

Phloroglucinol also includes any tautomeric forms thereof, including its known keto tautomer shown below.

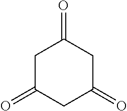

Similarly, the term “trimethylphloroglucinol” as used herein refers to the following compound.

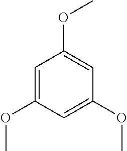

Phloroglucinol and trimethylphloroglucinol may be used in the form of salts derived from pharmaceutically or physiologically acceptable acids, bases, alkali metals and alkaline earth metals. In some embodiments, pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. In other embodiments, pharmaceutically acceptable salts may also be formed from inorganic bases, desirably alkali metal salts including, e.g., sodium, lithium, or potassium, such as alkali metal hydroxides. Examples of inorganic bases include, without limitation, sodium hydroxide, potassium hydroxide, calcium hydroxide, and magnesium hydroxide. Pharmaceutically acceptable salts may also be formed from organic bases, such as ammonium salts, mono-, di-, and trimethylammonium, mono-, di- and triethylammonium, mono-, di- and tripropylammonium, ethyldimethylammonium, benzyldimethylammonium, cyclohexylammonium, benzyl-ammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1 n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmono-ethanolammonium, diethanolamine, ethylenediamine, and the like. In one example, the base is sodium hydroxide, lithium hydroxide, potassium hydroxide, or mixtures thereof.

The pharmaceutical composition contains a total amount of about 50 mg to about 1000 mg of the API. In some embodiments, the pharmaceutical composition contains a total amount of about 50, about 100, about 150, about 200, about 250, about 300, about 350, about 400, about 450, about 500, about 550, about 600, about 650, about 700, about 750, about 800, about 850, about 900, about 950, or about 1000 mg of the API. In further embodiments, the pharmaceutical composition contains about 100 to about 1000, about 100 to about 900, about 100 to about 800, about 100 to about 700, about 100 to about 600, about 100 to about 500, about 100 to about 400, about 100 to about 300, about 100 to about 200, about 200 to about 1000, about 200 to about 900, about 200 to about 800, about 200 to about 700, about 200 to about 600, about 200 to about 500, about 200 to about 400, about 200 to about 300, about 300 to about 1000, about 300 to about 900, about 300 to about 800, about 300 to about 700, about 300 to about 600, about 300 to about 500, about 300 to about 400, about 400 to about 1000, about 400 to about 900, about 400 to about 800, about 400 to about 700, about 400 to about 600, about 400 to about 500, about 500 to about 1000, about 500 to about 900, about 500 to about 800, about 500 to about 700, about 500 to about 600, about 600 to about 1000, about 600 to about 900 m about 600 to about 800, about 600 to about 700, about 700 to about 1000, about 700 to about 900, about 700 to about 800, about 800 to about 1000, about 800 to about 900, or about 900 to about 1000 mg of the API.

The pharmaceutical composition comprises an immediate release portion, a first modified release portion, and a second modified release portion. In some embodiments, the immediate release portion and first modified release portion contain the same API. In other embodiments, the immediate release portion and second modified release portion contain the same API. In further embodiments, the first and second modified release portions contain the same API. In yet other embodiments, the immediate release, first modified release, and second modified release portions contain the same API.

The immediate release portion comprises about 20 to about 40% by weight of the API, based on the weight of the pharmaceutical composition. In some embodiments, the immediate release portion contains about 20, about 21, about 22, about 23, about 24, about 25, about 26, about 27, about 28, about 29, about 30, about 31, about 32, about 33, about 34, about 35, about 36, about 37, about 38, about 39, or about 40% by weight of the API, based on the weight of the pharmaceutical composition. In further embodiments, the immediate release portion contains about 20 to about 40, about 20 to about 38, about 20 to about 36, about 20 to about 35, about 20 to about 34, about 20 to about 32, about 20 to about 30, about 20 to about 28, about 20 to about 26, about 20 to about 25, about 20 to about 24, about 20 to about 22, about 22 to about 40, about 22 to about 38, about 22 to about 36, about 22 to about 35, about 22 to about 34, about 22 to about 32, about 22 to about 30, about 22 to about 28, about 22 to about 26, about 22 to about 24, about 24 to about 40, about 24 to about 38, about 24 to about 36, about 24 to about 35, about 24 to about 34, about 24 to about 32, about 24 to about 30, about 24 to about 28, about 24 to about 26, about 26 to about 40, about 26 to about 38, about 26 to about 36, about 26 to about 35, about 26 to about 34, about 26 to about 32, about 26 to about 30, about 26 to about 28, about 28 to about 40, about 28 to about 38, about 28 to about 36, about 28 to about 35, about 28 to about 34, about 28 to about 32, about 28 to about 30, about 30 to about 40, about 30 to about 38, about 30 to about 36, about 30 to about 35, about 30 to about 34, about 30 to about 32, about 32 to about 40, about 32 to about 38, about 32 to about 36, about 32 to about 34, about 34 to about 40, about 34 to about 38, about 34 to about 36, about 36 to about 40, about 36 to about 38, or about 38 to about 40% by weight of the API, based on the weight of the pharmaceutical composition.

The immediate release portion may contain about 50 mg to about 500 mg of the API. In some embodiments, the immediate release portion contains about 50, about 75, about 100, about 125, about 150, about 160, about 175, about 200, about 225, about 250, about 275, about 300, about 325, about 350, about 400, about 425, about 450, about 475, or about 500 mg of the API. In further embodiments, the immediate release portion contains about 50 to about 450, about 50 to about 400, about 50 to about 350, about 50 to about 300, about 50 to about 250, about 50 to about 200, about 50 to about 150, about 50 to about 100, about 100 to about 500, about 100 to about 450, about 100 to about 400, about 100 to about 350, about 100 to about 300, about 100 to about 250, about 100 to about 200, about 100 to about 150, about 150 to about 500, about 150 to about 450, about 150 to about 400, about 150 to about 350, about 150 to about 300 m about 150 to about 250, about 150 to about 200, about 200 to about 500, about 200 to about 450, about 200 to about 400, about 200 to about 350, about 200 to about 300 about 200 to about 250, about 250 to about 500, about 250 to about 450, about 250 to about 400, about 250 to about 350, about 250 to about 300, about 300 to about 500, about 300 to about 450, about 300 to about 400, about 300 to about 350, about 350 to about 500, about 350 to about 450, about 350 to about 400, about 400 to about 500, about 400 to about 450, or about 450 to about 500 mg of the API. In yet other embodiments, the immediate release portions contains about 50 mg to about 400 mg of the API. In still further embodiments, the immediate release portion contains about 160 mg of the API.

According to the disclosure, at least about 90% by weight of the API in the immediate release portion is released from the pharmaceutical composition within about 2 hours, e.g., from administration of the API, as measured by the USP 2 paddle method at about 50 rpm in about 750 mL of an aqueous solution comprising about 0.1N HCl solution at about 37° C. In some embodiments, about 90, about 91, about 92, about 93, about 94, about 95, about 96, about 97, about 98, about 99, or about 100% by weight of the API in the immediate release portion is released from the pharmaceutical composition within about 2 hours, e.g., from administration of the API, as measured by the USP 2 paddle method at about 50 rpm in about 750 mL of an aqueous solution comprising about 0.1N HCl solution at about 37° C. In further embodiments, about 90 to about 100, about 90 to about 99, about 90 to about 98, about 90 to about 97, about 90 to about 96, about 90 to about 95, about 90 to about 94, about 90 to about 93, about 90 to about 92, about 90 to about 91, about 91 to about 100, about 91 to about 99, about 91 to about 98, about 91 to about 97, about 91 to about 96, about 91 to about 95, about 91 to about 94, about 91 to about 93, about 91 to about 92, about 92 to about 100, about 92 to about 99, about 92 to about 98, about 92 to about 97, about 92 to about 96, about 92 to about 95, about 92 to about 94, about 92 to about 93, about 93 to about 100, about 93 to about 99, about 93 to about 98, about 93 to about 97, about 93 to about 96, about 93 to about 95, about 93 to about 94, about 94 to about 100, about 94 to about 99, about 94 to about 98, about 94 to about 97, about 94 to about 96, about 94 to about 95, about 95 to about 100, about 95 to about 99, about 95 to about 98, about 95 to about 97, about 95 to about 96, about 96 to about 100, about 96 to about 99, about 96 to about 98, about 96 to about 97, about 97 to about 100, about 97 to about 99, about 97 to about 98, about 98 to about 100, about 98 to about 99, or about 99 to about 100% by weight of the API in the immediate release portion is released from the pharmaceutical composition within about 2 hours, e.g., from administration of the API, as measured by the USP 2 paddle method at about 50 rpm in about 750 mL of an aqueous solution comprising about 0.1N HCl solution at about 37° C.

The term “within about 2 hours” refers to a timeframe from about 1 minute to about 2 hours. In certain aspects, the API is released within about 2 hours from administration of the API. In some embodiments, the API is released from the immediate release portion in about 1, about 10, about 20, about 30, about 40, about 50, about 60, about 70, about 80, about 90, about 100, about 110, or about 120 minutes, e.g., from administration of the API. In further embodiments, the API is released from the immediate release portion in about 1 to about 120 minutes, about 1 to about 110, about 1 to about 100, about 1 to about 90, about 1 to about 80, about 1 to about 70, about 1 to about 60, about 1 to about 50, about 1 to about 40, about 1 to about 30, about 1 to about 20, about 1 to about 10, about 10 to about 120, about 10 to about 110, about 10 to about 100, about 10 to about 90, about 10 to about 80, about 10 to about 70, about 10 to about 60, about 10 to about 50, about 10 to about 40, about 10 to about 30, about 10 to about 20, about 20 to about 120, about 20 to about 110, about 20 to about 100, about 20 to about 90, about 20 to about 80, about 20 to about 70, about 20 to about 60, about 20 to about 50, about 20 to about 40, about 20 to about 30, about 30 to about 120, about 30 to about 110, about 30 to about 100, about 30 to about 90, about 30 to about 80, about 30 to about 70, about 30 to about 60, about 30 to about 50, about 30 to about 40, about 40 to about 120, about 40 to about 110, about 40 to about 100, about 40 to about 90, about 40 to about 80, about 40 to about 70, about 40 to about 60, about 40 to about 50, about 50 to about 120, about 50 to about 110, about 50 to about 100, about 50 to about 90, about 50 to about 80, about 50 to about 70, about 50 to about 60, about 60 to about 120, about 60 to about 110, about 60 to about 100, about 60 to about 90, about 60 to about 80, about 60 to about 70, about 70 to about 120, about 70 to about 110, about 70 to about 100, about 70 to about 90, about 70 to about 80, about 80 to about 120, about 80 to about 110, about 80 to about 100, about 80 to about 90, about 90 to about 120, about 90 to about 110, about 90 to about 100, about 100 to about 120, about 100 to about 110, or about 110 to about 120 minutes, e.g., from administration of the API.

According to the disclosure, the first modified release portion comprises about 20 to about 40% by weight of the API, based on the weight of the pharmaceutical composition. In some embodiments, the first modified release portion contains about 20, about 21, about 22, about 23, about 24, about 25, about 26, about 27, about 28, about 29, about 30, about 31, about 32, about 33, about 34, about 35, about 36, about 37, about 38, about 39, or about 40% by weight of the API, based on the weight of the pharmaceutical composition. In further embodiments, the first modified release portion contains about 20 to about 40, about 20 to about 38, about 20 to about 36, about 20 to about 35, about 20 to about 34, about 20 to about 32, about 20 to about 30, about 20 to about 28, about 20 to about 26, about 20 to about 25, about 20 to about 24, about 20 to about 22, about 22 to about 40, about 22 to about 38, about 22 to about 36, about 22 to about 34, about 22 to about 32, about 22 to about 30, about 22 to about 38, about 22 to about 36, about 22 to about 34, about 22 to about 32, about 22 to about 30, about 22 to about 28, about 22 to about 26, about 22 to about 24, about 24 to about 40, about 24 to about 38, about 24 to about 36, about 24 to about 34, about 24 to about 32, about 24 to about 30, about 24 to about 28, about 24 to about 26, about 26 to about 40, about 26 to about 38, about 26 to about 36, about 26 to about 34, about 26 to about 32, about 26 to about 30, about 26 to about 28, about 28 to about 40, about 28 to about 38, about 28 to about 36, about 28 to about 34, about 28 to about 32, about 28 to about 30, about 30 to about 40, about 30 to about 38, about 30 to about 36, about 30 to about 34, about 30 to about 32, about 32 to about 40, about 32 to about 38, about 32 to about 36, about 32 to about 34, about 34 to about 40, about 34 to about 38, about 34 to about 36, about 36 to about 40, about 36 to about 38, or about 38 to about 40% by weight of the API, based on the weight of the pharmaceutical composition.

The first modified release portion may contain about 25 mg to about 200 mg of the API. In some embodiments, the first modified release portion contains about 25, about 50, about 75, about 80, about 100, about 125, about 150, about 160, about 175, or about 200 mg of the API. In other embodiments, the first modified release portion contains about 25 to about 200, about 25 to about 175, about 25 to about 160, about 25 to about 150, about 25 to about 125, about 25 to about 100, about 25 to about 75, about 25 to about 60, about 25 to about 50, about 50 to about 200, about 50 to about 175, about 50 to about 160, about 50 to about 150, about 50 to about 125, about 50 to about 100, about 50 to about 80, about 50 to about 75, about 75 to about 200, about 75 to about 175, about 75 to about 160, about 75 to about 150, about 75 to about 125, about 75 to about 100, about 75 to about 80, about 80 to about 200, about 80 to about 175, about 80 to about 160, about 80 to about 150, about 80 to about 125, about 80 to about 100, about 100 to about 200, about 100 to about 175, about 100 to about 160, about 100 to about 150, about 100 to about 125, about 125 to about 200, about 125 to about 175, about 125 to about 160, about 125 to about 150, about 150 to about 200, about 150 to about 175, about 150 to about 160, about 160 to about 200, about 160 to about 175, or about 175 to about 200 mg of the API. In further embodiments, the first modified release portion contains about 50 mg to about 200 mg of the API. In yet other embodiments, the first modified release portion contains about 160 mg of the API. In still further embodiments, the first modified release portion contains about 80 mg of the API.

At least about 90% by weight of the API in the first modified release portion is released from the pharmaceutical composition after at least about 2 hours to about 4 hours, e.g., from administration of the API, as measured by the USP 2 paddle method at about 50 rpm in about 750 mL of an aqueous solution comprising about 0.1N HCl solution at about 37° C. In some embodiments, about 90, about 91, about 92, about 93, about 94, about 95, about 96, about 97, about 98, about 99, or about 100% by weight of the API in the first modified release portion is released from the pharmaceutical composition after at least about 2 hours to about 4 hours, e.g., about 2, about 2.25, about 2.5, about 2.75, about 3, about 3.25, about 3.5, about 3.75, or about 4 hours, or such as about 2 to about 3.75, about 2 to about 3.5, about 2 to about 3.25, about 2 to about 3, about 2 to about 2.75, about 2 to about 2.5, about 2 to about 2.25, about 2.25 to about 4, about 2.25 to about 3.75, about 2.25 to about 3.5, about 2.25 to about 3.25, about 2.25 to about 3, about 2.25 to about 2.75, about 2.25 to about 2.5, about 2.5 to about 4, about 2.5 to about 3.75, about 2.5 to about 3.5, about 2.5 to about 3.25, about 2.5 to about 3, about 2.5 to about 2.75, about 2.75 to about 4, about 2.75 to about 3.75, about 2.75 to about 3.5, about 2.75 to about 3.25, about 2.75 to about 3, about 3 to about 4, about 3 to about 3.75, about 3 to about 3.5, about 3 to about 3.25, about 3.25 to about 4, about 3.25 to about 3.75, about 3.25 to about 3.5, about 3.5 to about 4, about 3.5 to about 3.75, or about 3.75 to about 4, e.g., from administration of the API. In further embodiments, about 90 to about 100, about 90 to about 99, about 90 to about 98, about 90 to about 97, about 90 to about 96, about 90 to about 95, about 90 to about 94, about 90 to about 93, about 90 to about 92, about 90 to about 91, about 91 to about 100, about 91 to about 99, about 91 to about 98, about 91 to about 97, about 91 to about 96, about 91 to about 95, about 91 to about 94, about 91 to about 93, about 91 to about 92, about 92 to about 100, about 92 to about 99, about 92 to about 98, about 92 to about 97, about 92 to about 96, about 92 to about 95, about 92 to about 94, about 92 to about 93, about 93 to about 100, about 93 to about 99, about 93 to about 98, about 93 to about 97, about 93 to about 96, about 93 to about 95, about 93 to about 94, about 94 to about 100, about 94 to about 99, about 94 to about 98, about 94 to about 97, about 94 to about 96, about 94 to about 95, about 95 to about 100, about 95 to about 99, about 95 to about 98, about 95 to about 97, about 95 to about 96, about 96 to about 100, about 96 to about 99, about 96 to about 98, about 96 to about 97, about 97 to about 100, about 97 to about 99, about 97 to about 98, about 98 to about 100, about 98 to about 99, or about 99 to about 100% by weight of the API in the first modified release portion is released from the pharmaceutical composition after at least about 2 hours to about 4 hours, e.g., from administration of the API, as measured by the USP 2 paddle method at about 50 rpm in about 750 mL of an aqueous solution comprising about 0.1N HCl solution at about 37° C.

According to the disclosure, the second modified release portion comprises about 20 to about 40% by weight of the API, based on the weight of the pharmaceutical composition. In some embodiments, the second modified release portion contains about 20, about 21, about 22, about 23, about 24, about 25, about 26, about 27, about 28, about 29, about 30, about 31, about 32, about 33, about 34, about 35, about 36, about 37, about 38, about 39, or about 40% by weight of the API, based on the weight of the pharmaceutical composition. In further embodiments, the second modified release portion contains about 20 to about 40, about 20 to about 38, about 20 to about 36, about 20 to about 35, about 20 to about 34, about 20 to about 32, about 20 to about 30, about 20 to about 28, about 20 to about 26, about 20 to about 25, about 20 to about 24, about 20 to about 22, about 22 to about 40, about 22 to about 38, about 22 to about 36, about 22 to about 35, about 22 to about 34, about 22 to about 32, about 22 to about 30, about 22 to about 38, about 22 to about 36, about 22 to about 34, about 22 to about 32, about 22 to about 30, about 22 to about 28, about 22 to about 26, about 22 to about 24, about 24 to about 40, about 24 to about 38, about 24 to about 36, about 24 to about 35, about 24 to about 34, about 24 to about 32, about 24 to about 30, about 24 to about 28, about 24 to about 26, about 26 to about 40, about 26 to about 38, about 26 to about 36, about 26 to about 35, about 26 to about 34, about 26 to about 32, about 26 to about 30, about 26 to about 28, about 28 to about 40, about 28 to about 38, about 28 to about 36, about 28 to about 35, about 28 to about 34, about 28 to about 32, about 28 to about 30, about 30 to about 40, about 30 to about 38, about 30 to about 36, about 30 to about 35, about 30 to about 34, about 30 to about 32, about 32 to about 40, about 32 to about 38, about 32 to about 36, about 32 to about 35, about 32 to about 34, about 34 to about 40, about 34 to about 38, about 34 to about 36, about 34 to about 35, about 35 to about 40, about 35 to about 36, about 36 to about 40, about 36 to about 38, or about 38 to about 40% by weight of the API, based on the weight of the pharmaceutical composition.

The second modified release portion may contain about 25 mg to about 200 mg of the API. In some embodiments, the second modified release portion contains about 25, about 50, about 75, about 80, about 100, about 125, about 150, about 160, about 175, or about 200 mg of the API. In other embodiments, the second modified release portion contains about 25 to about 200, about 25 to about 175, about 25 to about 160, about 25 to about 150, about 25 to about 125, about 25 to about 100, about 25 to about 75, about 25 to about 60, about 25 to about 50, about 50 to about 200, about 50 to about 175, about 50 to about 160, about 50 to about 150, about 50 to about 125, about 50 to about 100, about 50 to about 80, about 50 to about 75, about 75 to about 200, about 75 to about 175, about 75 to about 160, about 75 to about 150, about 75 to about 125, about 75 to about 100, about 75 to about 80, about 80 to about 200, about 80 to about 175, about 80 to about 160, about 80 to about 150, about 80 to about 125, about 80 to about 100, about 100 to about 200, about 100 to about 175, about 100 to about 160, about 100 to about 150, about 100 to about 125, about 125 to about 200, about 125 to about 175, about 125 to about 160, about 125 to about 150, about 150 to about 200, about 150 to about 175, about 150 to about 160, about 160 to about 200, about 160 to about 175, or about 175 to about 200 mg of the API. In further embodiments, the second modified release portion contains about 50 mg to about 200 mg of the API. In yet other embodiments, the second modified release portion contains about 160 mg of the API. In still further embodiments, the second modified release portion contains about 80 mg of the API.

At least about 90% by weight of the API in the second modified release portion is released from the pharmaceutical composition after 4 or more hours, e.g., from administration of the API, as measured by the USP 2 paddle method at about 50 rpm in about 750 mL of an aqueous solution comprising about 0.1N HCl solution at about 37° C. In some embodiments, about 90, about 91, about 92, about 93, about 94, about 95, about 96, about 97, about 98, about 99, or about 100% by weight of the API in the second modified release portion is released from the pharmaceutical composition after about 4 or more hours, e.g., from administration of the API, e.g., about 4, about 5, about 6, about 7, about 8, about 9, about 10, about 11, or about 12 hours, or such as about 4 to about 12, about 4 to about 11, about 4 to about 10, about 4 to about 9, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 hours, e.g., from administration of the API. In further embodiments, about 90 to about 100, about 90 to about 99, about 90 to about 98, about 90 to about 97, about 90 to about 96, about 90 to about 95, about 90 to about 94, about 90 to about 93, about 90 to about 92, about 90 to about 91, about 91 to about 100, about 91 to about 99, about 91 to about 98, about 91 to about 97, about 91 to about 96, about 91 to about 95, about 91 to about 94, about 91 to about 93, about 91 to about 92, about 92 to about 100, about 92 to about 99, about 92 to about 98, about 92 to about 97, about 92 to about 96, about 92 to about 95, about 92 to about 94, about 92 to about 93, about 93 to about 100, about 93 to about 99, about 93 to about 98, about 93 to about 97, about 93 to about 96, about 93 to about 95, about 93 to about 94, about 94 to about 100, about 94 to about 99, about 94 to about 98, about 94 to about 97, about 94 to about 96, about 94 to about 95, about 95 to about 100, about 95 to about 99, about 95 to about 98, about 95 to about 97, about 95 to about 96, about 96 to about 100, about 96 to about 99, about 96 to about 98, about 96 to about 97, about 97 to about 100, about 97 to about 99, about 97 to about 98, about 98 to about 100, about 98 to about 99, or about 99 to about 100% by weight of the API in the second modified release portion is released from the pharmaceutical composition after about 4 or more hours, e.g., from administration of the API, as measured by the USP 2 paddle method at about 50 rpm in about 750 mL of an aqueous solution comprising about 0.1N HCl solution at about 37° C.

As used herein “release of the API” from the immediate release portion, first modified release portion, second modified release portion, or a combination thereof is measured by the USP 2 paddle method at about 50 rpm in about 750 mL of an aqueous solution comprising about 0.1N HCl solution at about 37° C.

### Dosage Units

As discussed herein, the present disclosure provides dosage units that contain the pharmaceutical compositions described herein. In some embodiments, the oral dosage units are formulated for oral administration, i.e., are oral dosage units. The oral dosage unit may take a variety of delivery forms. In some embodiments, the dosage unit is a tablet, capsule (hard or soft), sachet, soft gel, liquid, gel, strip, film, or tablet-in-capsule. In other embodiments, the dosage unit is a tablet, capsule, or sachet. In further embodiments, the oral dosage unit is a tablet. In other embodiments, the oral dosage unit is a capsule. In yet further embodiments, the oral dosage unit is a sachet.

The term “tablet” as used herein refers to a solid dosage unit. The tablet may be of any shape or size convenient for oral administration, e.g., circular, elliptical, etc. Depending on the base of the tablet, it may be coated with a layer comprising the immediate release portion or modified release portion. In some embodiments, tablet is a bilayer tablet containing immediate release (IR) and modified release layers adjacent to each other. In other embodiments, the tablet is a trilayer tablet containing immediate release and modified release layers separated by a layer, for example, a buffer layer. In further embodiments, the tablet contains embedded within the tablet, granules coated with the immediate release portion and beads coated with the first modified release portion and/or the second modified release portion. In yet other embodiments, the tablet contains a tablet comprising the first modified release portion and/or the second modified release portion embedded within a tablet comprising the immediate release portion. In still further embodiments, the tablet contains a tablet comprising a first modified release portion and/or the second modified release portion that is suspended in a liquid solution comprising the immediate release portion, wherein the liquid solution is contained within a capsule. In other embodiments, a capsule of the disclosure contains a solution comprising the immediate release portion and coated, beads or granules coated with a first modified release portion and/or the second modified release portion. In further embodiments, a softgel of the disclosure contains a solution comprising the immediate release portion and beads or granules are coated with the first modified release portion and/or the second modified release portion.

The term “capsule” as used herein refers to a solid dosage unit. The capsule is typically elliptical in shape, but can adopt other forms, as determined by those skilled in the art. The capsule may be a hard or soft gelatin capsule, as needed. In some embodiments, the capsule contains a tablet comprising the immediate release portion and a tablet comprising the first modified release portion and/or the second modified release portion. In further embodiments, the capsule contains an immediate release tablet, a plug, and a modified release tablet including the first modified release portion and/or the second modified release portion. In other embodiments, the capsule contains beads coated with an immediate release portion and beads coated with a first modified release portion and/or the second modified release portion. In further embodiments, the capsule contains immediate release mini-tablets and modified release mini-tablets including the first modified release portion and/or the second modified release portion. In still other embodiments, the capsule contains immediate release granules and the granules are coated with a first modified release portion and/or the second modified release portion. In yet other embodiments, the capsule contains a plurality of beads coated with a first modified release and/or the second modified release portion and immediate release portions as layers.

The term “sachet” as used herein refers to a package that contains a mixture of immediate release and modified release granules or beads comprising the immediate release portion and granules or beads comprising the first modified release portion and/or the second modified release portion. The package may be selected by those skilled in the art.

Regardless of the form of the dosage unit, it may alternatively or in addition contain beads, granules, or a combination thereof. As used herein, the “beads” are solid particles that are prepared by extrusion and spheronization of the immediate release portion, first modified release portion, and/or the second modified release portion, or a combination thereof. In some embodiments, the pharmaceutical compositions are in the form of a plurality of beads. In other embodiments, the pharmaceutical compositions are in the form of a plurality of granules. The beads or granules contain a core and optional layers thereon the core. Similarly, the “granules” are solid particles, but they are prepared via a granulation. One of skill in the art would be able to select a suitable granulation method to prepare the granules for use herein. In some embodiments, the granulation method includes high-shear granulation, melt granulation, dry granulation, or wet granulation, among others. In some embodiments, the dosage unit contains beads comprising the immediate release portion. In other embodiments, the dosage unit contains beads comprising the first modified release portion. In further embodiments, the dosage unit contains beads comprising the second modified release portion. In yet other embodiments, dosage unit contains beads comprising the immediate release portion and the first modified release portion. In other embodiments, the dosage unit contains beads comprising the first modified release portion and the second modified release portion. In further embodiments, the dosage unit contains beads comprising the immediate release portion, first modified release portion and the second modified release portion.

Regardless of the particular presentation, the dosage form contains about 10 to about 60% by weight, based on the weight of the oral dosage unit, of the immediate release portion. In some embodiments, the dosage form contains about 10, about 15, about 20, about 25, about 30, about 35, about 40, about 41, about 42, about 43, about 44, about 45, about 46, about 47, about 48, about 49, about 50, about 55, or about 60% by weight, based on the weight of the oral dosage unit, of the immediate release portion. In further embodiments, the dosage form contains about 10 to about 55, about 10 to about 50, about 10 to about 45, about 10 to about 40, about 10 to about 35, about 10 to about 30, about 10 to about 25, about 10 to about 20, about 10 to about 15, about 15 to about 60, about 15 to about 55, about 15 to about 50, about 15 to about 45, about 15 to about 40, about 15 to about 35, about 15 to about 30, about 15 to about 25, about 15 to about 20, about 20 to about 60, about 20 to about 55, about 20 to about 50, about 20 to about 45, about 20 to about 40, about 20 to about 35, about 20 to about 30, about 20 to about 25, about 25 to about 60, about 25 to about 55, about 25 to about 50, about 25 to about 45, about 25 to about 40, about 25 to about 35, about 25 to about 30, about 30 to about 60, about 30 to about 55, about 30 to about 50, about 30 to about 45, about 30 to about 40, about 30 to about 35, about 35 to about 60, about 35 to about 55, about 35 to about 50 m, about 35 to about 45, about 35 to about 40, about 40 to about 60, about 40 to about 55, about 40 to about 50, about 40 to about 45, about 45 to about 60, about 45 to about 55, about 45 to about 50, about 50 to about 60, about 50 to about 55, or about 55 to about 60% by weight, based on the weight of the oral dosage unit, of the immediate release portion. In other embodiments, the dosage form contains about 30 to about 50% by weight, based on the weight of the oral dosage unit, of the immediate release portion. In still further embodiments, the dosage form contains about 43% by weight, based on the weight of the oral dosage unit, of the immediate release portion.

The dosage form also may contain about 10 to about 40% by weight, based on the weight of the oral dosage unit, of the first modified release portion. In some embodiments, the dosage form contains about 10, about 15, about 20, about 25, about 26, about 27, about 28, about 29, about 30, about 31, about 32, about 33, about 34, about 35, or about 40% by weight, based on the weight of the oral dosage unit, of the first modified release portion. In further embodiments, the dosage form contains about 10 about 40, about 10 to about 35, about 10 to about 30, about 10 to about 25, about 10 to about 20, about 10 to about 15, about 15 to about 40, about 15 to about 35, about 15 to about 30, about 15 to about 25, about 15 to about 20, about 20 to about 40, about 20 to about 35, about 20 to about 30, about 20 to about 25, about 25 to about 40, about 25 to about 35, about 25 to about 30, about 30 to about 40, about 30 to about 35, or about 35 to about 40% by weight, based on the weight of the oral dosage unit, of the first modified release portion. In other embodiments, the dosage form contains about 20 to about 30% by weight, based on the weight of the oral dosage unit, of the first modified release portion. In yet further embodiments, the dosage form contains about 28% by weight, based on the weight of the oral dosage unit, of the first modified release portion.

The dosage form also further contain about 10 to about 40% by weight, based on the weight of the oral dosage unit, of the second modified release portion. In some embodiments, the dosage form contains about 10, about 15, about 20, about 25, about 26, about 27, about 28, about 29, about 30, about 31, about 32, about 33, about 34, about 35, or about 40% by weight, based on the weight of the oral dosage unit, of the second modified release portion. In further embodiments, the dosage form contains about 10 to about 35, about 10 to about 30, about 10 to about 25, about 10 to about 20, about 10 to about 15, about 15 to about 40, about 15 to about 35, about 15 to about 30, about 15 to about 25, about 15 to about 20, about 20 to about 40, about 20 to about 35, about 20 to about 30, about 20 to about 25, about 25 to about 40, about 25 to about 35, about 25 to about 30, about 30 to about 40, about 30 to about 35, or about 35 to about 40% by weight, based on the weight of the oral dosage unit, of the second modified release portion. In other embodiments, the dosage form contains about 20 to about 30% by weight, based on the weight of the oral dosage unit, of the second modified release portion. In yet further embodiments, the dosage form contains about 28% by weight, based on the weight of the oral dosage unit, of the second modified release portion.

Typically, a plurality of beads or granules is incorporated into the dosage unit described herein. The term “plurality” as used herein refers to a number of beads or granules that provide the amount of phloroglucinol, trimethylphloroglucinol, or pharmaceutically acceptable salt required by the dosage unit. In some embodiments, the dosage unit comprises a plurality of beads. In further embodiments, the dosage unit comprises a plurality of granules. In other embodiments, the dosage unit comprises a plurality of beads and a plurality of granules.

The beads and/or granules contain one or both of the immediate release or modified release portions. In some embodiments, the beads comprise the immediate release portion. In other embodiments, the beads comprise the modified release portion. In further embodiments, the beads comprise the immediate release and modified release portions. In yet other embodiments, the granules comprise the immediate release portion. In still further embodiments, the granules comprise the modified release portion. In other embodiments, the granules comprise the immediate release and modified release portions.

The dosage forms or pharmaceutical compositions described herein may contain one or more different types of beads or granules. In certain aspects, the dosage form contains three type of beads or granules. For example, the dosage form or pharmaceutical compositions described herein contain (i) an immediate release portion comprising beads or granules containing the immediate release portion, (b) a first modified release portion comprising beads or granules containing the first modified release portion, (c) and a second modified release portion comprising beads or granules containing the second modified release portion. In other aspects, the dosage forms or pharmaceutical compositions described herein contain two types of beads or granules. In some embodiments, the dosage forms or pharmaceutical compositions described herein contain beads or granules comprising an immediate release portion coated onto a first modified release portion. In other embodiments, the dosage forms or pharmaceutical compositions described herein contain beads or granules comprising an immediate release portion coated onto a second modified release portion. In further embodiments, the dosage forms or pharmaceutical compositions described herein contain beads or granules comprising an immediate release portion coated onto a combination of a first release portion and a second release portion. In further aspects, the dosage forms or pharmaceutical compositions described herein contain one type of beads or granules. In some embodiments, the dosage forms or pharmaceutical compositions described herein contain beads or granules comprising a first modified release portion coated onto the second release portion. In other embodiments, the dosage forms or pharmaceutical compositions described herein contain beads or granules comprising a first modified release portion is coated onto the second release portion and an immediate release portion coated onto the first modified release portion.

Any of the beads or granules may be coated with a topcoat. In some embodiments, the beads or granules may be coated with a topcoat that is an enteric polymer that modulates the release of the API. For example, the first modified release portion comprises an API-containing core coated with a first enteric polymer capable of dissolution at a pH of about 5.5. The first enteric polymer may be selected by one skilled in the art. In some embodiment, the first enteric polymer is a methacrylic acid copolymer, such as a methacrylic acid ethyl acrylate copolymer, or such as a 1:1 methacrylic acid:ethyl acrylate copolymer, or such as a Eudragit® L 30 D-55 polymer. In some embodiment, the first enteric polymer is a methacrylic acid ethyl acrylate copolymer, hydroxypropyl methylcellulose acetate succinate, or cellulose acetate phthalate. In other embodiments, the first enteric polymer is a 1:1 methacrylic acid:ethyl acrylate copolymer. In further embodiments, the first enteric polymer is a Eudragit® L 30 D-55 polymer. In yet other embodiments, the API-containing core coated with the first enteric polymer is in the form of a bead or granule.

In other examples, the second modified release portion comprises an API-containing core coated with a second enteric polymer capable of dissolution at a pH of 6.8 or higher. In some embodiments, the second enteric polymer is a methacrylic acid copolymer, methylacrylate, or methyl methacrylate copolymer. In other embodiments, the second enteric polymer is a methacrylic acid. In further embodiments, the enteric polymer is methyl acrylate. In yet other embodiments, the second enteric polymer is a methyl methacrylate copolymer. In still further embodiments, the second enteric polymer is a Eudragit® FS 30D copolymer. In other embodiments, the API-containing core coated with the second enteric polymer is in the form of a bead or granule.

The dosage forms described herein desirably release all of the API over a prescribed period of time. In certain aspects, at least about 30% by weight of the total amount of API in the composition is released from the composition over a period of about 5 minutes to about 2 hours, e.g., from administration of the API. For example, about 30 to about 60% by weight of the total amount of API in the composition is released from the composition over a period of about 5 minutes to about 2 hours, e.g., from administration of the API. In some embodiments, about 30, about 35, about 40, about 45, about 50, about 55, or about 60% by weight of the total amount of API in the composition is released from the composition over a period of about 5 minutes to about 2 hour, e.g., from administration of the API. In further embodiments, about 30 to about 60, about 30 to about 55, about 30 to about 50, about 30 to about 45, about 30 to about 40, about 30 to about 35, about 35 to about 60, about 35 to about 55, about 35 to about 50, about 35 to about 45, about 35 to about 40, about 40 to about 60, about 40 to about 55, about 40 to about 50, about 40 to about 45, about 45 to about 60, about 45 to about 55, about 45 to about 50, about 50 to about 60, about 50 to about 55, or about 55 to about 60% by weight of the total amount of API in the composition is released from the composition over a period of about 5 minutes to about 2 hours, e.g., from administration of the API.

In other aspects, at least about 60% by weight of the total amount of the API in the composition is released from the pharmaceutical composition over a period of about 2 hours to about 4 hours, e.g., from administration of the API. For example, about 60 to about 80% by weight of the total amount of API in the composition is released from the composition over a period of about 2 hours to 4 hours, e.g., from administration of the API. In some embodiments, about 60, about 65, about 70, about 75, or about 80% by weight of the total amount of API in the composition is released from the composition over a period of about 2 hours to 4 hours, e.g., from administration of the API. In other embodiments, about 60 to about 75, about 60 to about 70, about 65 to 80, about 65 to about 75, about 65 to about 70, about 70 to about 80, about 70 to about 75, or about 75 to about 80% by weight of the total amount of API in the composition is released from the composition over a period of about 2 hours to 4 hours, e.g., from administration of the API.

In further aspects, at least about 80% by weight of the total amount of the API in the composition is released from the pharmaceutical composition over a period of about 3 hours to about 6 hours, e.g., from administration of the API. For example, about 80 to about 100% by weight of the total amount of the API in the composition is released from the pharmaceutical composition over a period of about 3 hours to about 6 hours, e.g., from administration of the API. In some embodiments, about 80, about 85, about 90, about 95, or about 100% by weight of the total amount of the API in the composition is released from the pharmaceutical composition over a period of about 3 hours to about 6 hours, e.g., from administration of the API. In further embodiments, about 80 to about 95, about 80 to about 90, about 80 to about 85, about 85 to about 100, about 85 to about 95, about 85 to about 90, about 90 to about 100, or about 95 to about 100 by weight of the total amount of the API in the composition is released from the pharmaceutical composition over a period of about 3 hours to about 6 hours, e.g., from administration of the API.

As used herein, “release of the API” is measured as measured by the USP 2 paddle method at about 50 rpm in about 750 mL of an aqueous solution comprising about 0.1N HCl solution at about 37° C.

The immediate release portion, first modified release portion, or second modified release portion or a combination thereof also contains an inactive pharmaceutical agent such as an excipient as described herein. In some embodiments, one or more of the immediate release portion, first modified release portion, or second modified release portion further comprises one or more of a filler, binder, disintegrant, emulsifier, anti-static agent, or solvent.

The beads and/or granules of the disclosure can have a diameter of about 50 to about 1500 μm. In some embodiments, the cores have a diameter of about 50 to about 1300 μm, about 50 to about 1100 μm, about 50 to about 900 μm, about 50 to about 800 μm, about 50 to about 700 μm, about 50 to about 600 μm, about 50 to about 500 μm, about 50 to about 400 μm, about 50 to about 300 μm, about 50 to about 200 μm, about 100 to about 1500 μm, about 100 to about 1300 μm, about 100 to about 1100 μm, about 100 to about 900 μm, about 100 to about 800 μm, about 100 to about 700 μm, about 100 to about 600 μm, about 100 to about 500 μm, about 100 to about 400 μm, about 100 to about 300 μm, about 100 to about 200 μm. In other embodiments, the core diameter is about 100 to about 800 μm.

The dosage unit may have multiple cores containing API with varying dissolution properties. Thus, the cores may be coated one or more layers. In some embodiments, the cores are coated with two or more layers, i.e., a multilayer tablet. In further embodiments, the cores are coated with an immediate release layer. In other embodiments, the cores are coated with a modified release layer. In yet further embodiments, the core is coated with an immediate release layer and coated with a modified release layer.

Other layers may be applied as a topcoat or in between the other layers. The layers may contain pharmaceutically inert components, i.e., as a buffer layer, or pharmaceutically active components, as determined by those skilled in the art.

The oral dosage units comprise one or more of phloroglucinol, trimethylphloroglucinol, or a pharmaceutically acceptable salt of phloroglucinol or trimethylphloroglucinol. In some embodiments, the oral dosage unit comprises phloroglucinol or a pharmaceutically acceptable salt thereof. In other embodiments, the oral dosage unit comprises trimethylphloroglucinol or a pharmaceutically acceptable salt thereof. In further embodiments, the oral dosage unit comprises phloroglucinol or a pharmaceutically acceptable salt thereof and trimethylphloroglucinol or a pharmaceutically acceptable salt thereof.

### Methods of Treatment

The pharmaceutical compositions and dosage units and formulations described herein are useful in treating spasmodic conditions in a subject. The methods comprise administering a pharmaceutical composition or an oral dosage unit described herein to the subject. In some embodiments, the spasmodic condition is a sudden involuntary muscle contraction of a body part, such as an organ or muscle, of the subject. In other embodiments, the spasmodic condition is a sudden involuntary muscle contraction of the bronchi, stomach, intestine, ureter, gall bladder, kidney, or bile duct. In further embodiments, the spasmodic condition is a sudden involuntary muscle contraction of the bronchi. In yet other embodiments, sudden involuntary muscle contraction of the stomach. In still further embodiments, the sudden involuntary muscle contraction of the intestine. In other embodiments, the sudden involuntary muscle contraction of the ureter. In further embodiments, the sudden involuntary muscle contraction of the gall bladder. In still other embodiments, the sudden involuntary muscle contraction of the kidney. In yet other embodiments, the sudden involuntary muscle contraction of the bile duct. In still further embodiments, the spasmodic condition is a urinary tract spasm, gallstones, a gastrointestinal disorder, inflammatory bowel disorder (IBD), irritable bowel syndrome (IBS), renal colicky pain, or a spastic condition of the biliary tract. In other embodiments, the spasmodic condition is a urinary tract spasm. In further embodiments, the spasmodic condition is a gastrointestinal disorder. In still other embodiments, the spasmodic condition is an IBD. In yet further embodiments, the spasmodic condition is IBS. In other embodiments, the spasmodic condition is IBS with diarrhea (IBS-D). In further embodiments, the spasmodic condition is renal colicky pain. In yet other embodiments, the spasmodic condition is a spastic condition of the biliary tract. In still further embodiments, the spasmodic condition is mixed IBS (IBS-M). In other embodiments, the spasmodic condition is IBS with predominant constipation (IBS-C). In further embodiments, the spasmodic condition is Crohn's disease. In still other embodiments, the spasmodic condition is ulcerative colitis.

The pharmaceutical composition or oral dosage units may be administered to the human subject in a fed or fasted state. In some embodiments, the pharmaceutical composition or oral dosage unit is administered to the subject in a fed state. In other embodiments, the pharmaceutical composition or oral dosage unit is administered to the subject in a fasted state.

In certain aspects, oral administration of the pharmaceutical composition or oral dosage unit to the subject in a fed state results in a median Tₘₐₓ of the API of about 0.5 to about 1.75 hours; a mean Cₘₐₓ of the API of about 269 to about 1512 ng/ml; a mean AUCₜₐᵤ of the API of about 879 to about 4695 h*ng/mL; and/or a mean t½ of the API of about 1.6 hours to about 2.4 hours.

In some embodiments, the median Tₘₐₓ of the API in a fed state is about 0.5, about 0.75, about 1, about 1.25, about 1.5 or about 1.75 hours. In further embodiments, the median Tₘₐₓ of the API in a fed state is about 0.5 to about 1.5, about 0.5 to about 1, about 0.5 to about 0.75, about 0.75 to about 1.75, about 0.75 to about 1.5, about 0.75 to about 1.25, about 0.75 to about 1, about 1 to about 1.75, about 1 to about 1.5, about 1 to about 1.25, about 1.25 to about 1.75, about 1.25 to about 1.5, or about 1.5 to about 1.75 hours.

In other embodiments, the mean Cₘₐₓ of the API in a fed state is about 269, about 300, about 400, about 500, about 600, about 700, about 800, about 900, about 1000, about 1100, about 1200, about 1300, about 1400, about 1500, about 1512, or about 1550 ng/mL. In yet further embodiments, the Cₘₐₓ of the API in a fed state is about 269 to about 1550, about 269 to about 1512, about 269 to about 1500, about 269 to about 1400, about 269 to about 1300, about 269 to about 1200, about 269 to about 1100, about 269 to about 1000, about 269 to about 900, about 269 to about 800, about 269 to about 700, about 269 to about 600, about 269 to about 500, about 269 to about 400, about 300 to about 1550, about 300 to about 1500, about 300 to about 1512, about 300 to about 1500, about 300 to about 1400, about 300 to about 1400, about 300 to about 1200, about 300 to about 1100, about 300 to about 1000, about 300 to about 900, about 300 to about 800, about 300 to about 700, about 300 to about 600, about 300 to about 500, about 300 to about 400, about 400 to about 1550, about 400 to about 1512, about 400 to about 1500, about 400 to about 1400, about 400 to about 1300, about 400 to about 1200, about 400 to about 1100, about 400 to about 1000, about 400 to about 900, about 400 to about 800, about 400 to about 700, about 400 to about 600, about 400 to about 500, about 500 to about 1550, about 500 to about 1512, about 500 to about 1500, about 500 to about 1400, about 500 to about 1300, about 500 to about 1200, about 500 to about 1100, about 500 to about 1000, about 500 to about 900, about 500 to about 800, about 500 to about 700, about 500 to about 600, about 600 to about 1550, about 600 to about 1512, about 600 to about 1500, about 600 to about 1400, about 600 to about 1300, about 600 to about 1200, about 600 to about 1100, about 600 to about 1000, about 600 to about 900, about 600 to about 800, about 600 to about 700, about 700 to about 1550, about 700 to about 1512, about 700 to about 1500, about 700 to about 1400, about 700 to about 1300, about 700 to about 1200, about 700 to about 1100, about 700 to about 1000, about 700 to about 900, about 700 to about 800, about 800 to about 1550, about 800 to about 1512, about 800 to about 1500, about 800 to about 1400, about 800 to about 1300, about 800 to about 1200, about 800 to about 1100, about 800 to about 1000, about 800 to about 900, about 900 to about 1550, about 900 to about 1512, about 900 to about 1500, about 900 to about 1400, about 900 to about 1300, about 900 to about 1200, about 900 to about 1100, about 900 to about 1000, about 1000 to about 1550, about 1000 to about 1512, about 1000 to about 1500, about 1000 to about 1400, about 1000 to about 1300, about 1000 to about 1200, about 1000 to about 1100, about 1100 to about 1550, about 1100 to about 1512, about 1100 to about 1500, about 1100 to about 1400, about 1100 to about 1300, about 1100 to about 1200, about 1200 to about 1550, about 1200 to about 1500, about 1200 to about 1500, about 1200 to about 1400, about 1200 to about 1300, about 1300 to about 1550, about 1300 to about 1512, about 1300 to about 1500, about 1300 to about 1400, about 1400 to about 1550, about 1400 to about 1512, about 1400 to about 1500, or about 1500 to about 1550 ng/mL.

In other embodiments, the mean AUCₜₐᵤ of the API in a fed state is about 879 to about 4695 h*ng/ml. In further embodiments, the mean AUCₜₐᵤ of the API in a fed state is about 879, about 1000, about 1500, about 2000, about 2500, about 3000, about 3500, about 4000, about 4500, or about 4695 h*ng/mL. In yet other embodiments, the mean AUCₜₐᵤ of the API in a fed state is about 879 to about 4500, about 879 to about 4000, about 879 to about 3500, about 879 to about 3000, about 879 to about 2500, about 879 to about 2000, about 879 to about 1500, about 879 to about 1000, about 1000 to about 4695, about 1000 to about 4500, about 1000 to about 4000, about 1000 to about 3500, about 1000 to about 3000, about 1000 to about 2500, about 1000 to about 2000, about 1000 to about 1500, about 1500 to about 4695, about 1500 to about 4500, about 1500 to about 4000, about 1500 to about 3500, about 1500 to about 3000, about 1500 to about 2500, about 1500 to about 2000, about 2000 to about 4695, about 2000 to about 4500, about 2000 to about 4000, about 2000 to about 3500, about 2000 to about 3000, about 2000 to about 2500, about 2500 to about 4500, about 2500 to about 4695, about 2500 to about 4000, about 2500 to about 3500, about 2500 to about 3000, about 3000 to about 4695, about 3000 to about 4500, about 3000 to about 4000, about 3000 to about 3500, about 3500 to about 4695, about 3500 to about 4500, about 3500 to about 4000, about 4000 to about 4500, or about 4000 to about 4695 h*ng/mL.

In further embodiments, the mean t½ of the API in a fed state is about 1.6 hours to about 2.4 hours. In other embodiments, the mean t½ of the API in a fed state is about 1.6, about 1.7, about 1.8, about 1.9, about 2, about 2.1, about 2.2, about 2.3, or 2.4 hours. In yet further embodiments, the mean t½ of the API in a fed state is about 1.7 to about 2.3, about 1.6 to about 2.2, about 1.6 to about 2.1, about 1.6 to about 2, about 1.6 to about 1.9, about 1.6 to about 1.8, about 1.6 to about 1.7, about 1.7 to about 2.4, about 1.7 to about 2.3, about 1.7 to about 2.2, about 1.7 to about 2.1, about 1.7 to about 2, about 1.7 to about 1.9, about 1.7 to about 1.8, about 1.8 to about 2.4, about 1.8 to about 2.3, about 1.8 to about 2.2, about 1.8 to about 2.1, about 1.8 to about 2, about 1.8 to about 1.9, about 1.9 to about 2.4, about 1.9 to about 2.3, about 1.9 to about 2.2, about 1.9 to about 2.1, about 1.9 to about 2, about 2 to about 2.4, about 2 to about 2.3, about 2 to about 2.2, about 2 to about 2.1, about 2.1 to about 2.4, about 2.1 to about 2.3, about 2.1 to about 2.2, about 2.2 to about 2.4, about 2.2 to about 2.3, or about 2.3 to about 2.4 hours.

In other aspects, oral administration to the human subject in a fasted state results in a median Tₘₐₓ of the API of about 0.5 hours; a mean Cₘₐₓ of the API of about 2745 to about 4874 ng/ml; a mean AUCₜₐᵤ of the API of about 4567 to about 6853 h*ng/ml; and/or a mean t½ of the API of about 2 hours.

In some embodiments, oral administration to the human subject in a fasted state results in a median Tₘₐₓ of the API of about 0.5 hours.

In other embodiments, oral administration to the human subject in a fasted state results in a mean Cₘₐₓ of the API of about 2745 to about 4874 ng/ml. In further embodiments, the mean Cₘₐₓ of the API in a fasted state is about 2745, about 3000, about 3500, about 4000, about 4500, or about 4874 ng/mL. In still other embodiments, the mean Cₘₐₓ of the API in a fasted state is about 2745 to about 4500, about 2745 to about 4000, about 2745 to about 3500, about 2745 to about 3000, about 3000 to about 4874, about 3000 to about 4500, about 3000 to about 4000, about 3000 to about 3500, about 3500 to about 4874, about 3500 to about 4500, about 3500 to about 4000, about 4000 to about 4874, about 4000 to about 4500, or about 4500 to about 4874 ng/mL.

In yet further embodiments, oral administration to the subject in a fasted state results in a mean AUCₜₐᵤ of the API of about 4567 to about 6853 h*ng/ml. In other embodiments, the mean AUCₜₐᵤ of the API is about 4567, about 5000, about 5500, about 6000, about 6500, or about 6853 h*ng/mL. In further embodiments, the mean AUCₜₐᵤ of the API is about 4567 to about 6500, about 4567 to about 6000, about 4567 to about 5500, about 4567 to about 5000, about 5000 to about 6853, about 5000 to about 6500, about 5000 to about 6000, about 5000 to about 5500, about 5500 to about 6853, about 5500 to about 6500, about 5500 to about 6000, about 6000 to about 6853, about 6000 to about 6500, or about 6500 to about 6853 h*ng/mL.

In yet further embodiments, the mean t½ of the API in a fasted state is about 2 hours. In some embodiments, the mean t½ of the API in a fasted state is about 2, about 2.1, or about 2.2 hours. In other embodiments, the mean t½ of the API in a fasted state is about 2 to about 2.2, about 2 to about 2.1, or about 2.1 to about 2.2 hours.

| Abbre- | |
|---|---|
| viation | Definition |
| λz | Apparent first-order terminal elimination rate constant |
| Ae | Cumulative amount of phloroglucinol excreted in the urine |
| Ae_{0-10 h} | Amount excreted unchanged in urine from hour 0 to hour 10 |
| AE | Adverse event |
| AM | Morning |
| ATC | Anatomical Therapeutic Chemical |
| AUC | Area under the plasma concentration-time curve |
| AUC_{0-inf} | Area under the plasma concentration-time curve from time 0 |
| | to infinity |
| AUC₀₋ₗₐₛₜ | Area under the plasma concentration-time curve from time 0 |
| | to the time of the last quantifiable plasma concentration |
| AUC₀₋ₜ | Area under the plasma concentration-time curve from time 0 |
| | to time t |
| AUCₑₓₜᵣₐₚ | Percent of area under the plasma concentration-time curve |
| | extrapolated |
| AUCₜₐᵤ | Area under the plasma concentration-time curve over a dosing |
| | interval |
| BID | Two times per day |
| BLQ | Below the limit of quantification |
| BMI | Body mass index |
| bpm | Beats per minute |
| CI | Confidence interval |
| Cₗₐₛₜ | Last quantifiable plasma concentration |
| CL/F | Apparent plasma clearance |
| CLr | Clearance |
| Cₘₐₓ | Maximum plasma concentration |
| Cₘᵢₙ | Minimum plasma concentration |
| COVID- | Coronavirus Disease 2019 |
| 19 | |
| CTCAE | Common Terminology Criteria for Adverse Events |
| CV | Coefficient of variation |
| DR | Delayed release |
| ECG | Electrocardiogram |
| ET | Early Termination |
| Fe | Fraction of dose excreted |
| FSH | Follicle-stimulating hormone |
| Geo | Geometric |
| GM | Geometric mean |
| h | Hour |
| HR | Heart rate |
| IBS | Irritable bowel syndrome |
| IBS-D | Irritable bowel syndrome with diarrhea |
| IR | Immediate release |
| ln | Natural logarithm |
| LLOQ | lower limit of quantification |
| max | Maximum |
| MedDRA | Medical Dictionary for Regulatory Activities |
| NSAID | Nonsteroidal anti-inflammatory drug |
| OTC | Over-the-counter |
| PK | Pharmacokinetic(s) |
| PM | Evening |
| PT | Preferred term |
| QT | Heart rate corrected interval |
| QTcF | QT interval corrected using Fridericia's formula |
| REML | Restricted maximum likelihood. |
| RNA | Ribonucleic acid |
| SAE | Serious adverse event |
| SARS- | Severe acute respiratory syndrome coronavirus 2 |
| CoV-2 | |
| SD | Standard deviation |
| SOC | System organ class |
| t½ | Terminal phase elimination half-life |
| TEAE | Treatment-emergent adverse event |
| Tₘₐₓ | Time to maximum plasma concentration |
| Vd/F | Apparent volume of distribution |
| WHO | World Health Organization |

### Example 1: Modified-Release Formulation

The dosage form of the present disclosure contains three types of beads. One type of bead releases the phloroglucinol about immediately after dosing, a second type of bead release the phloroglucinol about 2 to 4 hours after dosing, and the third type of bead releases the phloroglucinol about 4 to about 6 hours after dosing. The amount and quantity of each component is found in Tables 1A and 1B.

TABLE 1A

| | | |
|---|---|---|
| | Quantity | Quantity |
| Component | mg/capsule | % w/w |

Core Bead Preparation

| | | |
|---|---|---|
| Phloroglucinol, anhydrous | 320 | 56.2 |
| Microcrystalline Cellulose, PH 101 | 123.0 | 21.6 |
| Hypromellose 2910-5 | 24.6 | 4.3 |
| Croscarmellose Sodium | 24.6 | 4.3 |
| Purified water¹ | Q.S | |

2 hour Release Coating Preparation

| | | |
|---|---|---|
| Eudragit L30D-55² | 28.4 | 5.0 |
| Triethyl Citrate² | 2.83 | 0.5 |
| Talc² USP/EP | 5.67 | 1.0 |
| Purified water² | | |

4 hour Release Coating Preparation

| | | |
|---|---|---|
| Eudragit FS 30D³ | 28.4 | 5.0 |
| Triethyl Citrate³ | 2.83 | 0.5 |
| Talc³ USP/EP | 5.68 | 1.0 |
| Purified water³ | | |

Lubrication

| | | |
|---|---|---|
| Talc, USP/EP | 3.0 | 0.5 |

Encapsulation (Opaque white size 00 capsules)

| | | |
|---|---|---|
| Total | 569.00 | 100.1 |

¹Removed during processing.
²Prepared as 30% dispersion containing 20% solids for coating weight gain of 30%. Solution prepared in excess.
³Prepared as 30% dispersion containing 20% solids for coating weight gain of 30%. Solution prepared in excess.

TABLE 1B

| | | | |
|---|---|---|---|
| | | Quantity | Quantity |
| | Quantity | % w/w in | % w/w |
| Component | mg/capsule | Capsule | in Bead |

Core Bead Preparation

| | | | |
|---|---|---|---|
| Phloroglucinol, anhydrous | 160 | 28.13 | 65 |
| Microcrystalline Cellulose, PH 101 | 61.5 | 10.81 | 25 |
| Hypromellose 2910-5 | 12.3 | 2.16 | 5 |
| Croscarmellose Sodium | 12.3 | 2.16 | 5 |
| Purified water¹ | Q.S | | |
| TOTAL in bead | 246.1 | — | 100 |

2 hour Release Bead

| | | | |
|---|---|---|---|
| Phloroglucinol, anhydrous | 80 | 14.06 | 50.01 |
| Microcrystalline Cellulose, PH 101 | 30.75 | 5.41 | 19.22 |
| Hypromellose 2910-5 | 6.15 | 1.08 | 3.84 |
| Croscarmellose Sodium | 6.15 | 1.08 | 3.84 |
| Eudragit L30D-55² | 28.4 | 4.99 | 17.75 |
| Triethyl Citrate² | 2.83 | 0.5 | 1.77 |
| Talc² USP/EP | 5.67 | 1 | 3.54 |
| Purified water² | | | |
| TOTAL in bead | 159.95 | 28.12 | 28.12 |

4 hour Release Bead

| | | | |
|---|---|---|---|
| Phloroglucinol, anhydrous | 80 | 14.06 | 50.01 |
| Microcrystalline Cellulose, PH 101 | 30.75 | 5.41 | 19.22 |
| Hypromellose 2910-5 | 6.15 | 1.08 | 3.84 |
| Croscarmellose Sodium | 6.15 | 1.08 | 3.84 |
| Eudragit FS 30D³ | 28.4 | 4.99 | 17.75 |
| Triethyl Citrate³ | 2.83 | 0.5 | 1.77 |
| Talc³ USP/EP | 5.68 | 1 | 3.54 |
| Purified water³ | | | |
| TOTAL in bead | 159.95 | — | 28.12 |

Lubrication

| | | | |
|---|---|---|---|
| Talc, USP/EP | 3.0 | 0.5 | 100 |

Encapsulation (Opaque white size 00 capsules)

| | |
|---|---|
| Total | 568.8 |

¹Removed during processing.
²Prepared as 30% dispersion containing 20% solids for coating weight gain of 30%. Solution prepared in excess.
³Prepared as 30% dispersion containing 20% solids for coating weight gain of 30%. Solution prepared in excess

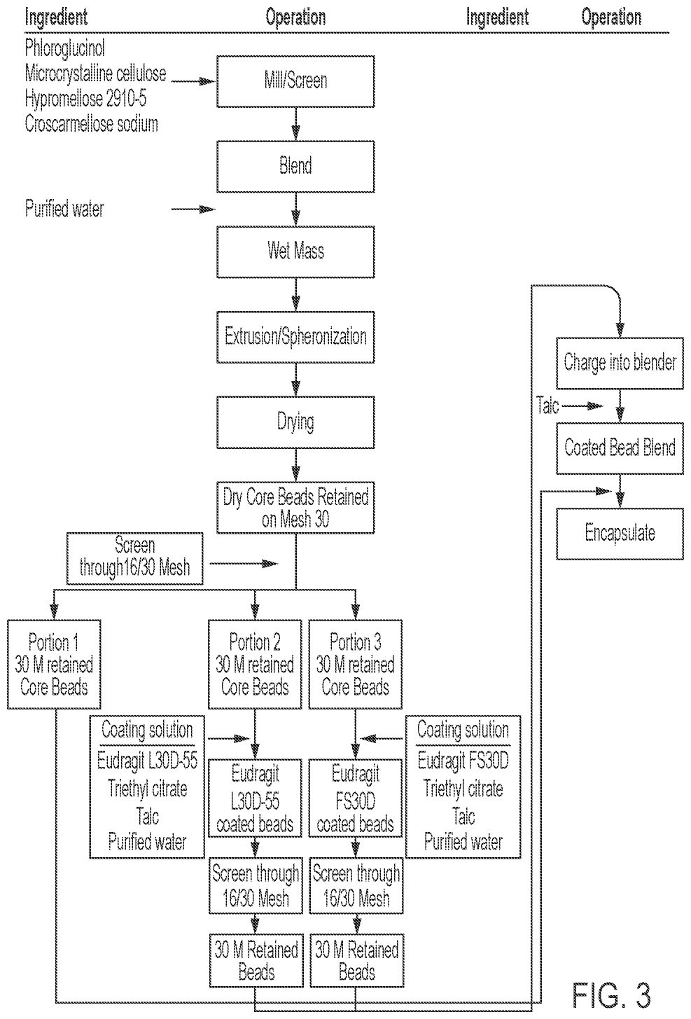

The beads were prepared as described in the flow diagram in FIG. 3.
A. The core beads, i.e., immediate release beads are prepared as follows:
1. Phloroglucinol, microcrystalline cellulose, hypromellose, and croscarmellose sodium are screened through an appropriate mill.
2. The milled mixture from step 1 is blended in a suitable granulator/mixer and a wet mass is prepared using purified water.
3. The wet mass is passed through an extruder/spheronizer to prepare wet beads.
4. The wet beads are dried using a suitable dryer to obtain core beads.
5. All of the core beads are screened through a 16/30 mesh and the beads that are retained on 30 mesh are collected as uncoated beads.
6. The beads from step 5 are divided into 3 Portions: Portion 1, Portion 2, and Portion 3.
B. Preparation of Eudragit L30D-55 Beads
7. A coating solution is prepared using Eudragit L30D-55, triethyl citrate, talc, and purified water.
8. The Portion 2 core beads from step 6 are coated using coating solution from step 7 to prepare Eudragit L30D-55 coated beads.
9. All beads from step 8 are screened through a 16/30 mesh and the beads retained on the 30 mesh are collected as Eudragit L30D-55 coated beads.
C. Preparation of Eudragit FS30D Beads
10. A coating solution is prepared using Eudragit FS30D, triethyl citrate, talc, and purified water.
11. The Portion 3 core beads from step 6 are coated using coating solution from step 10 to prepare Eudragit FS30D coated beads.
12. All beads from step 11 are screened through a 16/30 mesh and the beads retained on the 30 mesh are collected as Eudragit FS30D coated beads.
D. Lubrication
13. Eudragit L30D-55 coated beads from step 9 and Eudragit FS30D coated beads from step 12 are combined with talc into a suitable blender.
E. Encapsulation
14. The Portion 1 uncoated beads from step 6 and blend from step 13 are encapsulated into size 00 opaque white gelatin capsules using appropriate capsule filling machine.
15. The filled capsules are transferred through deduster and metal detector.

As previously described, the IR beads were coated with a suspension containing either Eudragit L30D55 producing DR beads intended to release at pH 5.5 or Eudragit FS30D-55 producing DR beads intended to release pH 7.0.

As previously discussed, the coated beads were blended and lubricated with talc and were encapsulated along with the IR beads into a size 00 Capsules. The Eudragit L30D-55 and Eudragit FS30D coated beads were blended together prior to encapsulation.

### Example 2

The immediate release beads, 2-hour modified release beads, and 4-hour modified release beads are prepared as described in Example 1. The placebo batch coated sugar spheres with Eudragit L30D-55 and Eudragit FS30D were prepared using sugar spheres as the core beads and coated as described in Example 1 for the phloroglucinol core beads. See, Table 2 for the components of the placebo capsules.

TABLE 2
Placebo Capsules

| | | |
|---|---|---|
| | Quantity | Quantity |
| Component | mg/capsule | % w/w |

Core Beads

| | | |
|---|---|---|
| Sugar Spheres 16-18 Mesh (PFO13) | 246.0 | 43.2 |

2 hour Release Beads

| | | |
|---|---|---|
| Sugar Spheres 16-18 Mesh (PFO13) | 123.0 | 21.6 |
| Eudragit L30D-55² | 28.4 | 5.0 |
| Triethyl Citrate² | 2.83 | 0.5 |
| Talc² USP/EP | 5.67 | 1.0 |
| Purified water² | | |

4 hour Release Beads

| | | |
|---|---|---|
| Sugar Spheres 16-18 Mesh (PFO13) | 123.0 | 21.6 |
| Eudragit FS 30D³ | 28.4 | 5.0 |
| Triethyl Citrate³ | 2.83 | 0.5 |
| Talc³ USP/EP | 5.68 | 1.0 |
| Purified water³ | | |

Lubrication

| | | |
|---|---|---|
| Talc, USP/EP | 3.0 | 0.5 |

Encapsulation (Opaque white size 00 capsules)
²Prepared as 30% dispersion containing 20% solids for coating weight gain of 30%. Solution prepared in excess.
³Prepared as 30% dispersion containing 20% solids for coating weight gain of 30%. Solution prepared in excess

The IR Beads, Eudragit L30D-55 coated beads, and Eudragit FS30D coated beads were assayed prior to blending and encapsulation steps.

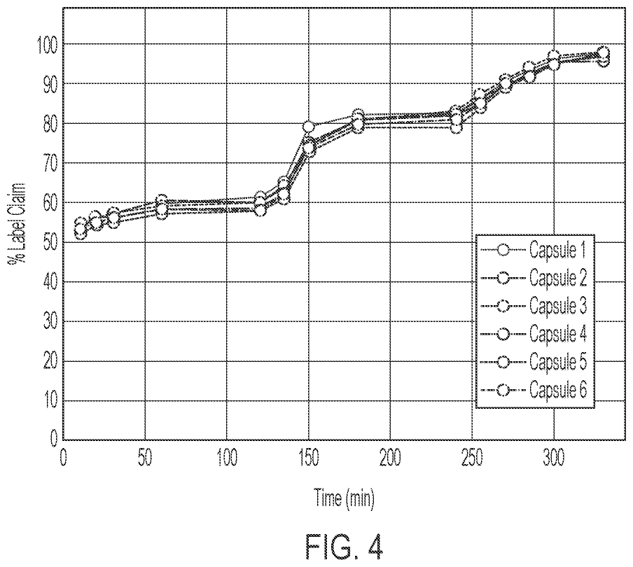

The finished capsules were tested using validated methods. The acceptance criteria for the active batches are shown in Table 3. All batches met acceptance criteria. The dissolution profiles for the individual, filled capsules is shown FIG. 4.

TABLE 3

| | Patheon | |
|---|---|---|
| Test | Method | Acceptance Criteria |
| Description | P14100 | Off-white size 00 capsule with |
| | | no markings |
| Assay by HPLC | 931485 | 90.0 to 110.0 |
| Related Substances | 931486 | Unknown ≤0.2% |
| | | Known ≤0.2% |
| | | Total Impurities ≤5.0% |
| Water Content by | 931488 | Report |
| Karl Fisher | | |
| Content Uniformity | 931485 | Meets USP <905> Requirement |
| Dissolution with | 931487 | NLT 30% (Q) of the labeled amount of |
| Quantitation by | | phloroglucinol dissolved at 120 minutes |
| HPLC | | NLT 60% (Q) of the labeled amount of |
| | | phloroglucinol dissolved at 240 minutes |
| | | NLT 30% (Q) of the labeled amount of |
| | | phloroglucinol dissolved at 30 minutes |
| Micro Testing | | |
| TAMC | P931076 | NMT 1000 |
| TYMC | | NMT 100 |
| E. coli | | Absent |

### Example 3

This was a randomized, double-blind, placebo-controlled, multiple ascending dose study to characterize the safety and PK of Formulation A when administered to healthy adult subjects. Formulation A was prepared as described in Example 1. A preliminary food effect assessment was also performed.

### A. Objectives

The objectives of this study were the following:
To assess the safety and tolerability of Formulation A following administration of multiple oral doses to healthy subjects; and
To characterize the PK of Formulation A following administration of multiple oral doses to healthy subjects.

Subjects were randomized to 1 of 8 cohorts with 6 subjects in each cohort scheduled to receive Formulation A and 2 subjects in each cohort receiving placebo as described below.
Cohort 1a: 320 mg Formulation A or matching placebo with a high fat breakfast (0 hour) each day and with a high fat dinner (10 hours) each day starting on the morning of Day 1 and continuing through the morning of Day 6;
Cohort 1b: 320 mg Formulation A or matching placebo with a low fat breakfast (0 hour) each day and with a low fat dinner (10 hours) each day starting on the morning of Day 1 and continuing through the morning of Day 6;
Cohort 2a: 640 mg Formulation A or matching placebo with a high fat breakfast (0 hour) each day and with a high fat dinner (10 hours) each day starting on the morning of Day 1 and continuing through the morning of Day 6;
Cohort 2b: 640 mg Formulation A or matching placebo with a low fat breakfast (0 hour) each day and with a low fat dinner (10 hours) each day starting on the morning of Day 1 and continuing through the morning of Day 6;
Cohort 3a: 1280 mg Formulation A or matching placebo with a high fat breakfast (0 hour) each day and with a high fat dinner (10 hours) each day starting on the morning of Day 1 and continuing through the morning of Day 6;
Cohort 3b: 1280 mg Formulation A or matching placebo with a low fat breakfast (0 hour) each day and with a low fat dinner (10 hours) each day starting on the morning of Day 1 and continuing through the morning of Day 6;
Cohort 3c: 960 mg Formulation A or matching placebo two times per day (BID) (at 0 hour and 10 hours) on an empty stomach each day starting on the morning of Day 1 and continuing through the morning of Day 6; and
Cohort 3d: 1280 mg Formulation A or matching placebo BID (at 0 hour and 10 hours) on an empty stomach each day starting on the morning of Day 1 and continuing through the morning of Day 6.

Meal definitions were in accordance with Food and Drug Administration Guidance on Assessing the Effects of Food on Drugs in Investigational New Drug Applications and New Drug Applications. See, Table 4.

TABLE 4
Meal Definitions

| | | Fat | | |
|---|---|---|---|---|
| Meal Type | Total Kcal | Kcal | Grams | Percent |
| High-Fat | 800-1000 | 500-600 | 55-65 | 50 |
| Low-Fat | 400-500 | 100-125 | 11-14 | 35 |

For each subject, the study consisted of the following:
A screening period of up to 26 days prior to the start of confinement for the inpatient period (Day-1);
A single 7-day inpatient treatment period, consisting of administration of blinded study drug (Formulation A or matching placebo capsules) from the morning of Day 1 through the morning of Day 6 followed by 24 hours of PK sampling; and
A follow-up telephone call 3 days (+1 day) after discharge from the clinical unit.

For each cohort, a sentinel dose group consisting of the first 2 subjects (1 subject received Formulation A and 1 subject received placebo) began the dosing regimen first. After a minimum of 48 hours, the remainder of the cohort began the dosing regimen. These remaining subjects in each cohort may have commenced dosing on the same day or may have been split into smaller groups which commenced dosing on multiple days.

For Cohorts 3c and 3d, where subjects received Formulation A or placebo on an empty stomach, the following order was observed:
Sentinel subjects in Cohort 3c (3 capsules on an empty stomach) commenced dosing at least 48 hours after the final subject in Cohort 3b had commenced dosing; and
Sentinel subjects in Cohort 3d (4 capsules on an empty stomach) commenced dosing at least 48 hours after the final subject in Cohort 3c had commenced dosing.

Progression from Cohort 1 to Cohort 2 and from Cohort 2 to Cohort 3 only took place after it was determined that adequate safety and tolerability had been demonstrated to permit proceeding.

Safety was assessed throughout the study based on AEs, physical examinations, skin and oral mucosa evaluations, ECGs, vital sign assessments, and clinical laboratory evaluations. Starting with Cohort 2, the number of daily bowel movements was recorded. Unscheduled procedures or visits and/or additional follow-up may have been required for subjects with clinically significant abnormal laboratory findings, unresolved TEAEs, SAEs that required follow-up laboratories and review, or clinically significant AEs.

### B. Inclusion Criteria

Subjects who met all the following criteria based on screening and check-in results were eligible to participate in the study:
1. Healthy subjects between the ages of 18 and 55 years, inclusive, in good health based on medical and psychiatric history, physical examination, ECG, vital signs, and routine laboratory tests (blood chemistry, hematology, coagulation, and urinalysis);
2. Body mass index (BMI) between 18 and 30 kg/m², inclusive;
3. Nonsmokers who had not used nicotine-containing products for at least 6 months prior to screening;
4. Male subjects with female partners of child-bearing potential must have agreed to use 2 medically accepted, highly effective methods of birth control from Day 1 through 90 days after the final dose of study drug. Medically accepted, highly effective methods of birth control for male subjects with female partners of child-bearing potential included the following: latex condom with spermicide, diaphragm with intravaginal spermicide, cervical cap with spermicide, indwelling intrauterine device (hormonal or nonhormonal), implanted contraceptives, and oral contraceptives;
5. Male subjects must have agreed to abstain from sperm donation from Day 1 through 90 days after administration of the final dose of study drug;
6. Female subjects with male partners must have been surgically sterile (hysterectomy and/or bilateral oophorectomy), postmenopausal for at least 1 year (with FSH in postmenopausal range), or agreed to use 2 medically accepted, highly effective methods of birth control from Day-14 until 60 days following the final dose of study drug. Medically accepted, highly effective methods of birth control for female subjects with male partners included the following: latex condom with spermicide, diaphragm with intravaginal spermicide, cervical cap with spermicide, and nonhormonal indwelling intrauterine device; and
7. Were able to understand and willing to comply with study procedures and restrictions (including confinement to the clinical unit, fasting and meal requirements, and restrictions on physical activity, use of recreational drugs or alcohol, and medications), and provided written informed consent according to institutional and regulatory guidelines.

### C. Exclusion Criteria

Subjects who met any of the following criteria based on screening and check-in results were excluded from participation in the study:
1. Were actively participating in an experimental therapy study; received experimental therapy with a small molecule within 30 days of the first dose of study drug or 5 half-lives, whichever was longer; or received experimental therapy with a large molecule within 90 days of the first dose of study drug or 5 half-lives, whichever was longer;
2. Had a personal or family history of long QT syndrome, Torsades de Pointes, or other complex ventricular arrhythmias, or family history of sudden death;
3. Had a history of or current clinically significant arrhythmias, including ventricular tachycardia, ventricular fibrillation, atrial fibrillation, sinus node dysfunction, or clinically significant heart block. Subjects with minor forms of ectopy (e.g., premature atrial contractions) were not necessarily excluded;
4. Had prolonged QTcF>450 msec based on the average of triplicate ECGs;
5. Had seated systolic blood pressure≥140 mmHg and/or diastolic blood pressure≥90 mmHg or systolic blood pressure<100 mmHg and/or diastolic blood pressure<50 mmHg;
6. Had a resting heart rate (HR) higher than 100 beats per minute (bpm) or lower than 60 bpm;
7. Had a temperature greater than 37.6° C. (99.7° F., measured orally), and respiration rate less than 12 or greater than 20 breaths/minute;
8. Were positive for human immunodeficiency virus antibody, hepatitis C virus antibody, hepatitis B surface antigen, or SARS-COV-2 RNA;
9. Had any other clinical laboratory values that were meaningfully outside of normal limits (based on laboratory normal range);
10. Had evidence or history of any clinically significant immunologic, hematologic, renal, endocrine, pulmonary, gastrointestinal, cardiovascular, musculoskeletal, hepatic, psychiatric, neurologic (including history of seizures or convulsions), or allergic disease (including clinically significant or multiple drug allergies); surgical conditions; cancer (with the exception of basal or squamous cell carcinoma of the skin and cancer that resolved or has been in remission for >5 years prior to screening); or any condition that may have confounded study procedures or results, impacted subject safety, or interfered with the absorption, distribution, metabolism, or excretion of the study drug (appendectomy allowed, cholecystectomy prohibited);
11. Had used any prescription medications (including topicals) or OTC medications (other than occasional use of acetaminophen or NSAIDs, such as ibuprofen or naproxen, according to the package insert); herbal supplements; dietary supplements; or nutraceuticals within 14 days prior to the first dose of study drug, or 5 half-lives, whichever was longer, or an unwillingness to refrain from these medications through final discharge from the clinical unit. Use of OTC topical medications may be permitted. In addition, medications for which 5 half-lives exceeded 14 days must have been approved prior to subject enrollment;
12. Had a positive drug or alcohol test result or a history of alcoholism or drug abuse within 2 years prior to the first dose of study drug as defined by the Diagnostic and Statistical Manual of Mental Disorders, 4th Edition: DSM-IV;
13. Had a typical consumption of ≥14 alcoholic drinks weekly. Note: 1 drink of alcohol was equivalent to ½ pint of beer (285 mL), 1 glass of spirits (25 mL), or 1 glass of wine (125 mL);
14. Had a history of or evidence of illicit drug use within the past 2 years;
15. Had surgical procedures within 4 weeks of check-in or planned elective surgery during the study period;
16. Had any illness during the 4 weeks before check-in unless deemed not clinically significant by the investigator;
17. Had a known allergy to any ingredient of Formulation A. Any history of severe allergic reaction (including drugs, food, insect bites, or environmental allergens);
18. Had inadequate venous access;
19. Was currently undergoing treatment with weight loss medication or prior weight loss surgery (e.g., gastric bypass surgery);
20. Was pregnant, breastfeeding, or planning to become pregnant during the study; or
21. Was considered to be unsuitable for any other reason that may have either placed the subject at increased risk during participation or interfere with the interpretation of the study outcomes.

### D. Treatments

Study subjects were dosed with either Formulation A or placebo capsules orally. The study subjects were randomized in a 6:2 ratio to receive Formulation A or placebo.

Each Formulation A capsule contained 320 mg of phloroglucinol in the following configuration: 160 mg in the IR beads, 80 mg in the 2-hour DR beads, and 80 mg in the 4-hour DR beads. The inactive ingredients used in the formulation included: microcrystalline cellulose, hypromellose, croscarmellose sodium, triethyl citrate, Eudragit L 30 D-55, and talc.

For a given dose cohort, placebo consisted of the same number of matching placebo capsules as the subjects receiving Formulation A. See, Table 5.

TABLE 5
Study Drug

| Study Drug | Dose and Mode of Administration |
|---|---|
| Formulation A | 320 mg capsules, orally |
| Placebo | 0 mg matching capsules, orally |

### (i) Selection and Timing of Dose for Each Subject

TABLE 6
Dose Cohorts

| | | | IR/DR/DR | | |
|---|---|---|---|---|---|
| | | | Doses of | | |
| | | | Phloroglucinol | Number of | |
| | Total per | Total per | (mg) in Each | Capsules | Meal |
| Cohort | Dose (mg) | Day (mg) | Capsule | per Dose | Conditions |
| 1a | 320 | 640 | 160/80/80 | 1 | High fat |
| 1b | 320 | 640 | 160/80/80 | 1 | Low fat |
| 2a | 620 | 1280 | 320/160/160 | 2 | High fat |
| 2b | 630 | 1280 | 320/160/160 | 2 | Low fat |
| 3a | 1280 | 2560 | 640/320/320 | 4 | High fat |
| 3b | 1280 | 2560 | 640/320/320 | 4 | Low fat |
| 3c | 960 | 1920 | 480/240/240 | 3 | Empty |
| | | | | | stomach |
| 3d | 1280 | 2560 | 640/320/320 | 4 | Empty |
| | | | | | stomach |

All subjects received multiple oral doses of blinded study drug (Formulation A or matching placebo capsules).

All subjects in all cohorts were required to observe an overnight fast of at least 10 hours starting on the evenings prior to the first and final doses of study drug.

For subjects in the fed cohorts, the relevant meal type was administered starting 30 minutes prior to the corresponding dose and the full meal must have been consumed prior to dosing. Subjects also received lunch each day a minimum of 4 hours after the morning study drug administration. No study drug was administered with lunch. Lunch contents were standard but not strictly restricted to the high fat or low fat criteria.

For cohorts required to take study drug on an empty stomach, no food may have been consumed for a minimum of 2 hours before each interim dose or 1 hour after each interim dose. Subjects received lunch each day a minimum of 4 hours after study drug administration. All meals were standardized.

Subjects must have consumed all study drug capsules in ≤5 minutes. Study drug was administered with approximately 240 mL of water.

All subjects received standardized meals scheduled at approximately the same time each day (when a protocol-specified fast was not in effect) during confinement. Subjects were encouraged to remain sedentary and required an escort if he/she needed to use the restroom during the first 3.5 hours postdose.

(ii) Excluded Medications and/or Procedures

Prescription medications (including topicals) or OTC medications (other than occasional use of acetaminophen or NSAIDs, such as ibuprofen or naproxen, according to the package insert); herbal supplements; dietary supplements; or nutraceuticals were prohibited within 14 days prior to the first dose of study drug, or 5 half-lives, whichever was longer, until discharge except for those required for treatment of AEs. Use of OTC topical medications may have been permitted. In addition, medications for which 5 half-lives exceeded 14 days may have been approved.

Subjects may not have undergone treatment with weight loss medication during the study or have had prior weight loss surgery (e.g., gastric bypass surgery).

Subjects could not participate in any other experimental therapy study while participating in this study; receive experimental therapy with a small molecule within 30 days of the first dose of study drug, or 5 half-lives, whichever was longer; or receive experimental therapy with a large molecule within 90 days of the first dose of study drug, or 5 half-lives, whichever was longer.

(iii) General and Dietary Restrictions

All subjects in all cohorts were required to observe an overnight fast of at least 10 hours starting on the evenings prior to the first and final doses of study drug.

All interim doses were administered on an empty stomach (at least approximately 1 hour before a meal or approximately 2 hours after a meal). Water was permitted during the fast except for 1 hour prior to dosing and 1 hour after dosing (with the exception of water used to administer study drug). Subjects must have consumed all study drug capsules in ≤5 minutes. Study drug was administered with approximately 240 mL of water.

Subjects received standardized meals scheduled at approximately the same time each day (when a protocol-specified fast was not in effect) during confinement. Subjects were encouraged to remain sedentary and required an escort if he/she needed to use the restroom during first 3.5 hours postdose.

Subjects must have abstained from alcohol; caffeine and/or xanthine-containing products (i.e., coffee, tea, chocolate, and caffeine-containing sodas, colas, energy drinks, etc.); grapefruit, grapefruit products, star fruit, star fruit products, and Seville oranges; and vitamin waters from 48 hours prior to the dose of study drug on Day 1 through final discharge from the clinical unit. Subjects must have refrained from contact sports and strenuous exercise beginning 5 days prior to the first dose of study drug and throughout the confinement period. Subjects must have been nonsmokers who had not used nicotine containing products for at least 6 months prior to screening.

(iii) Pharmacokinetic Endpoints

The following plasma PK parameters were determined for phloroglucinol following the first dose of Formulation A on the morning of Day 1, as the data permitted:
Cₘₐₓ;
Tₘₐₓ;
AUCₜₐᵤ; and
Cₘᵢₙ

The following plasma PK parameters were determined for phloroglucinol following the final dose of Formulation A on the morning of Day 6, as the data permitted:
Cₘₐₓ;
Tₘₐₓ;
AUCₜₐᵤ;
AUC_{0-inf} and associated percent % extrapolated;
AUC₀₋ₜ;
t½;
λz;
CL/F (based on AUCₜₐᵤ); and
Vd/F (based on AUCₜₐᵤ).

In addition, trough samples were used to assess attainment of steady state.

Accumulation at steady state was also characterized based on relevant Cₘₐₓ and AUC values.

The cumulative amount of phloroglucinol excreted in the urine (Ae), renal clearance (calculated as Ae/AUCₜₐᵤ), and fraction of dose excreted (Fe) renally were also calculated using urine concentrations of phloroglucinol, as the data permitted.

Exploratory analyses to characterize the plasma and urine PK profile of the sulfate and glucuronide conjugates of phloroglucinol may have also been undertaken.

### (iv). Safety Endpoints

The safety of Formulation A was assessed from the time of informed consent until study participation was complete. The safety endpoints included the following:
Physical examinations, skin and oral mucosa evaluations, ECGs, vital sign assessments (seated and supine separately), and clinical laboratory evaluations;
TEAEs graded according to CTCAE Version 5.0 for severity;
Treatment-emergent SAEs;
TEAEs leading to premature discontinuation from the study;
Treatment-emergent marked laboratory abnormalities; and
Treatment-emergent marked vital sign abnormalities.

### (v) Statistical and Analytical Plans

Categorical data were generally summarized with counts and percentages of subjects. The denominator used for the percentage calculation was clearly defined. Continuous data (quantitative safety data or the difference from baseline) were generally summarized with descriptive statistics including n (number of non-missing values), mean, median, standard deviation, minimum, and maximum. Geometric mean (GM) and GM % coefficient of variation (CV) were also provided for the summary of concentrations and PK parameters. The subjects with 0 value were excluded from the calculation of GM and GM CV %

Summaries by cohort included all 8 cohorts separately while summaries by dose level combined respective cohorts for total dose levels of 320 mg, 640 mg, 960 mg, and 1280 mg.

Analysis day was calculated from the date of first dose of study drug. The day of the first dose of study drug was Treatment Period Day 1, and the day immediately before Day 1 was Day-1. There was no Day 0.

Baseline was defined as the last measurement prior to the first dose of study drug.

### (a) Analysis Populations

The Safety Population consisted of all randomized subjects who received at least 1 dose of Formulation A.

The PK Population included all subjects who received Formulation A and had at least 1 quantifiable postdose plasma concentration for phloroglucinol.

The PK Evaluable Population included subjects who had sufficient plasma concentration data to characterize at least 1 PK parameter for phloroglucinol.

Counts and percentages of subjects in each analysis population were summarized by cohort, dose level, and in total based on all randomized subjects.

### (b) Subject Disposition

Subject disposition was presented for all randomized subjects. The number and percentage of subjects in each of the following categories were summarized by cohort and overall, as appropriate:
Randomized;
Dosed;
Completed the study;
Prematurely withdrew from the study and the primary reasons for early withdrawal; and
Prematurely withdrew from the study due to COVID-19 pandemic.

### (ii) Protocol Deviations

Protocol deviations were summarized with frequency distributions (counts and percentages) by cohort, dose level, and category for all randomized subjects.

Subjects with protocol deviations were listed by cohort for all randomized subjects.

Any effects of COVID-19 on study visits (e.g., not completed, in-person partially completed, performed virtually, performed out of window) were listed for all subjects in the Safety Population.

### (c) Demographic and Baseline Characteristics

The following demographic and baseline characteristics were listed and summarized by cohort, dose level, and overall with descriptive statistics or counts and percentages of subjects for the Safety Population and repeated for all other analysis populations if they were different from the Safety Population:
Age (years);
Sex;
Childbearing potential;
Race (Asian, American Indian or Alaska Native, Black or African American, Native Hawaiian or Other Pacific Islander, White, Other);
Ethnicity (Hispanic or Latino, Not Hispanic or Latino, Not reported, Unknown);
Height (cm);
Weight (kg); and
BMI (kg/m²).

### Medical History

### (d) Medical History was Coded to SOC and PT Using the MedDRA Version 23.1.

Counts and percentages of subjects with medical history by SOC and PT were summarized by cohort, dose level, and in total based on all randomized subjects as well as listed.

### (e) Prior and Concomitant Medications

Prior and concomitant medications were coded to ATC class and PT using the WHO Drug Dictionary version September 2020GB3. For summary purposes, medications were considered prior medications if they stopped prior to the dose of study drug and concomitant medications if they were taken at any time after the first dose of study drug (i.e., started prior to the first dose of study drug and were ongoing or started after the first dose of study drug).

Counts and percentages of subjects taking prior and concomitant medications by ATC class and PT were listed and summarized by cohort, dose level, and in total based on the Safety Population.

### (vi) Study Drug Exposure and Compliance

Exposure was defined as the number of days between the first dose and last dose of study drug (inclusive) and was summarized by treatment for the Safety Population. Study drug administration data were listed by treatment group for all subjects in the Safety Population.

### (f) Pharmacokinetic Analyses

### Sample Collections for Pharmacokinetic Analysis

The predose blood sample for PK was collected within 60 minutes prior to the first administration of study drug and within 5 minutes prior to any specified subsequent administrations of study drug. The following windows were permitted for the collection of PK samples: ±1 minute for samples collected≤8 hours postdose, +2 minutes for samples collected>8 and ≤16 hours postdose, and +5 minutes for samples collected>16 hours postdose. The planned blood sample collection times (in hours from dosing) were:
Day 1 (first dose): predose, 0.25, 0.5, 0.75, 1, 1.5, 2, 2.25, 2.5, 2.75, 3, 3.5, 4, 4.25, 4.5, 4.75, 5, 5.5, 6, 6.5, 7, 7.5, 8, and 9;
Day 1 (evening dose): predose, 0.25, 0.5, 0.75, 1, 1.5, 2, 2.5, 3, 3.5, 4, 4.5, 5, 5.5, and 6;
Day 2 (morning dose): predose

Days 4 and 5 (morning and evening doses): predose; and
Day 6: predose, 0.25, 0.5, 0.75, 1, 1.5, 2, 2.25, 2.5, 2.75, 3, 3.5, 4, 4.25, 4.5, 4.75, 5, 5.5, 6, 6.5, 7, 7.5, 8, 9, 10, 11, 12, 16, and 24.

Urine samples for PK were collected for measurement of phloroglucinol concentrations during the following intervals after the first and final doses of study drug: 0 to 2 hours, 2 to 4 hours, 4 to 6 hours, 6 to 10 hours, 10 to 12 hours, 12 to 14 hours, 14 to 16 hours, and 16 to 24 hours. A urine aliquot was also obtained from the morning void prior to the first and final doses of study drug.

### Pharmacokinetic Concentration

Individual plasma concentrations of phloroglucinol were summarized by cohort at each nominal time point for the PK Population descriptively. Individual plasma concentrations were also listed for the PK Population.

Individual plasma concentration was plotted by cohort on a linear and semi-logarithmic scale against actual sampling time points relative to dosing time. Lower limit of quantification was plotted as a reference line in both instances. Troughs on Days 4, 5, and 6 were plotted in a similar manner.

Plots of mean concentrations of plasma phloroglucinol versus time were generated for each cohort. Plots of mean and individual cumulative Ae over time were also presented. Similar outputs were provided for any measured metabolites, if the data permitted.

Actual sampling times that were outside the sampling time windows may have been excluded from concentration summary and mean concentration plotting but were still used in the calculations of PK parameters and individual concentration plotting. Exclusion of concentrations that were outside of window were evaluated on a case by case basis.

### Pharmacokinetic Parameters

The following plasma PK parameters were determined (using non-compartmental analysis) for phloroglucinol following the first dose of Formulation A on the morning of Day 1, as the data permitted:
Cₘₐₓ—determined directly from the concentration time profile; if the Cₘₐₓ occurred at more than one time point, Cₘₐₓ was defined as the first maximum value;
Tₘₐₓ—if the maximum value occurred at more than one time point, Tₘₐₓ was defined as the first time point with this value;
AUCₜₐᵤ; and
Cₘᵢₙ.

The following plasma PK parameters were determined (using non-compartmental analysis) for phloroglucinol following the final dose of Formulation A on the morning of Day 6, as the data permitted:
Cₘₐₓ—determined directly from the concentration time profile; if the Cₘₐₓ occurred at more than one time point, Cₘₐₓ was defined as the first maximum value;
Tₘₐₓ—if the maximum value occurred at more than one time point, Tₘₐₓ was defined as the first time point with this value;
AUCₜₐᵤ;
AUC_{0-inf}—calculated as ([AUC₀₋ₜ]+last quantifiable plasma

concentration [Cₗₐₛₜ]/λ_{z});
AUC₀₋ₜ—AUC from predose (time 0) to Cₗₐₛₜ;
λ_{z}—calculated from a semi-logarithmic plot of the plasma concentration versus time curve. The parameter was calculated by linear least squares regression analysis using points in the terminal logarithmic-linear phase;
t½-calculated as natural logarithm (2)/λ_{z};
CL/F—calculated as Dose/AUCₜₐᵤ;
Vd/F—calculated as Dose/[λ_{z}·AUCₜₐᵤ];
R_{Cmax}—accumulation ratios assessment on Day 6 only; calculated as Cₘₐₓ on Day 6/Cₘₐₓ on Day 1;
R_{AUCtau}—accumulation ratios assessment on Day 6 only; calculated as AUCₜₐᵤ on Day 6/AUCₜₐᵤ on Day 1; and
AUCₑₓₜᵣₐₚ—percent of area under the plasma concentration-time curve extrapolated; represented as (1−AUC₀₋ₜ/AUC_{0-inf})·100.

The following urine PK parameters of phloroglucinol were determined using non-compartmental methods as appropriate:
Amount excreted unchanged in urine from hour 0 to hour 2 (Ae₀₋₂);
Amount excreted unchanged in urine from hour 0 to hour 4 (Ae₀₋₄);
Amount excreted unchanged in urine from hour 0 to hour 6 (Ae₀₋₆);
Amount excreted unchanged in urine from hour 0 to hour 10 (Ae₀₋₁₀);
Amount excreted unchanged in urine from hour 0 to hour 12 (Ae₀₋₁₂);
Amount excreted unchanged in urine from hour 0 to hour 14 (Ae₀₋₁₄);
Amount excreted unchanged in urine from hour 0 to hour 16 (Ae₀₋₁₆);
Amount excreted unchanged in urine from hour 0 to hour 24 (Ae₀₋₂₄);
Fraction of dose excreted unchanged in urine from hour 0 to hour 2 (Fe₀₋₂);
Fraction of dose excreted unchanged in urine from hour 0 to hour 4 (Fe₀₋₄);
Fraction of dose excreted unchanged in urine from hour 0 to hour 6 (Fe₀₋₆);
Fraction of dose excreted unchanged in urine from hour 0 to hour 10 (Fe₀₋₁₀);
Fraction of dose excreted unchanged in urine from hour 0 to hour 12 (Fe₀₋₁₂);
Fraction of dose excreted unchanged in urine from hour 0 to hour 14 (Fe₀₋₁₄);
Fraction of dose excreted unchanged in urine from hour 0 to hour 16 (Fe₀₋₁₆);
Fraction of dose excreted unchanged in urine from hour 0 to hour 24 (Fe₀₋₂₄); and
· Renal clearance; calculated as Ae/AUCₜₐᵤ.

Actual collection times were used in PK parameter calculations.

### E. Study Subjects

### (i) Disposition of Subjects

Table 7 presents subject disposition for the Safety Population.

A total of 65 subjects were randomized to 1 of 8 cohorts. Six subjects in each cohort received Formulation A at one of the following dose levels with a high or low fat meal (fed state): 320 mg, 640 mg, or 1280 mg. Two subjects in each cohort received placebo in a corresponding fed state. Six subjects received 960 mg of Formulation A and 6 subjects received 1280 mg of Formulation A on an empty stomach; and 2 subjects in each of these cohorts received placebo on an empty stomach. One additional subject in Cohort 3c, who received 960 mg Formulation A BID on an empty stomach, withdrew early from the study due to concerns with being able to tolerate the number of blood draws during the treatment period. The remaining 64 subjects completed the study.

TABLE 7
Subject Disposition - Safety Population

| | High Fat | | | | Low Fat | | | |
|---|---|---|---|---|---|---|---|---|
| | | | | Pooled | | | | Pooled |
| | 320 mg | 640 mg | 1280 mg | Placebo | 320 mg | 640 mg | 1280 mg | Placebo |
| | (N = 6) | (N = 6) | (N = 6) | (N = 6) | (N = 6) | (N = 6) | (N = 6) | (N = 6) |
| Category | n (%) | n (%) | n (%) | n (%) | n (%) | n (%) | n (%) | n (%) |
| Randomized | 6 | 6 | 6 | 6 | 6 | 6 | 6 | 6 |
| | (100.0) | (100.0) | (100.0) | (100.0) | (100.0) | (100.0) | (100.0) | (100.0) |
| Dosed | 6 | 6 | 6 | 6 | 6 | 6 | 6 | 6 |
| | (100.0) | (100.0) | (100.0) | (100.0) | (100.0) | (100.0) | (100.0) | (100.0) |
| Completed | 6 | 6 | 6 | 6 | 6 | 6 | 6 | 6 |
| the study | (100.0) | (100.0) | (100.0) | (100.0) | (100.0) | (100.0) | (100.0) | (100.0) |
| Early | 0 | 0 | 0 | 0 | 0 | 0 | 0 | 0 |
| termination | (0.0) | (0.0) | (0.0) | (0.0) | (0.0) | (0.0) | (0.0) | (0.0) |
| of the study | | | | | | | | |

Reason for early termination of the study

| | | | | | | | | |
|---|---|---|---|---|---|---|---|---|
| Withdrawal | 0 | 0 | 0 | 0 | 0 | 0 | 0 | 0 |
| by subject | (0.0) | (0.0) | (0.0) | (0.0) | (0.0) | (0.0) | (0.0) | (0.0) |
| Primary | 0 | 0 | 0 | 0 | 0 | 0 | 0 | 0 |
| reason for | (0.0) | (0.0) | (0.0) | (0.0) | (0.0) | (0.0) | (0.0) | (0.0) |
| early | | | | | | | | |
| termination | | | | | | | | |
| was due to | | | | | | | | |
| COVID-19 | | | | | | | | |

TABLE 7
Subject Disposition - Safety Population (continued)

| | | | | Overall | | | | |
|---|---|---|---|---|---|---|---|---|
| | Fasted | | | | | | | Total |
| | | | Pooled | Total | Total | Total | Total | Pooled |
| | 960 mg | 1280 mg | Placebo | 320 mg | 640 mg | 960 mg | 1280 mg | Placebo |
| | (N = 7) | (N = 6) | (N = 4) | (N = 12) | (N = 12) | (N = 7) | (N = 18) | (N = 16) |
| Category | n (%) | n (%) | n (%) | n (%) | n (%) | n (%) | n (%) | n (%) |
| Randomized | 7 | 6 | 4 | 12 | 12 | 7 | 18 | 16 |
| | (100.0) | (100.0) | (100.0) | (100.0) | (100.0) | (100.0) | (100.0) | (100.0) |
| Dosed | 7 | 6 | 4 | 12 | 12 | 7 | 18 | 16 |
| | (100.0) | (100.0) | (100.0) | (100.0) | (100.0) | (100.0) | (100.0) | (100.0) |
| Completed the | 6 | 6 | 4 | 12 | 12 | 6 | 18 | 16 |
| study | (85.7) | (100.0) | (100.0) | (100.0) | (100.0) | (85.7) | (100.0) | (100.0) |
| Early termination | 1 | 0 | 0 | 0 | 0 | 1 | 0 | 0 |
| of the study | (14.3) | (0.0) | (0.0) | (0.0) | (0.0) | (14.3) | (0.0) | (0.0) |

Reason for early termination of the study

| | | | | | | | | |
|---|---|---|---|---|---|---|---|---|
| Withdrawal by | 1 | 0 | 0 | 0 | 0 | 1 | 0 | 0 |
| subject | (14.3) | (0.0) | (0.0) | (0.0) | (0.0) | (14.3) | (0.0) | (0.0) |
| Primary reason | 0 | 0 | 0 | 0 | 0 | 0 | 0 | 0 |
| for early | (0.0) | (0.0) | (0.0) | (0.0) | (0.0) | (0.0) | (0.0) | (0.0) |
| termination was | | | | | | | | |
| due to | | | | | | | | |
| COVID-19 | | | | | | | | |

The percentage (%) was calculated as 100 × n/N. Subject 001-246 replaced Subject 001-243 in Cohort 3c (960 mg fasted).

### (ii) Demographic and Other Baseline Characteristics

Table 8 summarizes the demographic and baseline characteristics for the Safety Population. The mean age of subjects ranged from 32.3 to 42.5 years, and the mean BMI ranged from 22.64 to 28.63 kg/m². The majority of subjects were not Hispanic or Latino. Demographic and baseline characteristics were generally well matched across treatment groups.

TABLE 8
Demographic and Baseline Characteristics - Safety Population

| | | | | | Low Fat | | | |
|---|---|---|---|---|---|---|---|---|
| | High Fat | | | | | | | Total |
| | | | | Pooled | | | | Pooled |
| Characteristic | 320 mg | 640 mg | 1280 mg | Placebo | 320 mg | 640 mg | 1280 mg | Placebo |
| Statistic/Category | (N = 6) | (N = 6) | (N = 6) | (N = 6) | (N = 6) | (N = 6) | (N = 6) | (N = 6) |

Age at informed consent (years)

| | | | | | | | | |
|---|---|---|---|---|---|---|---|---|
| n | 6 | 6 | 6 | 6 | 6 | 6 | 6 | 6 |
| Mean | 37.2 | 33.3 | 42.5 | 40.0 | 35.8 | 40.2 | 40.5 | 35.5 |
| SD | 7.76 | 11.11 | 5.36 | 11.30 | 13.20 | 10.57 | 12.88 | 6.47 |
| Median | 37.5 | 29.5 | 41.0 | 39.0 | 35.5 | 37.5 | 45.0 | 33.0 |
| Minimum | 28 | 23 | 37 | 27 | 20 | 28 | 22 | 30 |
| Maximum | 48 | 52 | 50 | 55 | 53 | 53 | 52 | 47 |

Race - n (%)

| | | | | | | | | |
|---|---|---|---|---|---|---|---|---|
| White | 2 | 5 | 3 | 3 | 3 | 1 | 3 | 3 |
| | (33.3) | (83.3) | (50.0) | (50.0) | (50.0) | (16.7) | (50.0) | (50.0) |
| Black or African | 3 | 1 | 2 | 2 | 3 | 5 | 3 | 3 |
| American | (50.0) | (16.7) | (33.3) | (33.3) | (50.0) | (83.3) | (50.0) | (50.0) |
| American Indian or | 0 | 0 | 1 | 1 | 0 | 0 | 0 | 0 |
| Alaskan native | (0.0) | (0.0) | (16.7) | (16.7) | (0.0) | (0.0) | (0.0) | (0.0) |
| Other | 1 | 0 | 0 | 0 | 0 | 0 | 0 | 0 |
| | (16.7) | (0.0) | (0.0) | (0.0) | (0.0) | (0.0) | (0.0) | (0.0) |

Ethnicity - n (%)

| | | | | | | | | |
|---|---|---|---|---|---|---|---|---|
| Hispanic or Latino | 0 | 1 | 1 | 1 | 0 | 0 | 2 | 1 |
| | (0.0) | (16.7) | (16.7) | (16.7) | (0.0) | (0.0) | (33.3) | (16.7) |
| Not Hispanic or | 6 | 5 | 5 | 5 | 6 | 6 | 4 | 5 |
| Latino | (100.0) | (83.3) | (83.3) | (83.3) | (100.0) | (100.0) | (66.7) | (83.3) |

Sex - n (%)

| | | | | | | | | |
|---|---|---|---|---|---|---|---|---|
| Female | 3 | 2 | 2 | 3 | 2 | 4 | 2 | 1 |
| | (50.0) | (33.3) | (33.3) | (50.0) | (33.3) | (66.7) | (33.3) | (16.7) |
| Male | 3 | 4 | 4 | 3 | 4 | 2 | 4 | 5 |
| | (50.0) | (66.7) | (66.7) | (50.0) | (66.7) | (33.3) | (66.7) | (83.3) |

Body weight (kg)

| | | | | | | | | |
|---|---|---|---|---|---|---|---|---|
| n | 6 | 6 | 6 | 6 | 6 | 6 | 6 | 6 |
| Mean | 68.50 | 79.03 | 68.63 | 80.43 | 67.48 | 82.00 | 74.63 | 76.65 |
| SD | 10.472 | 12.573 | 10.773 | 9.455 | 9.903 | 7.219 | 11.379 | 6.291 |
| Median | 74.00 | 80.85 | 70.70 | 82.50 | 66.35 | 81.00 | 71.40 | 77.45 |
| Minimum | 54.8 | 55.6 | 55.8 | 67.4 | 54.2 | 72.3 | 60.1 | 67.3 |
| Maximum | 77.5 | 90.5 | 79.4 | 93.1 | 82.8 | 93.0 | 91.3 | 85.6 |

BMI (kg/m²)

| | | | | | | | | |
|---|---|---|---|---|---|---|---|---|
| n | 6 | 6 | 6 | 6 | 6 | 6 | 6 | 6 |
| Mean | 24.57 | 26.54 | 24.34 | 28.63 | 22.64 | 27.04 | 26.41 | 24.90 |
| SD | 1.947 | 3.104 | 2.798 | 0.708 | 2.488 | 2.021 | 2.526 | 2.209 |
| Median | 25.23 | 26.87 | 24.78 | 28.64 | 22.27 | 27.57 | 25.35 | 24.24 |
| Minimum | 21.6 | 20.7 | 20.1 | 27.8 | 19.6 | 24.4 | 23.8 | 23.2 |
| Maximum | 26.7 | 29.5 | 27.5 | 29.4 | 26.6 | 29.0 | 29.8 | 29.1 |

TABLE 8
Demographic and Baseline Characteristics - Safety Population (continued)

| | Fasted | | | Overall | | | | |
|---|---|---|---|---|---|---|---|---|
| | | | Pooled | Total | Total | Total | Total | Total Pooled |
| Characteristic | 960 mg | 1280 mg | Placebo | 320 mg | 640 mg | 960 mg | 1280 mg | Placebo |
| Statistic/Category | (N = 7) | (N = 6) | (N = 4) | (N = 12) | (N = 12) | (N = 7) | (N = 18) | (N = 16) |

Age at informed consent (years)

| | | | | | | | | |
|---|---|---|---|---|---|---|---|---|
| n | 7 | 6 | 4 | 12 | 12 | 7 | 18 | 16 |
| Mean | 32.3 | 41.2 | 41.3 | 36.5 | 36.8 | 32.3 | 41.4 | 38.6 |
| SD | 11.04 | 8.30 | 5.91 | 10.34 | 10.94 | 11.04 | 8.85 | 8.37 |
| Median | 29.0 | 40.0 | 42.0 | 36.0 | 33.0 | 29.0 | 43.0 | 39.0 |
| Minimum | 21 | 32 | 34 | 20 | 23 | 21 | 22 | 27 |
| Maximum | 53 | 51 | 47 | 53 | 53 | 53 | 52 | 55 |

Race - n (%)

| | | | | | | | | |
|---|---|---|---|---|---|---|---|---|
| White | 5 | 3 | 1 | 5 | 6 | 5 | 9 | 7 |
| | (71.4) | (50.0) | (25.0) | (41.7) | (50.0) | (71.4) | (50.0) | (43.8) |
| Black or African | 2 | 2 | 3 | 6 | 6 | 2 | 7 | 8 |
| American | (28.6) | (33.3) | (75.0) | (50.0) | (50.0) | (28.6) | (38.9) | (50.0) |
| American Indian | 0 | 0 | 0 | 0 | 0 | 0 | 1 | 1 |
| or Alaska native | (0.0) | (0.0) | (0.0) | (0.0) | (0.0) | (0.0) | (5.6) | (6.3) |
| Asian | 0 | 1 | 0 | 0 | 0 | 0 | 1 | 0 |
| | (0.0) | (16.7) | (0.0) | (0.0) | (0.0) | (0.0) | (5.6) | (0.0) |
| Other | 0 | 0 | 0 | 1 | 0 | 0 | 0 | 0 |
| | (0.0) | (0.0) | (0.0) | (8.3) | (0.0) | (0.0) | (0.0) | (0.0) |

Ethnicity - n (%)

| | | | | | | | | |
|---|---|---|---|---|---|---|---|---|
| Hispanic or | 1 | 0 | 1 | 0 | 1 | 1 | 3 | 3 |
| Latino | (14.3) | (0.0) | (25.0) | (0.0) | (8.3) | (14.3) | (16.7) | (18.8) |
| Not Hispanic or | 6 | 6 | 3 | 12 | 11 | 6 | 15 | 13 |
| Latino | (85.7) | (100.0) | (75.0) | (100.0) | (91.7) | (85.7) | (83.3) | (81.3) |

Sex - n (%)

| | | | | | | | | |
|---|---|---|---|---|---|---|---|---|
| Female | 2 | 0 | 1 | 5 | 6 | 2 | 4 | 5 |
| | (28.6) | (0.0) | (25.0) | (41.7) | (50.0) | (28.6) | (22.2) | (31.3) |
| Male | 5 | 6 | 3 | 7 | 6 | 5 | 14 | 11 |
| | (71.4) | (100.0) | (75.0) | (58.3) | (50.0) | (71.4) | (77.8) | (68.8) |

Body weight (kg)

| | | | | | | | | |
|---|---|---|---|---|---|---|---|---|
| n | 7 | 6 | 4 | 12 | 12 | 7 | 18 | 16 |
| Mean | 79.23 | 77.25 | 73.58 | 67.99 | 80.52 | 79.23 | 73.51 | 77.30 |
| SD | 17.167 | 14.049 | 14.775 | 9.732 | 9.897 | 17.167 | 12.002 | 9.718 |
| Median | 78.80 | 75.60 | 75.90 | 71.15 | 81.00 | 78.80 | 72.20 | 79.15 |
| Minimum | 60.4 | 59.4 | 55.9 | 54.2 | 55.6 | 60.4 | 55.8 | 55.9 |
| Maximum | 109.6 | 101.4 | 86.6 | 82.8 | 93.0 | 109.6 | 101.4 | 93.1 |

BMI (kg/m²)

| | | | | | | | | |
|---|---|---|---|---|---|---|---|---|
| n | 7 | 6 | 4 | 12 | 12 | 7 | 18 | 16 |
| Mean | 25.61 | 23.59 | 25.73 | 23.60 | 26.79 | 25.61 | 24.78 | 26.51 |
| SD | 3.187 | 3.797 | 2.672 | 2.357 | 2.511 | 3.187 | 3.150 | 2.492 |
| Median | 26.44 | 23.13 | 25.55 | 23.51 | 26.87 | 26.44 | 25.03 | 27.62 |
| Minimum | 19.7 | 19.8 | 23.3 | 19.6 | 20.7 | 19.7 | 19.8 | 23.2 |
| Maximum | 29.5 | 29.5 | 28.6 | 26.7 | 29.5 | 29.5 | 29.8 | 29.4 |

Baseline measurements refer to the last measurement collected prior to the first dose. % = 100 × n/N.

### F. Pharmacokinetic and Pharmacodynamic Results

### (i) Plasma Pharmacokinetics of Formulation A

Error! Reference source not found. displays the plot of mean (+SD) plasma phloroglucinol concentrations on Day 1 by dose level on a linear and semi-logarithmic scale, respectively, for the PK Population by food status.

Following administration of Formulation A in the fed state, phloroglucinol was rapidly absorbed. 2 periods of slower decline in the mean plasma phloroglucinol concentrations were observed approximately 2 and 4 hours postdose. Systemic exposures to phloroglucinol generally increased with increasing dose across treatment groups for the high fat, low fat, and fasted states; however, relatively blunted and sustained exposures with food were evident.

Error! Reference source not found.2 displays a plot of mean (+SD) plasma trough phloroglucinol concentrations by dose level on a linear and semi-logarithmic scale, respectively, for the PK Population.

There was no noteworthy accumulation of phloroglucinol observed following administration of Formulation A each morning and evening (at nominal times of 0 and 10 hours) starting on the morning of Day 1 and continuing through the morning of Day 6. Given the shorter dosing interval of 10 hours between the morning and evening dose, trough concentrations in the evening were typically higher than the morning trough concentrations, which followed a 14-hour interval.

Table 9 summarizes plasma Formulation A PK parameters for the PK Population on Day 1. Following administration of Formulation A on Day 1, Formulation A was rapidly absorbed. In the fasted state, median Tₘₐₓ occurred at approximately 0.5 hours. In the fed state, consistent with the more blunted sustained shape of the curve, median Tₘₐₓ values ranged from 0.52 to 1.75 hours across the dosing range. The mean Cₘₐₓ increased as dose increased, with a range from 269.17 to 1201.00 ng/mL and 506.83 to 1074.83 ng/ml across the high fat and low fat treatment groups, respectively. The much higher exposures in the absence of food were apparent with mean Cₘₐₓ values of 2745.71 and 4873.33 ng/ml for the fasted 960 mg and 1280 mg treatment groups, respectively. Similar to the changes in Cₘₐₓ, the systemic exposure, as determined by AUCₜₐᵤ, followed similar trends.

TABLE 9
Summary of Plasma Formulation A Pharmacokinetics
Parameters - PK Population (Day 1)

| | | | | |
|---|---|---|---|---|
| | Cₘₐₓ | Tₘₐₓ | AUCₜₐᵤ | Cₘᵢₙ |
| Statistic | (ng/mL) | (h) | (h*ng/mL) | (ng/mL) |

Formulation A 320 mg high fat (Cohort 1a)

| | | | | |
|---|---|---|---|---|
| n | 6 | 6 | 6 | 6 |
| Mean | 269.17 | 2.00 | 879.54 | 21.55 |
| SD | 119.695 | 1.378 | 161.335 | 8.155 |
| CV % | 44.5 | 68.9 | 18.3 | 37.8 |
| Median (min, max) | 250.00 (124.0, | 1.25 (1.0, | 889.22 (620.5, | 20.60 (13.0, |
| | 451.0) | 4.0) | 1075.3) | 35.1) |
| Geometric mean | 246.71 | 1.66 | 866.25 | 20.34 |
| Geometric CV % | 49.3 | 72.5 | 19.7 | 38.3 |

Formulation A 320 mg low fat (Cohort 1b)

| | | | | |
|---|---|---|---|---|
| n | 6 | 6 | 6 | 6 |
| Mean | 506.83 | 1.05 | 920.90 | 11.82 |
| SD | 408.543 | 1.064 | 277.922 | 12.097 |
| CV % | 80.6 | 101.8 | 30.2 | 102.4 |
| Median (min, max) | 355.50 (115.0, | 0.64 (0.3, | 1014.32 (430.6, | 11.05 (0.0, |
| | 1100.0) | 3.0) | 1172.5) | 32.6) |
| Geometric mean | 370.40 | 0.69 | 875.17 | 15.93 |
| Geometric CV % | 111.5 | 129.2 | 39.1 | 54.6 |

Formulation A 640 mg high fat (Cohort 2a)

| | | | | |
|---|---|---|---|---|
| n | 6 | 6 | 3 | 6 |
| Mean | 515.83 | 2.05 | 1984.38 | 86.43 |
| SD | 209.818 | 1.699 | 1022.902 | 33.258 |
| CV % | 40.7 | 82.9 | 51.5 | 38.5 |
| Median (min, max) | 434.50 (317.0, | 1.50 (0.5, | 1395.76 (1391.9, | 87.45 (36.3, |
| | 820.0) | 4.3) | 3165.5) | 124.0) |
| Geometric mean | 483.33 | 1.45 | 1832.11 | 80.03 |
| Geometric CV % | 40.4 | 121.4 | 50.1 | 48.2 |

Formulation A 640 mg low fat (Cohort 2b)

| | | | | |
|---|---|---|---|---|
| n | 6 | 6 | 6 | 6 |
| Mean | 545.83 | 0.75 | 1652.98 | 26.57 |
| SD | 258.489 | 0.313 | 247.083 | 14.057 |
| CV % | 47.4 | 41.6 | 14.9 | 52.9 |
| Median (min, max) | 430.50 (367.0, | 0.88 (0.3, | 1609.97 (1419.0, | 30.00 (0.0, |
| | 1030.0) | 1.0) | 1981.2) | 39.4) |
| Geometric mean | 505.92 | 0.68 | 1637.78 | 31.43 |
| Geometric CV % | 42.2 | 59.8 | 14.9 | 19.2 |

Formulation A 960 mg fasted (Cohort 3c)

| | | | | |
|---|---|---|---|---|
| n | 7 | 7 | 7 | 7 |
| Mean | 2745.71 | 0.94 | 4936.49 | 38.50 |
| SD | 1222.605 | 0.740 | 1091.283 | 12.487 |
| CV % | 44.5 | 79.0 | 22.1 | 32.4 |
| Median (min, max) | 2610.00 (1380.0, | 0.52 (0.3, | 5204.76 (3157.5, | 38.50 (27.4, |
| | 4990.0) | 2.0) | 6468.9) | 64.0) |
| Geometric mean | 2530.05 | 0.73 | 4825.07 | 37.04 |
| Geometric CV % | 45.7 | 89.4 | 24.0 | 29.6 |

Formulation A 1280 mg high fat (Cohort 3a)

| | | | | |
|---|---|---|---|---|
| n | 6 | 6 | 5 | 6 |
| Mean | 1201.00 | 1.64 | 3845.94 | 105.10 |
| SD | 865.980 | 2.172 | 390.596 | 35.159 |
| CV % | 72.1 | 132.8 | 10.2 | 33.5 |
| Median (min, max) | 936.00 (620.0, | 0.64 (0.5, | 3770.53 (3296.7, | 108.00 (55.4, |
| | 2950.0) | 6.0) | 4240.5) | 153.0) |
| Geometric mean | 1036.40 | 0.99 | 3829.71 | 99.71 |
| Geometric CV % | 57.9 | 125.7 | 10.4 | 38.1 |

Formulation A 1280 mg low fat (Cohort 3b)

| | | | | |
|---|---|---|---|---|
| n | 6 | 6 | 6 | 6 |
| Mean | 1074.83 | 1.97 | 4297.78 | 81.15 |
| SD | 139.872 | 1.245 | 458.137 | 28.643 |
| CV % | 13.0 | 63.3 | 10.7 | 35.3 |
| Median (min, max) | 1145.00 (860.0, | 1.75 (0.5, | 4314.54 (3684.6, | 73.70 (53.7, |
| | 1200.0) | 4.3) | 4835.3) | 125.0) |
| Geometric mean | 1066.74 | 1.65 | 4277.18 | 77.20 |
| Geometric CV % | 13.8 | 76.9 | 10.8 | 35.3 |

Formulation A 1280 mg fasted (Cohort 3d)

| | | | | |
|---|---|---|---|---|
| n | 6 | 6 | 6 | 6 |
| Mean | 4873.33 | 0.56 | 6853.03 | 51.63 |
| SD | 1267.954 | 0.094 | 1088.679 | 19.512 |
| CV % | 26.0 | 16.8 | 15.9 | 37.8 |
| Median (min, max) | 5015.00 (2560.0, | 0.52 (0.5, | 6855.12 (5663.1, | 57.65 (19.0, |
| | 6170.0) | 0.8) | 8410.5) | 70.6) |
| Geometric mean | 4699.27 | 0.55 | 6781.18 | 47.42 |
| Geometric CV % | 32.4 | 15.0 | 16.0 | 52.9 |

Note:
*λz and λz-related parameters were listed but excluded from statistical analysis if the adjusted regression coefficient was less than 0.8 or the AUCₑₓₜᵣₐₚ exceeded 20%.
AUCₜₐᵤ was the area under the curve for the dosing interval from 0 to 10 hours. Cₘᵢₙ was the concentration at the scheduled timepoint of 10 hours post-dose, where BLQ values were imputed as 0. Geometric CV % = 100*(exp(SD²) − 1)^{0.5}, where SD was the standard deviation of the logarithmic-transformed data.

Table 1 summarizes plasma Formulation A PK parameters for the PK Population on Day 6. Given the lack of accumulation at steady-state following administration of Formulation A each morning and evening (at nominal times of 0 and 10 hours), the PK profiles on Day 6 were qualitatively and quantitatively similar to those observed following the initial dose on Day 1. t½ was similar across treatment groups with a range of 1.6 hours to 2.4 hours across all treatment groups. Mean accumulation ratios based on AUCₜₐᵤ (R_{AUCtau}) and Cₘₐₓ (R_{Cmax}) were similar and close to 1 across all treatment groups, indicating no noteworthy accumulation of Formulation A at steady-state following the dosing regimens administered.

TABLE 1
Summary of Plasma Formulation A Pharmacokinetics
Parameters - PK Population (Day 6)

| | | | | | |
|---|---|---|---|---|---|
| Cohort | Cₘₐₓ | Tₘₐₓ | t½ | AUC₀₋ₜ | AUCₜₐᵤ |
| Statistic | (ng/mL) | (h) | (h) | (h*ng/mL) | (h*ng/mL) |

Formulation A 320 mg high fat (Cohort 1a)

| | | | | | |
|---|---|---|---|---|---|
| n | 6 | 6 | 5 | 6 | 6 |
| Mean | 505.00 | 1.34 | 1.8632 | 933.10 | 913.61 |
| SD | 481.036 | 1.337 | 0.44406 | 199.046 | 207.166 |
| CV % | 95.3 | 99.5 | 23.8 | 21.3 | 22.7 |
| Median | 388.50 | 0.64 | 1.7472 | 911.33 | 866.64 |
| Minimum | 131.0 | 0.3 | 1.464 | 646.9 | 646.9 |
| Maximum | 1440.0 | 3.5 | 2.617 | 1176.0 | 1176.0 |
| Geometric | 368.32 | 0.87 | 1.8258 | 914.76 | 894.11 |
| mean | | | | | |
| Geometric | 102.5 | 134.7 | 22.1 | 22.4 | 23.1 |
| CV % | | | | | |

Formulation A 320 mg low fat (Cohort 1b)

| | | | | | |
|---|---|---|---|---|---|
| n | 6 | 6 | 6 | 6 | 6 |
| Mean | 350.83 | 1.42 | 1.9371 | 946.02 | 944.66 |
| SD | 240.055 | 0.940 | 0.43502 | 253.527 | 248.304 |
| CV % | 68.4 | 66.2 | 22.5 | 26.8 | 26.3 |
| Median | 270.00 | 1.63 | 1.8015 | 1033.67 | 1039.74 |
| Minimum | 203.0 | 0.3 | 1.560 | 486.7 | 492.9 |
| Maximum | 833.0 | 2.3 | 2.703 | 1146.5 | 1146.5 |
| Geometric | 306.36 | 1.07 | 1.9002 | 909.67 | 909.80 |
| mean | | | | | |
| Geometric | 55.5 | 116.7 | 21.2 | 33.8 | 33.0 |
| CV % | | | | | |

Formulation A 640 mg high fat (Cohort 2a)

| | | | | | |
|---|---|---|---|---|---|
| n | 6 | 6 | 6 | 6 | 6 |
| Mean | 443.67 | 1.59 | 2.0148 | 1790.96 | 1701.84 |
| SD | 140.018 | 0.841 | 0.83516 | 513.617 | 533.429 |
| CV % | 31.6 | 53.0 | 41.5 | 28.7 | 31.3 |
| Median | 378.00 | 1.75 | 1.5672 | 1605.29 | 1500.85 |
| Minimum | 321.0 | 0.5 | 1.328 | 1454.9 | 1353.9 |
| Maximum | 666.0 | 2.8 | 3.253 | 2802.7 | 2761.7 |
| Geometric | 427.18 | 1.36 | 1.8878 | 1741.18 | 1646.77 |
| mean | | | | | |
| Geometric | 30.1 | 71.9 | 39.9 | 25.1 | 27.1 |
| CV % | | | | | |

Formulation A 640 mg low fat (Cohort 2b)

| | | | | | |
|---|---|---|---|---|---|
| n | 5 | 5 | 5 | 5 | 5 |
| Mean | 648.40 | 1.65 | 1.6409 | 1710.65 | 1686.18 |
| SD | 419.429 | 2.169 | 0.27666 | 382.921 | 380.760 |
| CV % | 64.7 | 131.5 | 16.9 | 22.4 | 22.6 |
| Median | 463.00 | 0.75 | 1.5359 | 1810.35 | 1760.66 |
| Minimum | 363.0 | 0.3 | 1.357 | 1288.4 | 1294.6 |
| Maximum | 1360.0 | 5.5 | 2.019 | 2232.2 | 2212.1 |
| Geometric | 565.29 | 0.95 | 1.6228 | 1676.35 | 1652.06 |
| mean | | | | | |
| Geometric | 60.2 | 157.3 | 16.7 | 22.9 | 22.9 |
| CV % | | | | | |

Formulation A 960 mg fasted (Cohort 3c)

| | | | | | |
|---|---|---|---|---|---|
| n | 6 | 6 | 6 | 6 | 6 |
| Mean | 2791.67 | 0.55 | 2.1316 | 4608.75 | 4567.23 |
| SD | 731.640 | 0.291 | 0.28233 | 903.228 | 890.656 |
| CV % | 26.2 | 53.1 | 13.2 | 19.6 | 19.5 |
| Median | 2770.00 | 0.52 | 2.2287 | 4578.88 | 4546.94 |
| Minimum | 1950.0 | 0.3 | 1.600 | 3768.8 | 3730.2 |
| Maximum | 3960.0 | 1.0 | 2.362 | 5569.4 | 5505.5 |
| Geometric | 2713.78 | 0.48 | 2.1141 | 4534.55 | 4494.40 |
| mean | | | | | |
| Geometric | 26.5 | 61.4 | 14.6 | 20.0 | 19.9 |
| CV % | | | | | |

Formulation A 1280 mg high fat (Cohort 3a)

| | | | | | |
|---|---|---|---|---|---|
| n | 6 | 6 | 6 | 6 | 6 |
| Mean | 1293.67 | 1.30 | 2.3860 | 4096.49 | 3892.47 |
| SD | 1074.230 | 0.841 | 0.97649 | 796.635 | 922.415 |
| CV % | 83.0 | 64.6 | 40.9 | 19.4 | 23.7 |
| Median | 854.50 | 1.13 | 2.0493 | 4312.54 | 4229.30 |
| Minimum | 606.0 | 0.5 | 1.640 | 2647.4 | 2289.9 |
| Maximum | 3410.0 | 2.5 | 4.309 | 4914.8 | 4880.1 |
| Geometric | 1051.60 | 1.07 | 2.2587 | 4020.73 | 3783.84 |
| mean | | | | | |
| Geometric | 72.2 | 78.6 | 35.2 | 22.4 | 27.9 |
| CV % | | | | | |

Formulation A 1280 mg low fat (Cohort 3b)

| | | | | | |
|---|---|---|---|---|---|
| n | 6 | 6 | 6 | 6 | 6 |
| Mean | 1511.67 | 0.88 | 2.1136 | 4812.26 | 4694.96 |
| SD | 385.093 | 0.605 | 0.43820 | 724.615 | 658.244 |
| CV % | 25.5 | 68.9 | 20.7 | 15.1 | 14.0 |
| Median | 1465.00 | 0.75 | 2.1205 | 4695.91 | 4557.77 |
| Minimum | 1010.0 | 0.3 | 1.640 | 3927.8 | 3892.3 |
| Maximum | 1980.0 | 2.0 | 2.619 | 5778.4 | 5590.7 |
| Geometric | 1470.04 | 0.73 | 2.0753 | 4767.10 | 4656.96 |
| mean | | | | | |
| Geometric | 26.6 | 78.1 | 21.3 | 15.1 | 14.0 |
| CV % | | | | | |

Formulation A 1280 mg fasted (Cohort 3d)

| | | | | | |
|---|---|---|---|---|---|
| n | 6 | 6 | 6 | 6 | 6 |
| Mean | 4400.00 | 0.52 | 2.0343 | 6812.39 | 6714.19 |
| SD | 1394.317 | 0.000 | 0.43776 | 1065.723 | 1029.207 |
| CV % | 31.7 | 0.0 | 21.5 | 15.6 | 15.3 |
| Median | 3695.00 | 0.52 | 1.8891 | 7098.05 | 6978.21 |
| Minimum | 3200.0 | 0.5 | 1.641 | 5318.4 | 5303.4 |
| Maximum | 6480.0 | 0.5 | 2.893 | 8258.3 | 8191.2 |
| Geometric | 4234.01 | 0.52 | 2.0008 | 6740.54 | 6647.07 |
| mean | | | | | |
| Geometric | 30.3 | 0.0 | 19.4 | 16.2 | 15.7 |
| CV % | | | | | |

* λz and λz-related parameters were excluded from statistical analysis if the adjusted regression coefficient was less than 0.8 or the AUCₑₓₜᵣₐₚ exceeded 20%.
\hat{ } R_{AUCtau} was excluded from statistical analysis if the adjusted regression coefficient was less than 0.8 or the AUCₑₓₜᵣₐₚ exceeded 20% on Day 1. AUCₜₐᵤ was the area under the curve for the dosing interval from 0 to 10 hours. Geometric CV % = 100*(exp(SD²) − 1)^{0.5} , where SD was the standard deviation of the logarithmic-transformed data. R_{AUCtau} = accumulation ratios assessment on Day 6 only calculated as AUCₜₐᵤ on Day 6/AUCₜₐᵤ on Day 1; R_{Cmax} = accumulation ratios assessment on Day 6 only calculated as Cₘₐₓ on Day 6/Cₘₐₓ on Day 1.

TABLE 2
Summary of Plasma Formulation A Pharmacokinetics
Parameters - PK Population (Day 6) (continuted)

| | | | | | |
|---|---|---|---|---|---|
| | Cₘₐₓ | Tₘₐₓ | t½ | AUC₀₋ₜ | AUCₜₐᵤ |
| Statistic | (ng/mL) | (h) | (h) | (h*ng/mL) | (h*ng/mL) |

Formulation A 320 mg high fat (Cohort 1a)

| | | | | | |
|---|---|---|---|---|---|
| n | 6 | 6 | 5 | 6 | 6 |
| Mean | 505.00 | 1.34 | 1.8632 | 933.10 | 913.61 |
| SD | 481.036 | 1.337 | 0.44406 | 199.046 | 207.166 |
| CV % | 95.3 | 99.5 | 23.8 | 21.3 | 22.7 |
| Median | 388.50 | 0.64 | 1.7472 | 911.33 | 866.64 |
| Minimum | 131.0 | 0.3 | 1.464 | 646.9 | 646.9 |
| Maximum | 1440.0 | 3.5 | 2.617 | 1176.0 | 1176.0 |
| Geometric | 368.32 | 0.87 | 1.8258 | 914.76 | 894.11 |
| mean | | | | | |
| Geometric | 102.5 | 134.7 | 22.1 | 22.4 | 23.1 |
| CV % | | | | | |

Formulation A 320 mg low fat (Cohort 1b)

| | | | | | |
|---|---|---|---|---|---|
| n | 6 | 6 | 6 | 6 | 6 |
| Mean | 350.83 | 1.42 | 1.9371 | 946.02 | 944.66 |
| SD | 240.055 | 0.940 | 0.43502 | 253.527 | 248.304 |
| CV % | 68.4 | 66.2 | 22.5 | 26.8 | 26.3 |
| Median | 270.00 | 1.63 | 1.8015 | 1033.67 | 1039.74 |
| Minimum | 203.0 | 0.3 | 1.560 | 486.7 | 492.9 |
| Maximum | 833.0 | 2.3 | 2.703 | 1146.5 | 1146.5 |
| Geometric | 306.36 | 1.07 | 1.9002 | 909.67 | 909.80 |
| mean | | | | | |
| Geometric | 55.5 | 116.7 | 21.2 | 33.8 | 33.0 |
| CV % | | | | | |

Formulation A 640 mg high fat (Cohort 2a)

| | | | | | |
|---|---|---|---|---|---|
| n | 6 | 6 | 6 | 6 | 6 |
| Mean | 443.67 | 1.59 | 2.0148 | 1790.96 | 1701.84 |
| SD | 140.018 | 0.841 | 0.83516 | 513.617 | 533.429 |
| CV % | 31.6 | 53.0 | 41.5 | 28.7 | 31.3 |
| Median | 378.00 | 1.75 | 1.5672 | 1605.29 | 1500.85 |
| Minimum | 321.0 | 0.5 | 1.328 | 1454.9 | 1353.9 |
| Maximum | 666.0 | 2.8 | 3.253 | 2802.7 | 2761.7 |
| Geometric | 427.18 | 1.36 | 1.8878 | 1741.18 | 1646.77 |
| mean | | | | | |
| Geometric | 30.1 | 71.9 | 39.9 | 25.1 | 27.1 |
| CV % | | | | | |

Formulation A 640 mg low fat (Cohort 2b)

| | | | | | |
|---|---|---|---|---|---|
| n | 5 | 5 | 5 | 5 | 5 |
| Mean | 648.40 | 1.65 | 1.6409 | 1710.65 | 1686.18 |
| SD | 419.429 | 2.169 | 0.27666 | 382.921 | 380.760 |
| CV % | 64.7 | 131.5 | 16.9 | 22.4 | 22.6 |
| Median | 463.00 | 0.75 | 1.5359 | 1810.35 | 1760.66 |
| Minimum | 363.0 | 0.3 | 1.357 | 1288.4 | 1294.6 |
| Maximum | 1360.0 | 5.5 | 2.019 | 2232.2 | 2212.1 |
| Geometric | 565.29 | 0.95 | 1.6228 | 1676.35 | 1652.06 |
| mean | | | | | |
| Geometric | 60.2 | 157.3 | 16.7 | 22.9 | 22.9 |
| CV % | | | | | |

Formulation A 960 mg fasted (Cohort 3c)

| | | | | | |
|---|---|---|---|---|---|
| n | 6 | 6 | 6 | 6 | 6 |
| Mean | 2791.67 | 0.55 | 2.1316 | 4608.75 | 4567.23 |
| SD | 731.640 | 0.291 | 0.28233 | 903.228 | 890.656 |
| CV % | 26.2 | 53.1 | 13.2 | 19.6 | 19.5 |
| Median | 2770.00 | 0.52 | 2.2287 | 4578.88 | 4546.94 |
| Minimum | 1950.0 | 0.3 | 1.600 | 3768.8 | 3730.2 |
| Maximum | 3960.0 | 1.0 | 2.362 | 5569.4 | 5505.5 |
| Geometric | 2713.78 | 0.48 | 2.1141 | 4534.55 | 4494.40 |
| mean | | | | | |
| Geometric | 26.5 | 61.4 | 14.6 | 20.0 | 19.9 |
| CV % | | | | | |

Formulation A 1280 mg high fat (Cohort 3a)

| | | | | | |
|---|---|---|---|---|---|
| n | 6 | 6 | 6 | 6 | 6 |
| Mean | 1293.67 | 1.30 | 2.3860 | 4096.49 | 3892.47 |
| SD | 1074.230 | 0.841 | 0.97649 | 796.635 | 922.415 |
| CV % | 83.0 | 64.6 | 40.9 | 19.4 | 23.7 |
| Median | 854.50 | 1.13 | 2.0493 | 4312.54 | 4229.30 |
| Minimum | 606.0 | 0.5 | 1.640 | 2647.4 | 2289.9 |
| Maximum | 3410.0 | 2.5 | 4.309 | 4914.8 | 4880.1 |
| Geometric | 1051.60 | 1.07 | 2.2587 | 4020.73 | 3783.84 |
| mean | | | | | |
| Geometric | 72.2 | 78.6 | 35.2 | 22.4 | 27.9 |
| CV % | | | | | |

Formulation A 1280 mg low fat (Cohort 3b)

| | | | | | |
|---|---|---|---|---|---|
| n | 6 | 6 | 6 | 6 | 6 |
| Mean | 1511.67 | 0.88 | 2.1136 | 4812.26 | 4694.96 |
| SD | 385.093 | 0.605 | 0.43820 | 724.615 | 658.244 |
| CV % | 25.5 | 68.9 | 20.7 | 15.1 | 14.0 |
| Median | 1465.00 | 0.75 | 2.1205 | 4695.91 | 4557.77 |
| Minimum | 1010.0 | 0.3 | 1.640 | 3927.8 | 3892.3 |
| Maximum | 1980.0 | 2.0 | 2.619 | 5778.4 | 5590.7 |
| Geometric | 1470.04 | 0.73 | 2.0753 | 4767.10 | 4656.96 |
| mean | | | | | |
| Geometric | 26.6 | 78.1 | 21.3 | 15.1 | 14.0 |
| CV % | | | | | |

Formulation A 1280 mg fasted (Cohort 3d)

| | | | | | |
|---|---|---|---|---|---|
| n | 6 | 6 | 6 | 6 | 6 |
| Mean | 4400.00 | 0.52 | 2.0343 | 6812.39 | 6714.19 |
| SD | 1394.317 | 0.000 | 0.43776 | 1065.723 | 1029.207 |
| CV % | 31.7 | 0.0 | 21.5 | 15.6 | 15.3 |
| Median | 3695.00 | 0.52 | 1.8891 | 7098.05 | 6978.21 |
| Minimum | 3200.0 | 0.5 | 1.641 | 5318.4 | 5303.4 |
| Maximum | 6480.0 | 0.5 | 2.893 | 8258.3 | 8191.2 |
| Geometric | 4234.01 | 0.52 | 2.0008 | 6740.54 | 6647.07 |
| mean | | | | | |
| Geometric | 30.3 | 0.0 | 19.4 | 16.2 | 15.7 |
| CV % | | | | | |

* λz and λz-related parameters were excluded from statistical analysis if the adjusted regression coefficient was less than 0.8 or the AUCₑₓₜᵣₐₚ exceeded 20%.
\hat{ } R_{AUCtau} was excluded from statistical analysis if the adjusted regression coefficient was less than 0.8 or the AUCₑₓₜᵣₐₚ exceeded 20% on Day 1. AUCₜₐᵤ was the area under the curve for the dosing interval from 0 to 10 hours. Geometric CV % = 100*(exp(SD²) − 1)^{0.5} , where SD was the standard deviation of the logarithmic-transformed data. R_{AUCtau} = accumulation ratios assessment on Day 6 only calculated as AUCₜₐᵤ on Day 6/AUCₜₐᵤ on Day 1; R_{Cmax} = accumulation ratios assessment on Day 6 only calculated as Cₘₐₓ on Day 6/Cₘₐₓ on Day 1.

TABLE 3
Summary of Plasma Formulation A Pharmacokinetics
Parameters - PK Population (Day 6) (continuted)

| | | | | | |
|---|---|---|---|---|---|
| Cohort | AUC_{0-inf} | | | CL/F | Vd/F |
| Statistic | (h*ng/mL) | R_{Cmax} | R_{AUCtau} | (L/h) | (L) |

Formulation A 320 mg high fat (Cohort 1a)

| | | | | | |
|---|---|---|---|---|---|
| n | 5 | 6 | 6 | 5 | 5 |
| Mean | 970.64 | 1.6396 | 1.0376 | 344.98 | 914.41 |
| SD | 221.638 | 0.85385 | 0.11594 | 84.948 | 246.307 |
| CV % | 22.8 | 52.1 | 11.2 | 24.6 | 26.9 |
| Median | 959.76 | 1.2435 | 1.0369 | 333.42 | 957.70 |
| Minimum | 677.9 | 1.022 | 0.871 | 265.0 | 628.7 |
| Maximum | 1207.6 | 3.193 | 1.216 | 472.0 | 1258.6 |
| Geometric | 949.41 | 1.4929 | 1.0322 | 337.05 | 887.83 |
| mean | | | | | |
| Geometric | 24.2 | 47.6 | 11.2 | 24.2 | 27.8 |
| CV % | | | | | |

Formulation A 320 mg low fat (Cohort 1b)

| | | | | | |
|---|---|---|---|---|---|
| n | 6 | 6 | 6 | 6 | 6 |
| Mean | 979.34 | 0.9776 | 1.0412 | 355.40 | 966.01 |
| SD | 255.150 | 0.58472 | 0.06428 | 134.827 | 298.969 |
| CV % | 26.1 | 59.8 | 6.2 | 37.9 | 30.9 |
| Median | 1063.87 | 0.8574 | 1.0353 | 301.53 | 924.45 |
| Minimum | 515.5 | 0.317 | 0.976 | 271.4 | 662.5 |
| Maximum | 1179.2 | 1.852 | 1.145 | 620.8 | 1437.5 |
| Geometric | 944.03 | 0.8271 | 1.0396 | 338.97 | 929.28 |
| mean | | | | | |
| Geometric | 32.5 | 73.5 | 6.1 | 32.5 | 31.0 |
| CV % | | | | | |

Formulation A 640 mg high fat (Cohort 2a)

| | | | | | |
|---|---|---|---|---|---|
| n | 6 | 6 | 3 | 6 | 6 |
| Mean | 1889.36 | 0.9510 | 0.9969 | 354.01 | 1035.84 |
| SD | 488.385 | 0.39969 | 0.12058 | 72.114 | 487.770 |
| CV % | 25.8 | 42.0 | 12.1 | 20.4 | 47.1 |
| Median | 1767.36 | 0.8593 | 1.0049 | 363.31 | 861.11 |
| Minimum | 1491.6 | 0.475 | 0.872 | 226.1 | 481.0 |
| Maximum | 2831.2 | 1.610 | 1.113 | 429.1 | 1802.6 |
| Geometric | 1844.91 | 0.8838 | 0.9919 | 346.90 | 944.80 |
| mean | | | | | |
| Geometric | 23.3 | 44.2 | 12.3 | 23.3 | 50.0 |
| CV % | | | | | |

Formulation A 640 mg low fat (Cohort 2b)

| | | | | | |
|---|---|---|---|---|---|
| n | 5 | 5 | 5 | 5 | 5 |
| Mean | 1744.70 | 1.1383 | 0.9875 | 381.44 | 906.04 |
| SD | 382.921 | 0.56639 | 0.08619 | 84.237 | 265.759 |
| CV % | 21.9 | 49.8 | 8.7 | 22.1 | 29.3 |
| Median | 1854.31 | 0.9865 | 0.9471 | 345.14 | 914.01 |
| Minimum | 1312.9 | 0.670 | 0.912 | 282.4 | 593.6 |
| Maximum | 2266.0 | 2.105 | 1.117 | 487.5 | 1307.6 |
| Geometric | 1711.02 | 1.0479 | 0.9845 | 374.05 | 875.70 |
| mean | | | | | |
| Geometric | 22.4 | 45.7 | 8.5 | 22.4 | 29.8 |
| CV % | | | | | |

Formulation A 960 mg fasted (Cohort 3c)

| | | | | | |
|---|---|---|---|---|---|
| n | 6 | 6 | 6 | 6 | 6 |
| Mean | 4659.20 | 1.2398 | 0.9836 | 212.83 | 652.36 |
| SD | 913.421 | 0.61209 | 0.11619 | 41.581 | 152.081 |
| CV % | 19.6 | 49.4 | 11.8 | 19.5 | 23.3 |
| Median | 4612.35 | 1.2416 | 0.9784 | 214.18 | 568.54 |
| Minimum | 3816.6 | 0.582 | 0.868 | 170.0 | 527.3 |
| Maximum | 5645.7 | 2.200 | 1.181 | 251.5 | 853.6 |
| Geometric | 4584.19 | 1.1119 | 0.9781 | 209.42 | 638.71 |
| mean | | | | | |
| Geometric | 20.0 | 56.0 | 11.5 | 20.0 | 22.4 |
| CV % | | | | | |

Formulation A 1280 mg high fat (Cohort 3a)

| | | | | | |
|---|---|---|---|---|---|
| n | 6 | 6 | 5 | 6 | 6 |
| Mean | 4179.78 | 1.0573 | 1.0973 | 316.14 | 1157.30 |
| SD | 735.466 | 0.35255 | 0.11904 | 68.294 | 799.466 |
| CV % | 17.6 | 33.3 | 10.8 | 21.6 | 69.1 |
| Median | 4381.84 | 0.9650 | 1.0263 | 292.12 | 864.15 |
| Minimum | 2865.0 | 0.686 | 1.016 | 258.3 | 687.7 |
| Maximum | 4955.8 | 1.706 | 1.294 | 446.8 | 2777.7 |
| Geometric | 4118.31 | 1.0147 | 1.0924 | 310.81 | 1012.79 |
| mean | | | | | |
| Geometric | 19.7 | 31.4 | 10.4 | 19.7 | 54.2 |
| CV % | | | | | |

Formulation A 1280 mg low fat (Cohort 3b)

| | | | | | |
|---|---|---|---|---|---|
| n | 6 | 6 | 6 | 6 | 6 |
| Mean | 4854.38 | 1.4372 | 1.0916 | 268.63 | 800.23 |
| SD | 725.630 | 0.48702 | 0.08422 | 39.932 | 81.168 |
| CV % | 14.9 | 33.9 | 7.7 | 14.9 | 10.1 |
| Median | 4751.89 | 1.2427 | 1.1057 | 270.28 | 774.41 |
| Minimum | 3957.2 | 1.052 | 0.949 | 220.1 | 700.3 |
| Maximum | 5816.6 | 2.302 | 1.179 | 323.5 | 918.5 |
| Geometric | 4809.38 | 1.3781 | 1.0888 | 266.15 | 796.87 |
| mean | | | | | |
| Geometric | 15.0 | 31.5 | 8.0 | 15.0 | 10.0 |
| CV % | | | | | |

Formulation A 1280 mg fasted (Cohort 3d)

| | | | | | |
|---|---|---|---|---|---|
| n | 6 | 6 | 6 | 6 | 6 |
| Mean | 6860.99 | 0.9509 | 0.9814 | 190.65 | 554.20 |
| SD | 1074.765 | 0.33372 | 0.05270 | 31.577 | 117.844 |
| CV % | 15.7 | 35.1 | 5.4 | 16.6 | 21.3 |
| Median | 7160.73 | 0.9305 | 0.9962 | 178.77 | 512.56 |
| Minimum | 5353.8 | 0.587 | 0.904 | 153.9 | 417.7 |
| Maximum | 8319.4 | 1.359 | 1.035 | 239.1 | 739.0 |
| Geometric | 6788.40 | 0.9010 | 0.9802 | 188.56 | 544.27 |
| mean | | | | | |
| Geometric | 16.3 | 37.6 | 5.4 | 16.3 | 20.9 |
| CV % | | | | | |

* λz and λz-related parameters were excluded from statistical analysis if the adjusted regression coefficient was less than 0.8 or the AUCₑₓₜᵣₐₚ exceeded 20%.
\hat{ } R_{AUCtau} was excluded from statistical analysis if the adjusted regression coefficient was less than 0.8 or the AUCₑₓₜᵣₐₚ exceeded 20% on Day 1. AUCₜₐᵤ was the area under the curve for the dosing interval from 0 to 10 hours. Geometric CV % = 100*(exp(SD²) − 1)^{0.5} , where SD was the standard deviation of the logarithmic-transformed data. R_{AUCtau} = accumulation ratios assessment on Day 6 only calculated as AUCₜₐᵤ on Day 6/AUCₜₐᵤ on Day 1; R_{Cmax} = accumulation ratios assessment on Day 6 only calculated as Cₘₐₓ on Day 6/Cₘₐₓ on Day 1.

### (ii) Dose-Proportionality of Formulation A

The dose proportionality results were assessed using the Smith criteria (90% CI: 0.839 to 1.161) and the Hummel criteria (90% CI: 0.50 to 1.50) across the full dose range for both the high fat and low fat state. Overall, no gross trends indicating deviation from dose proportionality were observed across the dose range studied.

Table 4 summarizes the power model analysis of dose proportionality of Formulation A for the PK Evaluable Population on Day 1 for the 320 mg, 640 mg, and 1280 mg high and low fat Formulation A treatment groups.

On Day 1, the estimated slopes of the dose versus exposure relationships (with 90% CIs) across the high fat 320 mg, 640 mg, and 1280 mg Formulation A treatment groups were 1.035 (0.705, 1.365) for Cₘₐₓ, and 1.072 (0.887, 1.258) for AUCₜₐᵤ. Both parameters assessed (Cₘₐₓ and AUCₜₐᵤ) met the Hummel criteria, but not the more stringent Smith criteria. For the low fat 320 mg, 640 mg, and 1280 mg Formulation A treatment groups on Day 1, the estimated slopes of the dose versus exposure relationships (with 90% CIs) were 0.763 (0.351, 1.175) for Cₘₐₓ, and 1.145 (0.964, 1.325) for AUCₜₐᵤ. AUCₜₐᵤ met the Hummel criteria, but not the Smith criteria.

TABLE 4
Power Model Analysis of Dose Proportionality of Formulation
A - PK Evaluable Population - Day 1 (High Fat and Low Fat)

Dose Level

| | | | |
|---|---|---|---|
| PK Parameter (PK Unit) | 320 mg | 640 mg | 1280 mg |
| Statistic | (N = 6) | (N = 6) | (N = 6) |

High Fat Formulation A
Cₘₐₓ (ng/mL)

| | | | |
|---|---|---|---|
| n | 6 | 6 | 6 |
| GM | 246.71 | 483.33 | 1036.40 |
| CV % for GM | 49.3 | 40.4 | 57.9 |

Dose proportionality for Cₘₐₓ

| | |
|---|---|
| n | 18 |
| Slope estimate | 1.035 |
| Standard error | 0.1891 |
| 90% CI | (0.705, 1.365) |

AUCₜₐᵤ (h*ng/mL)

| | | | |
|---|---|---|---|
| n | 6 | 3 | 5 |
| GM | 866.25 | 1832.11 | 3829.71 |
| CV % for GM | 19.7 | 50.1 | 10.4 |

Dose proportionality for AUCₜₐᵤ

| | |
|---|---|
| n | 14 |
| Slope estimate | 1.072 |
| Standard error | 0.1041 |
| 90% CI | (0.887, 1.258) |

Low Fat Formulation A
Cₘₐₓ (ng/mL)

| | | | |
|---|---|---|---|
| n | 6 | 6 | 6 |
| GM | 370.40 | 505.92 | 1066.74 |
| CV % for GM | 111.5 | 42.2 | 13.8 |

Dose proportionality for Cₘₐₓ

| | |
|---|---|
| n | 18 |
| Slope estimate | 0.763 |
| Standard error | 0.2361 |
| 90% CI | (0.351, 1.175) |

AUCₜₐᵤ (h*ng/mL)

| | | | |
|---|---|---|---|
| n | 6 | 6 | 6 |
| GM | 875.17 | 1637.78 | 4277.18 |
| CV % for GM | 39.1 | 14.9 | 10.8 |

Dose proportionality for AUCₜₐᵤ

| | |
|---|---|
| n | 18 |
| Slope estimate | 1.145 |
| Standard error | 0.1036 |
| 90% CI | (0.964, 1.325) |

The power model was fitted by REML using SAS ® Proc Mixed. The Smith and Hummel critical regions for dose proportionality CI are (0.839, 1.161) and (0.5, 1.5), respectively.

Table 5 summarizes the power model analysis of dose proportionality of Formulation A for the PK Evaluable Population on Day 6 for the 320 mg, 620 mg, and 1280 mg high and low fat Formulation A treatment groups.

On Day 6, the estimated slopes of the dose versus exposure relationships (with 90% CIs) across the high fat 320 mg, 640 mg, and 1280 mg Formulation A treatment groups were 0.757 (0.287, 1.227) for Cₘₐₓ, 1.062 (0.896, 1.227) for AUC_{0-inf}, and 1.068 (0.902, 1.234) for AUC₀₋ₜ. Only AUC₀₋ₜ and AUC_{0-inf} met the Hummel criteria, but not the Smith criteria. On Day 6, the estimated slopes of the dose versus exposure relationships (with 90% CIs) across the low fat 320 mg, 640 mg, and 1280 mg Formulation A treatment groups were 1.131 (0.803, 1.459) for Cₘₐₓ, 1.174 (0.988, 1.361) for AUC_{0-inf}, and 1.195 (1.004, 1.385) for AUC₀₋ₜ. All the parameters assessed (Cₘₐₓ, AUC₀₋ₜ, and AUC_{0-inf}) met the Hummel criteria, but not the Smith criteria.

TABLE 5
Power Model Analysis of Dose Proportionality of Formulation
A - PK Evaluable Population - Day 6 (High Fat and Low Fat)

| | Dose Level | | |
|---|---|---|---|
| PK Parameter (PK Unit) | 320 mg | 640 mg | 1280 mg |
| Statistic | (N = 6) | (N = 6) | (N = 6) |

High Fat Formulation A
Cₘₐₓ (ng/mL)

| | | | |
|---|---|---|---|
| n | 6 | 6 | 6 |
| GM | 368.32 | 427.18 | 1051.60 |
| CV % for GM | 102.5 | 30.1 | 72.2 |

Dose proportionality for Cₘₐₓ

| | |
|---|---|
| n | 18 |
| Slope estimate | 0.757 |
| Standard error | 0.2693 |
| 90% CI | (0.287, 1.227) |

AUC_{0-inf} (h*ng/mL)

| | | | |
|---|---|---|---|
| n | 5 | 6 | 6 |
| GM | 949.41 | 1844.91 | 4118.31 |
| CV % for GM | 24.2 | 23.3 | 19.7 |

Dose proportionality for AUC_{0-inf}

| | |
|---|---|
| n | 17 |
| Slope estimate | 1.062 |
| Standard error | 0.0943 |
| 90% CI | (0.896, 1.227) |

AUC₀₋ₜ (h*ng/mL)

| | | | |
|---|---|---|---|
| n | 6 | 6 | 6 |
| GM | 914.76 | 1741.18 | 4020.73 |
| CV % for GM | 22.4 | 25.1 | 22.4 |

Dose proportionality for AUC₀₋ₜ

| | |
|---|---|
| n | 18 |
| Slope estimate | 1.068 |
| Standard error | 0.0950 |
| 90% CI | (0.902, 1.234) |

Low Fat Formulation A
Cₘₐₓ (ng/mL)

| | | | |
|---|---|---|---|
| n | 6 | 5 | 6 |
| GM | 306.36 | 565.29 | 1470.04 |
| CV % for GM | 55.5 | 60.2 | 26.6 |

Dose proportionality for Cₘₐₓ

| | |
|---|---|
| n | 17 |
| Slope estimate | 1.131 |
| Standard error | 0.1870 |
| 90% CI | (0.803, 1.459) |

AUC_{0-inf} (h*ng/mL)

| | | | |
|---|---|---|---|
| n | 6 | 5 | 6 |
| GM | 944.03 | 1711.02 | 4809.38 |
| CV % for GM | 32.5 | 22.4 | 15.0 |

Dose proportionality for AUC_{0-inf}

| | |
|---|---|
| n | 17 |
| Slope estimate | 1.174 |
| Standard error | 0.1065 |
| 90% CI | (0.988, 1.361) |

AUC₀₋ₜ (h*ng/mL)

| | | | |
|---|---|---|---|
| n | 6 | 5 | 6 |
| GM | 909.67 | 1676.35 | 4767.10 |
| CV % for GM | 33.8 | 22.9 | 15.1 |

Dose proportionality for AUC₀₋ₜ

| | |
|---|---|
| n | 17 |
| Slope estimate | 1.195 |
| Standard error | 0.1087 |
| 90% CI | (1.004, 1.385) |

The power model was fitted by REML using SAS ® Proc Mixed. The Smith and Hummel critical regions for dose proportionality CI are (0.839, 1.161) and (0.5, 1.5), respectively.

### (III) URINE PHARMACOKINETICS OF FORMULATION A

Table 13 summarizes urine Formulation A PK parameters on Day 1 for the PK Population. Consistent with the rapid clearance of Formulation A from the plasma, Formulation A was rapidly excreted in the urine. The PK profile of Formulation A in urine on Day 1 generally followed similar dose-dependent trends as the corresponding plasma PK profiles for all dosing regimens studied.

TABLE 13
Formulation A Pharmacokinetics Parameters Day 1 - PK Population

| PK | | High Fat | | | Low Fat | | | Fasted | |
|---|---|---|---|---|---|---|---|---|---|
| Parameter | | Cohort | Cohort | Cohort | Cohort | Cohort | Cohort | Cohort | Cohort |
| (Unit) | Statistic | 1a/320 mg | 2a/640 mg | 3a/1280 mg | 1b/320 mg | 2b/640 mg | 3b/1280 mg | 3c/960 mg | 3d/1280 mg |

Amount excreted

| | | | | | | | | | |
|---|---|---|---|---|---|---|---|---|---|
| Ae | n | 6 | 6 | 6 | 6 | 6 | 6 | 7 | 6 |
| (0-2 h) | Mean | 326.1 | 661.6 | 1668.0 | 403.9 | 971.7 | 1261.5 | 3556.5 | 4567.4 |
| (ug) | (SD) | (203.72) | (497.19) | (697.74) | (314.20) | (480.11) | (385.36) | (2249.85) | (987.36) |
| | Geo. | 83.3 | 203.0 | 47.0 | 85.8 | 41.9 | 38.2 | 78.6 | 21.1 |
| | CV % | | | | | | | | |
| Ae | n | 6 | 6 | 6 | 6 | 6 | 6 | 7 | 6 |
| (0-4 h) | Mean | 735.2 | 1728.1 | 3426.6 | 661.4 | 1414.3 | 3107.9 | 5903.9 | 6082.4 |
| (ug) | (SD) | (321.92) | (499.54) | (1641.96) | (427.76) | (589.02) | (1045.27) | (3118.35) | (1492.47) |
| | Geo. | 53.3 | 34.5 | 48.4 | 75.8 | 37.5 | 37.5 | 58.1 | 25.2 |
| | CV % | | | | | | | | |
| Ae | n | 6 | 6 | 6 | 6 | 6 | 6 | 7 | 6 |
| (0-6 h) | Mean | 1006.3 | 2160.9 | 4434.4 | 852.5 | 1876.0 | 4145.3 | 6907.7 | 7232.5 |
| (ug) | (SD) | (377.63) | (591.48) | (2157.09) | (456.24) | (539.03) | (1593.22) | (3353.83) | (1656.64) |
| | Geo. | 44.3 | 35.4 | 46.2 | 63.5 | 28.4 | 43.8 | 52.0 | 23.2 |
| | CV % | | | | | | | | |
| Ae | n | 6 | 6 | 6 | 6 | 6 | 6 | 7 | 6 |
| (0-10 h) | Mean | 1307.4 | 3067.2 | 5962.4 | 1069.1 | 2378.4 | 5201.7 | 7464.0 | 7836.0 |
| (ug) | (SD) | (313.23) | (627.86) | (2739.97) | (577.37) | (395.75) | (1678.20) | (3440.08) | (1800.38) |
| | Geo. | 26.8 | 21.9 | 47.3 | 69.6 | 16.7 | 38.1 | 49.9 | 23.6 |
| | CV % | | | | | | | | |
| Ae | n | 6 | 6 | 6 | 6 | 6 | 6 | 7 | 6 |
| (0-12 h) | Mean | 1797.5 | 4068.5 | 7487.6 | 1377.7 | 3107.2 | 6264.9 | 10574.8 | 9771.6 |
| (ug) | (SD) | (937.30) | (1162.38) | (2805.64) | (522.00) | (811.54) | (2151.94) | (5083.79) | (2478.50) |
| | Geo. | 50.2 | 30.9 | 35.9 | 46.4 | 24.8 | 41.3 | 53.6 | 26.9 |
| | CV % | | | | | | | | |
| Ae | n | 6 | 6 | 6 | 6 | 6 | 6 | 7 | 6 |
| (0-14 h) | Mean | 2303.1 | 5224.9 | 9641.5 | 1749.2 | 3945.9 | 8518.7 | 12306.8 | 11927.7 |
| (ug) | (SD) | (1034.12) | (1376.01) | (4000.52) | (741.16) | (815.28) | (3603.20) | (5951.10) | (3409.59) |
| | Geo. | 44.9 | 29.4 | 38.1 | 50.2 | 21.2 | 53.1 | 52.9 | 28.1 |
| | CV % | | | | | | | | |
| Ae | n | 6 | 6 | 6 | 6 | 6 | 6 | 7 | 6 |
| (0-16 h) | Mean | 2553.7 | 5811.3 | 10784.7 | 1978.8 | 4225.5 | 9758.6 | 13074.0 | 12534.2 |
| (ug) | (SD) | (1045.05) | (1308.34) | (4484.73) | (865.12) | (755.41) | (4052.19) | (6175.12) | (3605.06) |
| | Geo. | 42.9 | 23.8 | 41.1 | 48.5 | 18.4 | 51.9 | 51.9 | 27.9 |
| | CV % | | | | | | | | |
| Ae | n | 6 | 6 | 6 | 6 | 6 | 6 | 7 | 6 |
| (0-24 h) | Mean | 2799.9 | 6804.6 | 12337.2 | 2267.2 | 4657.4 | 11387.7 | 13658.8 | 13270.1 |
| (ug) | (SD) | (971.78) | (966.45) | (4711.27) | (902.80) | (710.34) | (3949.24) | (6292.62) | (3755.87) |
| | Geo. | 35.0 | 14.8 | 39.2 | 45.1 | 16.1 | 40.7 | 52.0 | 28.1 |
| | CV % | | | | | | | | |

Fraction excreted

| | | | | | | | | | |
|---|---|---|---|---|---|---|---|---|---|
| Fe | n | 6 | 6 | 6 | 6 | 6 | 6 | 7 | 6 |
| (0-2 h) | Mean | 0.1019 | 0.1034 | 0.1303 | 0.1262 | 0.1518 | 0.0986 | 0.3705 | 0.3568 |
| (%) | (SD) | (0.06366) | (0.07769) | (0.05451) | (0.09819) | (0.07502) | (0.03011) | (0.23436) | (0.07714) |
| | Geo. | 83.3 | 203.0 | 47.0 | 85.8 | 41.9 | 38.2 | 78.6 | 21.1 |
| | CV % | | | | | | | | |
| Fe | n | 6 | 6 | 6 | 6 | 6 | 6 | 7 | 6 |
| (0-4 h) | Mean | 0.2298 | 0.2700 | 0.2677 | 0.2067 | 0.2210 | 0.2428 | 0.6150 | 0.4752 |
| (%) | (SD) | (0.10060) | (0.07805) | (0.12828) | (0.13367) | (0.09203) | (0.08166) | (0.32483) | (0.11660) |
| | Geo. | 53.3 | 34.5 | 48.4 | 75.8 | 37.5 | 37.5 | 58.1 | 25.2 |
| | CV % | | | | | | | | |
| Fe | n | 6 | 6 | 6 | 6 | 6 | 6 | 7 | 6 |
| (0-6 h) | Mean | 0.3145 | 0.3376 | 0.3464 | 0.2664 | 0.2931 | 0.3239 | 0.7196 | 0.5650 |
| (%) | (SD) | (0.11801) | (0.09242) | (0.16852) | (0.14258) | (0.08422) | (0.12447) | (0.34936) | (0.12942) |
| | Geo. | 44.3 | 35.4 | 46.2 | 63.5 | 28.4 | 43.8 | 52.0 | 23.2 |
| | CV % | | | | | | | | |
| Fe | n | 6 | 6 | 6 | 6 | 6 | 6 | 7 | 6 |
| (0-10 h) | Mean | 0.4086 | 0.4793 | 0.4658 | 0.3341 | 0.3716 | 0.4064 | 0.7775 | 0.6122 |
| (%) | (SD) | (0.09788) | (0.09810) | (0.21406) | (0.18043) | (0.06184) | (0.13111) | (0.35834) | (0.14065) |
| | Geo. | 26.8 | 21.9 | 47.3 | 69.6 | 16.7 | 38.1 | 49.9 | 23.6 |
| | CV % | | | | | | | | |
| Fe | n | 6 | 6 | 6 | 6 | 6 | 6 | 7 | 6 |
| (0-12 h) | Mean | 0.2809 | 0.3179 | 0.2925 | 0.2153 | 0.2427 | 0.2447 | 0.5508 | 0.3817 |
| (%) | (SD) | (0.14645) | (0.09081) | (0.10960) | (0.08156) | (0.06340) | (0.08406) | (0.26478) | (0.09682) |
| | Geo. | 50.2 | 30.9 | 35.9 | 46.4 | 24.8 | 41.3 | 53.6 | 26.9 |
| | CV % | | | | | | | | |
| Fe | n | 6 | 6 | 6 | 6 | 6 | 6 | 7 | 6 |
| (0-14 h) | Mean | 0.3599 | 0.4082 | 0.3766 | 0.2733 | 0.3083 | 0.3328 | 0.6410 | 0.4659 |
| (%) | (SD) | (0.16158) | (0.10750) | (0.15627) | (0.11581) | (0.06369) | (0.14075) | (0.30995) | (0.13319) |
| | Geo. | 44.9 | 29.4 | 38.1 | 50.2 | 21.2 | 53.1 | 52.9 | 28.1 |
| | CV % | | | | | | | | |

Error! Reference source not found.. Summary of Urine Formulation A Pharmacokinetics
Parameters Day 1 - PK Population (Continued)

| | | High Fat | | | Low Fat | | | Fasted | |
|---|---|---|---|---|---|---|---|---|---|
| | | Cohort | Cohort | Cohort | Cohort | Cohort | Cohort | Cohort | Cohort |
| PK | | 1a/320 mg | 2a/640 mg | 3a/1280 mg | 1b/320 mg | 2b/640 mg | 3b/1280 mg | 3c/960 mg | 3d/1280 mg |
| Parameter | Statistic | (N = 6) | (N = 6) | (N = 6) | (N = 6) | (N = 6) | (N = 6) | (N = 7) | (N = 6) |
| Fe | n | 6 | 6 | 6 | 6 | 6 | 6 | 7 | 6 |
| (0-16 h) | Mean | 0.3990 | 0.4540 | 0.4213 | 0.3092 | 0.3301 | 0.3812 | 0.6809 | 0.4896 |
| (%) | (SD) | (0.16329) | (0.10221) | (0.17518) | (0.13517) | (0.05902) | (0.15829) | (0.32162) | (0.14082) |
| | Geo. | 42.9 | 23.8 | 41.1 | 48.5 | 18.4 | 51.9 | 51.9 | 27.9 |
| | CV % | | | | | | | | |
| Fe | n | 6 | 6 | 6 | 6 | 6 | 6 | 7 | 6 |
| (0-24 h) | Mean | 0.4375 | 0.5316 | 0.4819 | 0.3543 | 0.3639 | 0.4448 | 0.7114 | 0.5184 |
| (%) | (SD) | (0.15184) | (0.07550) | (0.18403) | (0.14106) | (0.05550) | (0.15427) | (0.32774) | (0.14671) |
| | Geo. | 35.0 | 14.8 | 39.2 | 45.1 | 16.1 | 40.7 | 52.0 | 28.1 |
| | CV % | | | | | | | | |

Clearance

| | | | | | | | | | |
|---|---|---|---|---|---|---|---|---|---|
| CLr | n | 6 | 3 | 5 | 6 | 6 | 6 | 7 | 6 |
| (L/h) | Mean | 1.5111 | 1.9158 | 1.6624 | 1.2032 | 1.4698 | 1.2271 | 1.5080 | 1.1463 |
| | (SD) | (0.38295) | (0.57497) | (0.61025) | (0.55907) | (0.36375) | (0.42466) | (0.55632) | (0.20889) |
| | Geo. | 26.8 | 34.8 | 37.7 | 69.6 | 23.7 | 41.9 | 42.6 | 18.9 |
| | CV % | | | | | | | | |

* λ_{z} and λ_{z-}related parameters were excluded from statistical analysis if the adjusted regression coefficient was less than 0.8 or the AUCₑₓₜᵣₐₚ exceeded 20%. Fe was calculated using 2 doses after 10 hours on Day 1. CLr = Ae_{0-10 h}/AUCₜₐᵤ, where AUCₜₐᵤ was the area under the curve for the dosing interval from 0 to 10 hours. Geometric CV % = 100 × (exp(SD²) − 1)^{0.5}, where SD was the standard deviation of the logarithmic-transformed data.

Table 6 summarizes urine Formulation A PK parameters on Day 6 for the PK Population. Urinary excretion on Day 6 followed similar patterns to that observed on Day 1 for all dosing regimens.

TABLE 6
Summary of Urine Formulation A Pharmacokinetics Parameters Day 6 - PK Population

| | | High Fat | | | Low Fat | | | Fasted | |
|---|---|---|---|---|---|---|---|---|---|
| PK | | Cohort | Cohort | Cohort | Cohort | Cohort | Cohort | Cohort | Cohort |
| Parameter | | 1a/320 | 2a/640 | 3a/1280 | 1b/320 | 2b/640 | 3b/1280 | 3c/960 | 3d/1280 |
| (Unit) | Statistic | mg | mg | mg | mg | mg | mg | mg | mg |

Amount excreted

| | | | | | | | | | |
|---|---|---|---|---|---|---|---|---|---|
| Ae (0-2 h) | n | 6 | 6 | 6 | 6 | 5 | 6 | 6 | 6 |
| (ug) | Mean | 571.3 | 874.9 | 1668.4 | 492.8 | 1034.6 | 2222.8 | 3271.1 | 4196.6 |
| | (SD) | (615.07) | (601.64) | (975.08) | (137.27) | (421.88) | (855.84) | (1395.41) | (1696.41) |
| | Geo. | 107.5 | 106.7 | 81.4 | 27.1 | 47.3 | 40.1 | 44.0 | 47.7 |
| | CV % | | | | | | | | |
| Ae (0-4 h) | n | 6 | 6 | 6 | 6 | 5 | 6 | 6 | 6 |
| (ug) | Mean | 1070.7 | 2332.9 | 3507.4 | 1011.6 | 1712.6 | 3938.7 | 5244.0 | 5895.1 |
| | (SD) | (549.75) | (430.01) | (1254.92) | (281.57) | (449.34) | (1176.59) | (1911.06) | (2489.22) |
| | Geo. | 41.9 | 18.0 | 37.3 | 30.3 | 30.3 | 36.2 | 40.4 | 59.8 |
| | CV % | | | | | | | | |
| Ae (0-6 h) | n | 6 | 6 | 6 | 6 | 5 | 6 | 6 | 6 |
| (ug) | Mean | 1311.7 | 2943.2 | 4822.5 | 1270.2 | 2190.8 | 5055.5 | 6208.4 | 7306.8 |
| | (SD) | (507.56) | (549.61) | (1709.21) | (365.67) | (546.63) | (1655.60) | (2302.33) | (1909.40) |
| | Geo. | 34.0 | 17.1 | 35.9 | 33.5 | 29.0 | 39.5 | 40.3 | 26.6 |
| | CV % | | | | | | | | |
| Ae (0- | n | 6 | 6 | 6 | 6 | 5 | 6 | 6 | 6 |
| 10 h) (ug) | Mean | 1505.9 | 3790.2 | 5850.3 | 1436.5 | 2573.1 | 5942.8 | 6747.0 | 7934.9 |
| | (SD) | (449.48) | (694.63) | (1882.51) | (460.33) | (441.42) | (1741.51) | (2565.56) | (1812.33) |
| | Geo | 27.1 | 16.6 | 33.5 | 38.6 | 18.2 | 35.7 | 41.6 | 22.3 |
| | CV % | | | | | | | | |
| Ae (0- | n | 6 | 6 | 6 | 6 | 5 | 6 | 6 | 6 |
| 12 h) (ug) | Mean | 1525.7 | 3998.1 | 6082.9 | 1455.4 | 2623.9 | 6040.0 | 6827.2 | 8023.8 |
| | (SD) | (439.39) | (705.81) | (1907.42) | (473.18) | (431.40) | (1769.39) | (2605.95) | (1828.86) |
| | Geo. | 25.9 | 16.0 | 32.0 | 39.3 | 17.5 | 35.8 | 41.8 | 22.3 |
| | CV % | | | | | | | | |
| Ae (0- | n | 6 | 6 | 6 | 6 | 5 | 6 | 6 | 6 |
| 14 h) (ug) | Mean | 1548.9 | 4073.5 | 6206.5 | 1465.4 | 2647.3 | 6095.8 | 6868.6 | 8107.8 |
| | (SD) | (439.09) | (681.50) | (1895.69) | (475.12) | (429.48) | (1784.97) | (2621.59) | (1793.67) |
| | Geo. | 25.7 | 15.1 | 31.0 | 39.2 | 17.2 | 35.7 | 41.8 | 21.5 |
| | CV % | | | | | | | | |
| Ae (0- | n | 6 | 6 | 6 | 6 | 5 | 6 | 6 | 6 |
| 16 h) (ug) | Mean | 1565.2 | 4107.3 | 6268.7 | 1470.7 | 2657.0 | 6123.8 | 6891.9 | 8143.9 |
| | (SD) | (431.75) | (664.78) | (1881.08) | (476.89) | (431.18) | (1792.40) | (2632.48) | (1792.64) |
| | Geo. | 25.2 | 14.7 | 30.4 | 39.2 | 17.2 | 35.7 | 41.8 | 21.3 |
| | CV % | | | | | | | | |
| Ae (0- | n | 6 | 6 | 6 | 6 | 5 | 6 | 6 | 6 |
| 24 h) (ug) | Mean | 1580.2 | 4147.7 | 6397.7 | 1487.5 | 2681.1 | 6188.4 | 6930.8 | 8214.4 |
| | (SD) | (431.34) | (670.39) | (1887.04) | (475.72) | (424.57) | (1801.20) | (2644.64) | (1794.30) |
| | Geo. | 25.0 | 14.7 | 29.8 | 38.7 | 16.8 | 35.3 | 41.8 | 21.1 |
| | CV % | | | | | | | | |

Fraction excreted

| | | | | | | | | | |
|---|---|---|---|---|---|---|---|---|---|
| Fe (0-2 h) | n | 6 | 6 | 6 | 6 | 5 | 6 | 6 | 6 |
| (%) | Mean | 0.1785 | 0.1367 | 0.1303 | 0.1540 | 0.1616 | 0.1737 | 0.3407 | 0.3279 |
| | (SD) | (0.19221) | (0.09401) | (0.07618) | (0.04290) | (0.06592) | (0.06686) | (0.14536) | (0.13253) |
| | Geo. | 107.5 | 106.7 | 81.4 | 27.1 | 47.3 | 40.1 | 44.0 | 47.7 |
| | CV % | | | | | | | | |
| Fe (0-4 h) | n | 6 | 6 | 6 | 6 | 5 | 6 | 6 | 6 |
| (%) | Mean | 0.3346 | 0.3645 | 0.2740 | 0.3161 | 0.2676 | 0.3077 | 0.5462 | 0.4606 |
| | (SD) | (0.17180) | (0.06719) | (0.09804) | (0.08799) | (0.07021) | (0.09192) | (0.19907) | (0.19447) |
| | Geo. | 41.9 | 18.0 | 37.3 | 30.3 | 30.3 | 36.2 | 40.4 | 59.8 |
| | CV % | | | | | | | | |
| Fe (0-6 h) | n | 6 | 6 | 6 | 6 | 5 | 6 | 6 | 6 |
| (%) | Mean | 0.4099 | 0.4599 | 0.3768 | 0.3969 | 0.3423 | 0.3950 | 0.6467 | 0.5708 |
| | (SD) | (0.15861) | (0.08588) | (0.13353) | (0.11427) | (0.08541) | (0.12934) | (0.23983) | (0.14917) |
| | Geo. | 34.0 | 17.1 | 35.9 | 33.5 | 29.0 | 39.5 | 40.3 | 26.6 |
| | CV % | | | | | | | | |
| Fe (0- | n | 6 | 6 | 6 | 6 | 5 | 6 | 6 | 6 |
| 10 h) (%) | Mean | 0.4706 | 0.5922 | 0.4571 | 0.4489 | 0.4021 | 0.4643 | 0.7028 | 0.6199 |
| | (SD) | (0.14046) | (0.10854) | (0.14707) | (0.14385) | (0.06897) | (0.13606) | (0.26725) | (0.14159) |
| | Geo. | 27.1 | 16.6 | 33.5 | 38.6 | 18.2 | 35.7 | 41.6 | 22.3 |
| | CV % | | | | | | | | |
| Fe (0- | n | 6 | 6 | 6 | 6 | 5 | 6 | 6 | 6 |
| 12 h) (%) | Mean | 0.4768 | 0.6247 | 0.4752 | 0.4548 | 0.4100 | 0.4719 | 0.7112 | 0.6269 |
| | (SD) | (0.13731) | (0.11028) | (0.14902) | (0.14787) | (0.06741) | (0.13823) | (0.27145) | (0.14288) |
| | Geo. | 25.9 | 16.0 | 32.0 | 39.3 | 17.5 | 35.8 | 41.8 | 22.3 |
| | CV % | | | | | | | | |
| Fe (0- | n | 6 | 6 | 6 | 6 | 5 | 6 | 6 | 6 |
| 14 h) (%) | Mean | 0.4840 | 0.6365 | 0.4849 | 0.4579 | 0.4136 | 0.4762 | 0.7155 | 0.6334 |
| | (SD) | (0.13722) | (0.10648) | (0.14810) | (0.14847) | (0.06711) | (0.13945) | (0.27308) | (0.14013) |
| | Geo. | 25.7 | 15.1 | 31.0 | 39.2 | 17.2 | 35.7 | 41.8 | 21.5 |
| | CV % | | | | | | | | |
| Fe (0- | n | 6 | 6 | 6 | 6 | 5 | 6 | 6 | 6 |
| 16 h) (%) | Mean | 0.4891 | 0.6418 | 0.4897 | 0.4596 | 0.4152 | 0.4784 | 0.7179 | 0.6362 |
| | (SD) | (0.13492) | (0.10387) | (0.14696) | (0.14903) | (0.06737) | (0.14003) | (0.27422) | (0.14005) |
| | Geo. | 25.2 | 14.7 | 30.4 | 39.2 | 17.2 | 35.7 | 41.8 | 21.3 |
| | CV % | | | | | | | | |
| Fe (0- | n | 6 | 6 | 6 | 6 | 5 | 6 | 6 | 6 |
| 24 h) (%) | Mean | 0.4938 | 0.6481 | 0.4998 | 0.4649 | 0.4189 | 0.4835 | 0.7220 | 0.6417 |
| | (SD) | (0.13479) | (0.10475) | (0.14742) | (0.14866) | (0.06634) | (0.14072) | (0.27548) | (0.14018) |
| | Geo. | 25.0 | 14.7 | 29.8 | 38.7 | 16.8 | 35.3 | 41.8 | 21.1 |
| | CV % | | | | | | | | |

Clearance

| | | | | | | | | | |
|---|---|---|---|---|---|---|---|---|---|
| CLr | n | 6 | 6 | 6 | 6 | 5 | 6 | 6 | 6 |
| (L/h) | Mean | 1.6736 | 2.2888 | 1.5232 | 1.5999 | 1.5795 | 1.2831 | 1.4906 | 1.2105 |
| | (SD) | (0.40152) | (0.28539) | (0.37705) | (0.55287) | (0.41112) | (0.36645) | (0.54386) | (0.36584) |
| | Geo. | 25.5 | 12.7 | 27.9 | 44.8 | 26.2 | 39.5 | 38.7 | 28.8 |
| | CV % | | | | | | | | |

* λ_{z} and λ_{z}-related parameters were excluded from statistical analysis if the adjusted regression coefficient was less than 0.8 or the AUCₑₓₜᵣₐₚ exceeded 20%. Fe was calculated using 2 doses after 10 hours on Day 1. CLr = Ae_{0-10 h}/Aucₜₐᵤ, where Aucₜₐᵤ was the area under the curve for the dosing interval from 0 to 10 hours. Geometric CV % = 100 × (exp(SD²) − 1)^{0.5}, where SD was the standard deviation of the logarithmic-transformed data.

### (iv) Pharmacokinetic and Pharmacodynamic Conclusions

Following administration of Formulation A, phloroglucinol was rapidly absorbed followed by a gradual decline. Systemic exposures to phloroglucinol generally increased with increasing dose across treatment groups; Evidence of the IR/DR/DR kinetics were observed in the current study with a slowing in the rate of decline of plasma phloroglucinol concentrations observed at approximately 2 hours after administration of Formulation A then again at approximately 4 hours after Formulation A administration. Given the overall rather rapid half-life, no noteworthy accumulation was observed at steady-state. When administered with food, the PK profile of Formulation A exhibited blunted, yet sustained exposures, irrespective of meal type. the exposures observed in this study for all dosing regimens met or exceeded those associated with therapeutic dosing regimens of Spasfon™. Mean Cₘₐₓ values following administration of 1 to 2 capsules of Formulation A were similar to those attained with 1 to 2 tablets of Spasfon™ while AUC was higher under all conditions studied. Mean Cₘₐₓ and AUC values following administration of 3 to 4 capsules of Formulation A were both well above those attained with Spasfon™ under all conditions studied.

Consistent with the rapid clearance of Formulation A, from the plasma, Formulation A was rapidly excreted in the urine. The PK profile of Formulation A in urine for both Day 1 and Day 6 generally followed similar dose-dependent trends as the corresponding plasma PK profiles for all dosing regimens studied.

An exploratory dose proportionality assessment indicated no gross trends towards a deviation from proportionality across the dose range studied.

In general, subjects typically had relatively small numbers of bowel movements per day (average of 0 to 2) throughout the entire study. Overall, the data show no clear or consistent trend in bowel movements. However, data from select cohorts and isolated subjects within various cohorts suggest that Formulation A may have produced a decrease in the number of bowel movements per day during the treatment period which returned to baseline after the treatment period completed.

### G. Safety Results

### (i) Extent of Exposure

A total of 65 subjects were randomized to 1 of 8 cohorts. Six subjects in each cohort received Formulation A dosed each morning and evening (at nominal times of 0 and 10 hours) starting on the morning of Day 1 and continuing through the morning of Day 6. The following dose levels were studied with a high or low fat meal (fed state): 320 mg, 640 mg, or 1280 mg. Two subjects in each cohort received placebo in a corresponding fed state. Six subjects received 960 mg of Formulation A and 6 subjects received 1280 mg of Formulation A on an empty stomach; and 2 subjects in each of these cohorts received placebo on an empty stomach. One additional subject commenced dosing with 960 mg Formulation A on an empty stomach but withdrew early from the study after the first dose due to concerns with being able to tolerate the number of blood draws during the treatment period.

### (ii) Adverse Events

Table 7 provides an overview of AEs for the Safety Population.

There were no TEAEs related to COVID-19. There were no treatment-emergent SAEs, study drug-related treatment-emergent SAEs, TEAEs leading to death, TEAEs leading to study discontinuation, or study drug-related TEAEs leading to study discontinuation.

Overall, 3 (25.0%), 5 (41.7%), 2 (28.6%), 6 (33.3%), and 4 (25.0%) subjects in the 320 mg, 640 mg, 960 mg, 1280 mg, and pooled placebo groups, respectively, experienced TEAEs that were either mild or moderate in severity.

Overall, 1 (8.3%), 1 (8.3%), and 2 (11.1%) subjects in the 320 mg, 640 mg, and 1280 mg Formulation A treatment groups, respectively, experienced TEAEs that were considered related to the study drug. Of these study-drug related TEAES, all were mild in severity.

No dose-dependent increases in frequency or severity of AEs were observed and there was no apparent difference in the incidence or severity of AEs based on meal conditions.

TABLE 7
Overview of Adverse Events - Safety Population

| | High Fat | | | | | | | |
|---|---|---|---|---|---|---|---|---|
| | 320 mg | | 640 mg | | 1280 mg | | Pooled Placebo | |
| | (N = 6) | | (N = 6) | | (N = 6) | | (N = 6) | |
| Category | n (%) | | n (%) | | n (%) | | n (%) | |
| Subjects with any AE | 2 | (33.3) | 4 | (66.7) | 2 | (33.3) | 2 | (33.3) |
| Subjects with any TEAE | 1 | (16.7) | 4 | (66.7) | 1 | (16.7) | 2 | (33.3) |

Subjects with any TEAE by maximum severity

| | | | | | | | | |
|---|---|---|---|---|---|---|---|---|
| Mild | 0 | (0.0) | 3 | (50.0) | 1 | (16.7) | 2 | (33.3) |
| Moderate | 1 | (16.7) | 1 | (16.7) | 0 | (0.0) | 0 | (0.0) |
| Subjects with any drug-related | 0 | (0.0) | 0 | (0.0) | 1 | (16.7) | 0 | (0.0) |
| TEAE | | | | | | | | |

Subjects with any drug-related TEAE by maximum severity

| | | | | | | | | |
|---|---|---|---|---|---|---|---|---|
| Mild | 0 | (0.0) | 0 | (0.0) | 1 | (16.7) | 0 | (0.0) |
| Moderate | 0 | (0.0) | 0 | (0.0) | 0 | (0.0) | 0 | (0.0) |
| Subjects with any TEAE related to | 0 | (0.0) | 0 | (0.0) | 0 | (0.0) | 0 | (0.0) |
| COVID-19 | | | | | | | | |
| Subjects with any | 0 | (0.0) | 0 | (0.0) | 0 | (0.0) | 0 | (0.0) |
| treatment-emergent SAE | | | | | | | | |
| Subjects with any drug-related | 0 | (0.0) | 0 | (0.0) | 0 | (0.0) | 0 | (0.0) |
| treatment-emergent SAE | | | | | | | | |
| Subjects with any TEAE leading to | 0 | (0.0) | 0 | (0.0) | 0 | (0.0) | 0 | (0.0) |
| death | | | | | | | | |
| Subjects with any TEAE leading to | 0 | (0.0) | 0 | (0.0) | 0 | (0.0) | 0 | (0.0) |
| study discontinuation | | | | | | | | |
| Subjects with any drug-related | 0 | (0.0) | 0 | (0.0) | 0 | (0.0) | 0 | (0.0) |
| TEAE leading to study | | | | | | | | |
| discontinuation | | | | | | | | |

TABLE 8
Overview of Adverse Events - Safety Population (continued)

| | Low Fat | | | | | | | |
|---|---|---|---|---|---|---|---|---|
| | 320 mg | | 640 mg | | 1280 mg | | Pooled Placebo | |
| | (N = 6) | | (N = 6) | | (N = 6) | | (N = 6) | |
| Category | n (%) | | n (%) | | n (%) | | n (%) | |
| Subjects with any AE | 2 | (33.3) | 1 | (16.7) | 4 | (66.7) | 1 | (16.7) |
| Subjects with any TEAE | 2 | (33.3) | 1 | (16.7) | 4 | (66.7) | 1 | (16.7) |

Subjects with any TEAE by maximum severity

| | | | | | | | | |
|---|---|---|---|---|---|---|---|---|
| Mild | 2 | (33.3) | 0 | (0.0) | 3 | (50.0) | 1 | (16.7) |
| Moderate | 0 | (0.0) | 1 | (16.7) | 1 | (16.7) | 0 | (0.0) |
| Subjects with any drug-related | 1 | (16.7) | 1 | (16.7) | 0 | (0.0) | 1 | (16.7) |
| TEAE | | | | | | | | |

Subjects with any drug-related TEAE by maximum severity

| | | | | | | | | |
|---|---|---|---|---|---|---|---|---|
| Mild | 1 | (16.7) | 0 | (0.0) | 0 | (0.0) | 1 | (16.7) |
| Moderate | 0 | (0.0) | 1 | (16.7) | 0 | (0.0) | 0 | (0.0) |
| Subjects with any TEAE related to | 0 | (0.0) | 0 | (0.0) | 0 | (0.0) | 0 | (0.0) |
| COVID-19 | | | | | | | | |
| Subjects with any | 0 | (0.0) | 0 | (0.0) | 0 | (0.0) | 0 | (0.0) |
| treatment-emergent SAE | | | | | | | | |
| Subjects with any drug-related | 0 | (0.0) | 0 | (0.0) | 0 | (0.0) | 0 | (0.0) |
| treatment-emergent SAE | | | | | | | | |
| Subjects with any TEAE leading to | 0 | (0.0) | 0 | (0.0) | 0 | (0.0) | 0 | (0.0) |
| death | | | | | | | | |
| Subjects with any TEAE leading to | 0 | (0.0) | 0 | (0.0) | 0 | (0.0) | 0 | (0.0) |
| study discontinuation | | | | | | | | |
| Subjects with any drug-related | 0 | (0.0) | 0 | (0.0) | 0 | (0.0) | 0 | (0.0) |
| TEAE leading to study | | | | | | | | |
| discontinuation | | | | | | | | |

TABLE 7
Overview of Adverse Events - Safety Population (continued)

| | Fasted | | | | | |
|---|---|---|---|---|---|---|
| | 960 mg | | 1280 mg | | Pooled Placebo | |
| | (N = 7) | | (N = 6) | | (N = 4) | |
| Category | n (%) | | n (%) | | n (%) | |
| Subjects with any AE | 2 | (28.6) | 1 | (16.7) | 1 | (25.0) |
| Subjects with any TEAE | 2 | (28.6) | 1 | (16.7) | 1 | (25.0) |

Subjects with any TEAE by maximum severity

| | | | | | | |
|---|---|---|---|---|---|---|
| Mild | 2 | (28.6) | 1 | (16.7) | 1 | (25.0) |
| Moderate | 0 | (0.0) | 0 | (0.0) | 0 | (0.0) |
| Subjects with any drug-related TEAE | 0 | (0.0) | 1 | (16.7) | 0 | (0.0) |

Subjects with any drug-related TEAE by maximum severity

| | | | | | | |
|---|---|---|---|---|---|---|
| Mild | 0 | (0.0) | 1 | (16.7) | 0 | (0.0) |
| Subjects with any TEAE | 0 | (0.0) | 0 | (0.0) | 0 | (0.0) |
| related to COVID-19 | | | | | | |
| Subjects with any treatment- | 0 | (0.0) | 0 | (0.0) | 0 | (0.0) |
| emergent SAE | | | | | | |
| Subjects with any drug- | 0 | (0.0) | 0 | (0.0) | 0 | (0.0) |
| related treatment-emergent SAE | | | | | | |
| Subjects with any TEAE leading to death | 0 | (0.0) | 0 | (0.0) | 0 | (0.0) |
| Subjects with any TEAE leading | 0 | (0.0) | 0 | (0.0) | 0 | (0.0) |
| to study discontinuation | | | | | | |
| Subjects with any drug-related TEAE | 0 | (0.0) | 0 | (0.0) | 0 | (0.0) |
| leading to study discontinuation | | | | | | |

% = 100 × n/N. A TEAE was defined as an AE that started after the dose of study drug. Subjects reporting more than one AE were counted only once using the most severe incident. Coding was based on MedDRA version 23.1.

TABLE 7
Overview of Adverse Events - Safety Population (continued)

| | Overall | | | | | | | | | |
|---|---|---|---|---|---|---|---|---|---|---|
| | Total | | Total | | Total | | Total | | Total Pooled | |
| | 320 mg | | 640 mg | | 960 mg | | 1280 mg | | Placebo | |
| | (N = 12) | | (N = 12) | | (N = 7) | | (N = 18) | | (N = 16) | |
| Category | n (%) | | n (%) | | n (%) | | n (%) | | n (%) | |
| Subjects with any AE | 4 | (33.3) | 5 | (41.7) | 2 | (28.6) | 7 | (38.9) | 4 | (25.0) |
| Subjects with any TEAE | 3 | (25.0) | 5 | (41.7) | 2 | (28.6) | 6 | (33.3) | 4 | (25.0) |

Subjects with any TEAE by maximum severity

| | | | | | | | | | | |
|---|---|---|---|---|---|---|---|---|---|---|
| Mild | 2 | (16.7) | 3 | (25.0) | 2 | (28.6) | 5 | (27.8) | 4 | (25.0) |
| Moderate | 1 | (8.3) | 2 | (16.7) | 0 | (0.0) | 1 | (5.6) | 0 | (0.0) |
| Subjects with any drug-related | 1 | (8.3) | 1 | (8.3) | 0 | (0.0) | 2 | (11.1) | 1 | (6.3) |
| TEAE | | | | | | | | | | |

Subjects with any drug-related TEAE by maximum severity

| | | | | | | | | | | |
|---|---|---|---|---|---|---|---|---|---|---|
| Mild | 1 | (8.3) | 0 | (0.0) | 0 | (0.0) | 2 | (11.1) | 1 | (6.3) |
| Subjects with any TEAE | 0 | (0.0) | 0 | (0.0) | 0 | (0.0) | 0 | (0.0) | 0 | (0.0) |
| related to COVID-19 | | | | | | | | | | |
| Subjects with any | 0 | (0.0) | 0 | (0.0) | 0 | (0.0) | 0 | (0.0) | 0 | (0.0) |
| treatment-emergent SAE | | | | | | | | | | |
| Subjects with any drug-related | 0 | (0.0) | 0 | (0.0) | 0 | (0.0) | 0 | (0.0) | 0 | (0.0) |
| treatment-emergent SAE | | | | | | | | | | |
| Subjects with any TEAE | 0 | (0.0) | 0 | (0.0) | 0 | (0.0) | 0 | (0.0) | 0 | (0.0) |
| leading to death | | | | | | | | | | |
| Subjects with any TEAE | 0 | (0.0) | 0 | (0.0) | 0 | (0.0) | 0 | (0.0) | 0 | (0.0) |
| leading to study | | | | | | | | | | |
| discontinuation | | | | | | | | | | |
| Subjects with any drug-related | 0 | (0.0) | 0 | (0.0) | 0 | (0.0) | 0 | (0.0) | 0 | (0.0) |
| TEAE leading to study | | | | | | | | | | |
| discontinuation | | | | | | | | | | |

% = 100 × n/N. A TEAE was defined as an AE that started after the dose of study drug. Subjects reporting more than one AE were counted only once using the most severe incident. Coding was based on MedDRA version 23.1.

Table 9 summarizes TEAEs by SOC and PT for the Safety Population. There was no apparent dose-dependent increase in the frequency of AEs with 3 (25.0%), 5 (41.7%), 2 (28.6%), 6 (33.3%), and 4 (25.0%) subjects in the 320 mg, 640 mg, 960 mg, 1280 mg, and pooled placebo groups, respectively, experiencing TEAEs. Furthermore, there was no apparent difference in the incidence or severity of AEs based on meal conditions. The most commonly reported SOCs of TEAEs were gastrointestinal disorders and nervous system disorders. There were no TEAEs related to COVID-19 during the study and no TEAEs leading to death.

TABLE 9
Treatment-Emergent Adverse Events by System Organ
Class and Preferred Term - Safety Population

| | High Fat | | | | | | | |
|---|---|---|---|---|---|---|---|---|
| | 320 mg | | 640 mg | | 1280 mg | | Pooled Placebo | |
| System Organ Class | (N = 6) | | (N = 6) | | (N = 6) | | (N = 6) | |
| Preferred Term | n (%) | | n (%) | | n (%) | | n (%) | |
| Subjects with any TEAEs | 1 | (16.7) | 4 | (66.7) | 1 | (16.7) | 2 | (33.3) |
| Gastrointestinal disorders | 0 | (0.0) | 1 | (16.7) | 1 | (16.7) | 0 | (0.0) |
| Diarrhea | 0 | (0.0) | 0 | (0.0) | 1 | (16.7) | 0 | (0.0) |
| Abdominal pain | 0 | (0.0) | 0 | (0.0) | 1 | (16.7) | 0 | (0.0) |
| Dyspepsia | 0 | (0.0) | 0 | (0.0) | 1 | (16.7) | 0 | (0.0) |
| Constipation | 0 | (0.0) | 0 | (0.0) | 0 | (0.0) | 0 | (0.0) |
| Toothache | 0 | (0.0) | 1 | (16.7) | 0 | (0.0) | 0 | (0.0) |
| Vomiting | 0 | (0.0) | 0 | (0.0) | 1 | (16.7) | 0 | (0.0) |
| Nervous system disorders | 0 | (0.0) | 1 | (16.7) | 1 | (16.7) | 0 | (0.0) |
| Headache | 0 | (0.0) | 1 | (16.7) | 1 | (16.7) | 0 | (0.0) |
| Dizziness | 0 | (0.0) | 0 | (0.0) | 0 | (0.0) | 0 | (0.0) |
| Musculoskeletal and connective | 1 | (16.7) | 0 | (0.0) | 0 | (0.0) | 1 | (16.7) |
| tissue disorders | | | | | | | | |
| Back pain | 1 | (16.7) | 0 | (0.0) | 0 | (0.0) | 1 | (16.7) |
| Myalgia | 0 | (0.0) | 0 | (0.0) | 0 | (0.0) | 0 | (0.0) |
| Infections and infestations | 0 | (0.0) | 2 | (33.3) | 0 | (0.0) | 1 | (16.7) |
| Rhinitis | 0 | (0.0) | 2 | (33.3) | 0 | (0.0) | 1 | (16.7) |
| Cardiac disorders | 0 | (0.0) | 0 | (0.0) | 0 | (0.0) | 0 | (0.0) |
| Palpitations | 0 | (0.0) | 0 | (0.0) | 0 | (0.0) | 0 | (0.0) |
| Ventricular tachycardia | 0 | (0.0) | 0 | (0.0) | 0 | (0.0) | 0 | (0.0) |
| General disorders and | 0 | (0.0) | 1 | (16.7) | 0 | (0.0) | 0 | (0.0) |
| administration site conditions | | | | | | | | |
| Fatigue | 0 | (0.0) | 0 | (0.0) | 0 | (0.0) | 0 | (0.0) |
| Pain | 0 | (0.0) | 1 | (16.7) | 0 | (0.0) | 0 | (0.0) |
| Ear and labyrinth disorders | 0 | (0.0) | 0 | (0.0) | 0 | (0.0) | 1 | (16.7) |
| Ear pain | 0 | (0.0) | 0 | (0.0) | 0 | (0.0) | 1 | (16.7) |
| Respiratory, thoracic, and | 0 | (0.0) | 1 | (16.7) | 0 | (0.0) | 0 | (0.0) |
| mediastinal disorders | | | | | | | | |
| Oropharyngeal pain | 0 | (0.0) | 1 | (16.7) | 0 | (0.0) | 0 | (0.0) |
| Vascular disorders | 0 | (0.0) | 0 | (0.0) | 0 | (0.0) | 1 | (16.7) |
| Orthostatic hypotension | 0 | (0.0) | 0 | (0.0) | 0 | (0.0) | 1 | (16.7) |

TABLE 10
Treatment-Emergent Adverse Events by System Organ Class
and Preferred Term - Safety Population (continued)

| | Low Fat | | | | | | | |
|---|---|---|---|---|---|---|---|---|
| | 320 mg | | 640 mg | | 1280 mg | | Pooled Placebo | |
| System Organ Class | (N = 6) | | (N = 6) | | (N = 6) | | (N = 6) | |
| Preferred Term | n (%) | | n (%) | | n (%) | | n (%) | |
| Subjects with any TEAEs | 2 | (33.3) | 1 | (16.7) | 4 | (66.7) | 1 | (16.7) |
| Gastrointestinal disorders | 1 | (16.7) | 0 | (0.0) | 0 | (0.0) | 1 | (16.7) |
| Diarrhea | 0 | (0.0) | 0 | (0.0) | 0 | (0.0) | 0 | (0.0) |
| Abdominal pain | 0 | (0.0) | 0 | (0.0) | 0 | (0.0) | 0 | (0.0) |
| Dyspepsia | 1 | (16.7) | 0 | (0.0) | 0 | (0.0) | 0 | (0.0) |
| Constipation | 0 | (0.0) | 0 | (0.0) | 0 | (0.0) | 1 | (16.7) |
| Toothache | 0 | (0.0) | 0 | (0.0) | 0 | (0.0) | 0 | (0.0) |
| Vomiting | 0 | (0.0) | 0 | (0.0) | 0 | (0.0) | 0 | (0.0) |
| Nervous system disorders | 1 | (16.7) | 0 | (0.0) | 2 | (33.3) | 0 | (0.0) |
| Headache | 1 | (16.7) | 0 | (0.0) | 1 | (16.7) | 0 | (0.0) |
| Dizziness | 0 | (0.0) | 0 | (0.0) | 1 | (16.7) | 0 | (0.0) |
| Musculoskeletal and connective | 1 | (16.7) | 0 | (0.0) | 2 | (33.3) | 0 | (0.0) |
| tissue disorders | | | | | | | | |
| Back pain | 0 | (0.0) | 0 | (0.0) | 2 | (33.3) | 0 | (0.0) |
| Myalgia | 1 | (16.7) | 0 | (0.0) | 0 | (0.0) | 0 | (0.0) |
| Infections and infestations | 0 | (0.0) | 0 | (0.0) | 0 | (0.0) | 0 | (0.0) |
| Rhinitis | 0 | (0.0) | 0 | (0.0) | 0 | (0.0) | 0 | (0.0) |
| Cardiac disorders | 1 | (16.7) | 1 | (16.7) | 0 | (0.0) | 0 | (0.0) |
| Palpitations | 1 | (16.7) | 0 | (0.0) | 0 | (0.0) | 0 | (0.0) |
| Ventricular tachycardia | 0 | (0.0) | 1 | (16.7) | 0 | (0.0) | 0 | (0.0) |
| General disorders and | 1 | (16.7) | 0 | (0.0) | 0 | (0.0) | 0 | (0.0) |
| administration site conditions | | | | | | | | |
| Fatigue | 1 | (16.7) | 0 | (0.0) | 0 | (0.0) | 0 | (0.0) |
| Pain | 0 | (0.0) | 0 | (0.0) | 0 | (0.0) | 0 | (0.0) |
| Ear and labyrinth disorders | 0 | (0.0) | 0 | (0.0) | 0 | (0.0) | 0 | (0.0) |
| Ear pain | 0 | (0.0) | 0 | (0.0) | 0 | (0.0) | 0 | (0.0) |
| Respiratory, thoracic, and | 0 | (0.0) | 0 | (0.0) | 0 | (0.0) | 0 | (0.0) |
| mediastinal disorders | | | | | | | | |
| Oropharyngeal pain | 0 | (0.0) | 0 | (0.0) | 0 | (0.0) | 0 | (0.0) |
| Vascular disorders | 0 | (0.0) | 0 | (0.0) | 0 | (0.0) | 0 | (0.0) |
| Orthostatic hypotension | 0 | (0.0) | 0 | (0.0) | 0 | (0.0) | 0 | (0.0) |

TABLE 9
Treatment-Emergent Adverse Events by System Organ Class
and Preferred Term - Safety Population (continued)

| | Fasted | | | | | |
|---|---|---|---|---|---|---|
| | 960 mg | | 1280 mg | | Pooled Placebo | |
| System Organ Class | (N = 7) | | (N = 6) | | (N = 4) | |
| Preferred Term | n (%) | | n (%) | | n (%) | |
| Subjects with any TEAEs | 2 | (28.6) | 1 | (16.7) | 1 | (25.0) |
| Gastrointestinal disorders | 1 | (14.3) | 1 | (16.7) | 0 | (0.0) |
| Diarrhea | 1 | (14.3) | 1 | (16.7) | 0 | (0.0) |
| Abdominal pain | 0 | (0.0) | 1 | (16.7) | 0 | (0.0) |
| Dyspepsia | 0 | (0.0) | 0 | (0.0) | 0 | (0.0) |
| Constipation | 0 | (0.0) | 0 | (0.0) | 0 | (0.0) |
| Flatulence | 0 | (0.0) | 1 | (16.7) | 0 | (0.0) |
| Toothache | 0 | (0.0) | 0 | (0.0) | 0 | (0.0) |
| Vomiting | 0 | (0.0) | 0 | (0.0) | 0 | (0.0) |
| Nervous system disorders | 1 | (14.3) | 0 | (0.0) | 0 | (0.0) |
| Headache | 1 | (14.3) | 0 | (0.0) | 0 | (0.0) |
| Dizziness | 0 | (0.0) | 0 | (0.0) | 0 | (0.0) |
| Musculoskeletal and connective | 0 | (0.0) | 0 | (0.0) | 0 | (0.0) |
| tissue disorders | | | | | | |
| Back pain | 0 | (0.0) | 0 | (0.0) | 0 | (0.0) |
| Myalgia | 0 | (0.0) | 0 | (0.0) | 0 | (0.0) |
| Infections and infestations | 0 | (0.0) | 0 | (0.0) | 0 | (0.0) |
| Rhinitis | 0 | (0.0) | 0 | (0.0) | 0 | (0.0) |
| Cardiac disorders | 0 | (0.0) | 0 | (0.0) | 0 | (0.0) |
| Palpitations | 0 | (0.0) | 0 | (0.0) | 0 | (0.0) |
| Ventricular tachycardia | 0 | (0.0) | 0 | (0.0) | 0 | (0.0) |
| General disorders and administration | 0 | (0.0) | 0 | (0.0) | 0 | (0.0) |
| site conditions | | | | | | |
| Fatigue | 0 | (0.0) | 0 | (0.0) | 0 | (0.0) |
| Pain | 0 | (0.0) | 0 | (0.0) | 0 | (0.0) |
| Ear and labyrinth disorders | 0 | (0.0) | 0 | (0.0) | 0 | (0.0) |
| Ear pain | 0 | (0.0) | 0 | (0.0) | 0 | (0.0) |
| Respiratory, thoracic, and | 0 | (0.0) | 0 | (0.0) | 0 | (0.0) |
| mediastinal disorders | | | | | | |
| Oropharyngeal pain | 0 | (0.0) | 0 | (0.0) | 0 | (0.0) |
| Skin and subcutaneous tissue | 0 | (0.0) | 0 | (0.0) | 1 | (25.0) |
| disorders | | | | | | |
| Dermatitis contact | 0 | (0.0) | 0 | (0.0) | 1 | (25.0) |
| Vascular disorders | 0 | (0.0) | 0 | (0.0) | 0 | (0.0) |
| Orthostatic hypotension | 0 | (0.0) | 0 | (0.0) | 0 | (0.0) |

| | Overall | | | | | | | | | |
|---|---|---|---|---|---|---|---|---|---|---|
| | | | | | | | | | Total | |
| | Total | | Total | | Total | | Total | | Pooled | |
| | 320 mg | | 640 mg | | 960 mg | | 1280 mg | | Placebo | |
| System Organ Class | (N = 12) | | (N = 12) | | (N = 7) | | (N = 18) | | (N = 16) | |
| Preferred Term | n (%) | | n (%) | | n (%) | | n (%) | | n (%) | |
| Subjects with any TEAEs | 3 | (25.0) | 5 | (41.7) | 2 | (28.6) | 6 | (33.3) | 4 | (25.0) |
| Gastrointestinal disorders | 1 | (8.3) | 1 | (8.3) | 1 | (14.3) | 2 | (11.1) | 1 | (6.3) |
| Diarrhea | 0 | (0.0) | 0 | (0.0) | 1 | (14.3) | 2 | (11.1) | 0 | (0.0) |
| Abdominal pain | 0 | (0.0) | 0 | (0.0) | 0 | (0.0) | 2 | (11.1) | 0 | (0.0) |
| Dyspepsia | 1 | (8.3) | 0 | (0.0) | 0 | (0.0) | 1 | (5.6) | 0 | (0.0) |
| Constipation | 0 | (0.0) | 0 | (0.0) | 0 | (0.0) | 0 | (0.0) | 1 | (6.3) |
| Flatulence | 0 | (0.0) | 0 | (0.0) | 0 | (0.0) | 1 | (5.6) | 0 | (0.0) |
| Toothache | 0 | (0.0) | 1 | (8.3) | 0 | (0.0) | 0 | (0.0) | 0 | (0.0) |
| Vomiting | 0 | (0.0) | 0 | (0.0) | 0 | (0.0) | 1 | (5.6) | 0 | (0.0) |
| Nervous system disorders | 1 | (8.3) | 1 | (8.3) | 1 | (14.3) | 3 | (16.7) | 0 | (0.0) |
| Headache | 1 | (8.3) | 1 | (8.3) | 1 | (14.3) | 2 | (11.1) | 0 | (0.0) |
| Dizziness | 0 | (0.0) | 0 | (0.0) | 0 | (0.0) | 1 | (5.6) | 0 | (0.0) |
| Musculoskeletal and | 2 | (16.7) | 0 | (0.0) | 0 | (0.0) | 2 | (11.1) | 1 | (6.3) |
| connective tissue disorders | | | | | | | | | | |
| Back pain | 1 | (8.3) | 0 | (0.0) | 0 | (0.0) | 2 | (11.1) | 1 | (6.3) |
| Myalgia | 1 | (8.3) | 0 | (0.0) | 0 | (0.0) | 0 | (0.0) | 0 | (0.0) |
| Infections and infestations | 0 | (0.0) | 2 | (16.7) | 0 | (0.0) | 0 | (0.0) | 1 | (6.3) |
| Rhinitis | 0 | (0.0) | 2 | (16.7) | 0 | (0.0) | 0 | (0.0) | 1 | (6.3) |
| Cardiac disorders | 1 | (8.3) | 1 | (8.3) | 0 | (0.0) | 0 | (0.0) | 0 | (0.0) |
| Palpitations | 1 | (8.3) | 0 | (0.0) | 0 | (0.0) | 0 | (0.0) | 0 | (0.0) |
| Ventricular tachycardia | 0 | (0.0) | 1 | (8.3) | 0 | (0.0) | 0 | (0.0) | 0 | (0.0) |
| General disorders and | 1 | (8.3) | 1 | (8.3) | 0 | (0.0) | 0 | (0.0) | 0 | (0.0) |
| administration site conditions | | | | | | | | | | |
| Fatigue | 1 | (8.3) | 0 | (0.0) | 0 | (0.0) | 0 | (0.0) | 0 | (0.0) |
| Pain | 0 | (0.0) | 1 | (8.3) | 0 | (0.0) | 0 | (0.0) | 0 | (0.0) |
| Ear and labyrinth disorders | 0 | (0.0) | 0 | (0.0) | 0 | (0.0) | 0 | (0.0) | 1 | (6.3) |
| Ear pain | 0 | (0.0) | 0 | (0.0) | 0 | (0.0) | 0 | (0.0) | 1 | (6.3) |
| Respiratory, thoracic, and | 0 | (0.0) | 1 | (8.3) | 0 | (0.0) | 0 | (0.0) | 0 | (0.0) |
| mediastinal disorders | | | | | | | | | | |
| Oropharyngeal pain | 0 | (0.0) | 1 | (8.3) | 0 | (0.0) | 0 | (0.0) | 0 | (0.0) |
| Skin and subcutaneous tissue | 0 | (0.0) | 0 | (0.0) | 0 | (0.0) | 0 | (0.0) | 1 | (6.3) |
| disorders | | | | | | | | | | |
| Dermatitis contact | 0 | (0.0) | 0 | (0.0) | 0 | (0.0) | 0 | (0.0) | 1 | (6.3) |
| Vascular disorders | 0 | (0.0) | 0 | (0.0) | 0 | (0.0) | 0 | (0.0) | 1 | (6.3) |
| Orthostatic hypotension | 0 | (0.0) | 0 | (0.0) | 0 | (0.0) | 0 | (0.0) | 1 | (6.3) |

% = 100 × n/N. A TEAE was defined as an AE that started after the dose of study drug. Subjects reporting more than 1 AE for a given MedDRA preferred term were counted only once for that term. Subjects reporting more than 1 type of event within a SOC were only counted once for that SOC.

### Drug-Related Adverse Events

Error! Reference source not found. displays study drug-related TEAEs by SOC and PT for the Safety Population. Overall, 1 (8.3%), 1 (8.3%), 2 (11.1%), and 1 (6.3%) subjects in the 320 mg, 640 mg, 1280 mg, and pooled placebo groups, respectively, experienced drug-related TEAEs. In the overall Formulation A 320 mg treatment group, 1 (8.3%) subject experienced dyspepsia, fatigue, myalgia, and headache. In the overall Formulation A 640 mg treatment group, 1 (8.3%) subject experienced ventricular tachycardia. In the overall Formulation A 1280 mg treatment group, 2 (11.1%) subjects experienced abdominal pain and diarrhea. One (5.6%) of these subjects also experienced dyspepsia and flatulence. In the pooled placebo group, 1 (6.3%) subject experienced constipation . . .

TABLE 11
Study Drug-Related Treatment-Emergent Adverse Events by
System Organ Class and Preferred Term - Safety Population

| | High Fat | | | |
|---|---|---|---|---|
| | 320 mg | 640 mg | 1280 mg | Pooled Placebo |
| System Organ Class | (N = 6) | (N = 6) | (N = 6) | (N = 6) |
| Preferred Term | n (%) | n (%) | n (%) | n (%) |
| Subjects with any drug-related | 0 (0.0) | 0 (0.0) | 1 (16.7) | 0 (0.0) |
| TEAEs | | | | |
| Gastrointestinal disorders | 0 (0.0) | 0 (0.0) | 1 (16.7) | 0 (0.0) |
| Abdominal pain | 0 (0.0) | 0 (0.0) | 1 (16.7) | 0 (0.0) |
| Diarrhea | 0 (0.0) | 0 (0.0) | 1 (16.7) | 0 (0.0) |
| Dyspepsia | 0 (0.0) | 0 (0.0) | 1 (16.7) | 0 (0.0) |
| Constipation | 0 (0.0) | 0 (0.0) | 0 (0.0) | 0 (0.0) |
| Cardiac disorders | 0 (0.0) | 0 (0.0) | 0 (0.0) | 0 (0.0) |
| Ventricular tachycardia | 0 (0.0) | 0 (0.0) | 0 (0.0) | 0 (0.0) |
| General disorders and | 0 (0.0) | 0 (0.0) | 0 (0.0) | 0 (0.0) |
| administration site conditions | | | | |
| Fatigue | 0 (0.0) | 0 (0.0) | 0 (0.0) | 0 (0.0) |
| Musculoskeletal and connective | 0 (0.0) | 0 (0.0) | 0 (0.0) | 0 (0.0) |
| tissue disorders | | | | |
| Myalgia | 0 (0.0) | 0 (0.0) | 0 (0.0) | 0 (0.0) |
| Nervous system disorders | 0 (0.0) | 0 (0.0) | 0 (0.0) | 0 (0.0) |
| Headache | 0 (0.0) | 0 (0.0) | 0 (0.0) | 0 (0.0) |

Error! Reference source not found.. Study Drug-Related
Treatment-Emergent Adverse Events by System 7Organ
Class and Preferred Term - Safety Population (continued)

| | Low Fat | | | |
|---|---|---|---|---|
| | 320 mg | 640 mg | 1280 mg | Pooled Placebo |
| System Organ Class | (N = 6) | (N = 6) | (N = 6) | (N = 6) |
| Preferred Term | n (%) | n (%) | n (%) | n (%) |
| Subjects with any drug-related | 1 (16.7) | 1 (16.7) | 0 (0.0) | 1 (16.7) |
| TEAEs | | | | |
| Gastrointestinal disorders | 1 (16.7) | 0 (0.0) | 0 (0.0) | 1 (16.7) |
| Abdominal pain | 0 (0.0) | 0 (0.0) | 0 (0.0) | 0 (0.0) |
| Diarrhea | 0 (0.0) | 0 (0.0) | 0 (0.0) | 0 (0.0) |
| Dyspepsia | 1 (16.7) | 0 (0.0) | 0 (0.0) | 0 (0.0) |
| Constipation | 0 (0.0) | 0 (0.0) | 0 (0.0) | 1 (16.7) |
| Cardiac disorders | 0 (0.0) | 1 (16.7) | 0 (0.0) | 0 (0.0) |
| Ventricular tachycardia | 0 (0.0) | 1 (16.7) | 0 (0.0) | 0 (0.0) |
| General disorders and | 1 (16.7) | 0 (0.0) | 0 (0.0) | 0 (0.0) |
| administration site conditions | | | | |
| Fatigue | 1 (16.7) | 0 (0.0) | 0 (0.0) | 0 (0.0) |
| Musculoskeletal and connective | 1 (16.7) | 0 (0.0) | 0 (0.0) | 0 (0.0) |
| tissue disorders | | | | |
| Myalgia | 1 (16.7) | 0 (0.0) | 0 (0.0) | 0 (0.0) |
| Nervous system disorders | 1 (16.7) | 0 (0.0) | 0 (0.0) | 0 (0.0) |
| Headache | 1 (16.7) | 0 (0.0) | 0 (0.0) | 0 (0.0) |

Error! Reference source not found.. Study Drug-Related
Treatment-Emergent Adverse Events by System Organ
Class and Preferred Term - Safety Population (continued)

| | Fasted | | |
|---|---|---|---|
| | 960 mg | 1280 mg | Pooled Placebo |
| System Organ Class | (N = 7) | (N = 6) | (N = 4) |
| Preferred Term | n (%) | n (%) | n (%) |
| Subjects with any drug-related | 0 (0.0) | 1 (16.7) | 0 (0.0) |
| TEAEs | | | |
| Gastrointestinal disorders | 0 (0.0) | 1 (16.7) | 0 (0.0) |
| Abdominal pain | 0 (0.0) | 1 (16.7) | 0 (0.0) |
| Diarrhea | 0 (0.0) | 1 (16.7) | 0 (0.0) |
| Dyspepsia | 0 (0.0) | 0 (0.0) | 0 (0.0) |
| Constipation | 0 (0.0) | 0 (0.0) | 0 (0.0) |
| Flatulence | 0 (0.0) | 1 (16.7) | 0 (0.0) |
| Cardiac disorders | 0 (0.0) | 0 (0.0) | 0 (0.0) |
| Ventricular tachycardia | 0 (0.0) | 0 (0.0) | 0 (0.0) |
| General disorders and | 0 (0.0) | 0 (0.0) | 0 (0.0) |
| administration site conditions | | | |
| Fatigue | 0 (0.0) | 0 (0.0) | 0 (0.0) |
| Musculoskeletal and | 0 (0.0) | 0 (0.0) | 0 (0.0) |
| connective tissue disorders | | | |
| Myalgia | 0 (0.0) | 0 (0.0) | 0 (0.0) |
| Nervous system disorders | 0 (0.0) | 0 (0.0) | 0 (0.0) |
| Headache | 0 (0.0) | 0 (0.0) | 0 (0.0) |

Error! Reference source not found.. Study Drug-Related
Treatment-Emergent Adverse Events by System Organ
Class and Preferred Term - Safety Population (continued)

| | | | | Overall | | | | |
|---|---|---|---|---|---|---|---|---|
| | Fasted | | | | | | | Total |
| System Organ | | | Pooled | Total | Total | Total | Total | Pooled |
| Class | 960 mg | 1280 mg | Placebo | 320 mg | 640 mg | 960 mg | 1280 mg | Placebo |
| Preferred | (N = 7) | (N = 6) | (N = 4) | (N = 12) | (N = 12) | (N = 7) | (N = 18) | (N = 16) |
| Term | n (%) | n (%) | n (%) | n (%) | n (%) | n (%) | n (%) | n (%) |
| Subjects with | 0 | 1 | 0 | 1 | 1 | 0 | 2 | 1 |
| any drug- | (0.0) | (16.7) | (0.0) | (8.3) | (8.3) | (0.0) | (11.1) | (6.3) |
| related TEAEs | | | | | | | | |
| Gastrointestinal | 0 | 1 | 0 | 1 | 0 | 0 | 2 | 1 |
| disorders | (0.0) | (16.7) | (0.0) | (8.3) | (0.0) | (0.0) | (11.1) | (6.3) |
| Abdominal | 0 | 1 | 0 | 0 | 0 | 0 | 2 | 0 |
| pain | (0.0) | (16.7) | (0.0) | (0.0) | (0.0) | (0.0) | (11.1) | (0.0) |
| Diarrhea | 0 | 1 | 0 | 0 | 0 | 0 | 2 | 0 |
| | (0.0) | (16.7) | (0.0) | (0.0) | (0.0) | (0.0) | (11.1) | (0.0) |
| Dyspepsia | 0 | 0 | 0 | 1 | 0 | 0 | 1 | 0 |
| | (0.0) | (0.0) | (0.0) | (8.3) | (0.0) | (0.0) | (5.6) | (0.0) |
| Constipation | 0 | 0 | 0 | 0 | 0 | 0 | 0 | 1 |
| | (0.0) | (0.0) | (0.0) | (0.0) | (0.0) | (0.0) | (0.0) | (6.3) |
| Flatulence | 0 | 1 | 0 | 0 | 0 | 0 | 1 | 0 |
| | (0.0) | (16.7) | (0.0) | (0.0) | (0.0) | (0.0) | (5.6) | (0.0) |
| Cardiac | 0 | 0 | 0 | 0 | 1 | 0 | 0 | 0 |
| disorders | (0.0) | (0.0) | (0.0) | (0.0) | (8.3) | (0.0) | (0.0) | (0.0) |
| Ventricular | 0 | 0 | 0 | 0 | 1 | 0 | 0 | 0 |
| tachycardia | (0.0) | (0.0) | (0.0) | (0.0) | (8.3) | (0.0) | (0.0) | (0.0) |
| General | 0 | 0 | 0 | 1 | 0 | 0 | 0 | 0 |
| disorders and | (0.0) | (0.0) | (0.0) | (8.3) | (0.0) | (0.0) | (0.0) | (0.0) |
| administration | | | | | | | | |
| site conditions | | | | | | | | |
| Fatigue | 0 | 0 | 0 | 1 | 0 | 0 | 0 | 0 |
| | (0.0) | (0.0) | (0.0) | (8.3) | (0.0) | (0.0) | (0.0) | (0.0) |
| Musculoskeletal | 0 | 0 | 0 | 1 | 0 | 0 | 0 | 0 |
| and connective | (0.0) | (0.0) | (0.0) | (8.3) | (0.0) | (0.0) | (0.0) | (0.0) |
| tissue disorders | | | | | | | | |
| Myalgia | 0 | 0 | 0 | 1 | 0 | 0 | 0 | 0 |
| | (0.0) | (0.0) | (0.0) | (8.3) | (0.0) | (0.0) | (0.0) | (0.0) |
| Nervous system | 0 | 0 | 0 | 1 | 0 | 0 | 0 | 0 |
| disorders | (0.0) | (0.0) | (0.0) | (8.3) | (0.0) | (0.0) | (0.0) | (0.0) |
| Headache | 0 | 0 | 0 | 1 | 0 | 0 | 0 | 0 |
| | (0.0) | (0.0) | (0.0) | (8.3) | (0.0) | (0.0) | (0.0) | (0.0) |

% = 100 × n/N. A TEAE was defined as an AE that started after the dose of study drug. Subjects reporting more than 1 AE for a given MedDRA preferred term were counted only once for that term. Subjects reporting more than 1 type of event within a SOC were only counted once for that SOC.

### H. Safety Conclusions

The results of this study demonstrated that Formulation A was safe and well tolerated in healthy subjects at oral doses ranging from 320 mg to 1280 mg administered each morning and evening (at nominal times of 0 and 10 hours) starting on the morning of Day 1 and continuing through the morning of Day 6. No subjects died, experienced a treatment-emergent SAE, or discontinued from the study due to a TEAE. The incidence of TEAEs was generally comparable between subjects receiving Formulation A (25.0%, 41.7%, 28.6%, and 33.3% of subjects in the 320 mg, 640 mg, 960 mg, and 1280 mg Formulation A treatment groups, respectively), and subjects receiving placebo (25.0% of placebo subjects). All TEAEs were mild or moderate in severity. No dose-dependent increases in frequency or severity of AEs were observed and there was no apparent difference in the incidence or severity of AEs based on meal conditions.

There were no meaningful changes from baseline in laboratory parameters, vital signs, 12-lead ECG findings, or skin and mucosa evaluations during the study.

No dose-dependent increases in frequency or severity of AEs were observed and there was no apparent difference in incidence or severity of AEs based on meal conditions. There were no meaningful changes from baseline in laboratory parameters, vital signs, 12-lead ECG findings, or skin and mucosa evaluations during the study.

### I. Overall Conclusions

The following conclusions can be drawn based on this study:
Formulation A was safe and well tolerated in healthy subjects at single oral doses ranging from 320 mg to 1280 mg administered each morning and evening (at nominal times of 0 and 10 hours) starting on the morning of Day 1 and continuing through the morning of Day 6 under all conditions studied, including the fasted state which produced a rapid increase to high exposures;
Evidence of IR/DR/DR kinetics was observed following the administration of Formulation A with no noteworthy accumulation following dosing each morning and evening; and
In the presence of food, the PK profile of Formulation A exhibited blunted, yet sustained exposures, irrespective of meal type.

The embodiments shown and described in the specification are only specific embodiments of inventors who are skilled in the art and are not limiting in any way. Therefore, various changes, modifications, or alterations to those embodiments may be made without departing from the spirit of the invention in the scope of the following claims. The references cited are expressly incorporated by reference herein in their entirety.

1. A pharmaceutical composition comprising a total amount of about 50 mg to about 1000 mg of an active pharmaceutical ingredient (API) that is phloroglucinol, trimethylphloroglucinol, a pharmaceutically acceptable salt thereof, or a combination thereof, wherein the pharmaceutical composition comprises:
an immediate release portion comprising about 20 to about 40% by weight of the total amount of the API, wherein at least about 90% by weight of the API in the immediate release portion is released from the pharmaceutical composition within about 2 hours, as measured by the USP 2 paddle method at about 50 rpm in about 750 mL of an aqueous solution comprising about 0.1N HCl solution at about 37° C.;
a first modified release portion comprising about 20 to about 40% by weight of the total amount of the API, wherein at least about 90% by weight of the API in the first modified release portion is released from the pharmaceutical composition after at least about 2 hours to about 4 hours, as measured by the USP 2 paddle method at about 50 rpm in about 750 mL of an aqueous solution comprising about 0.1N HCl solution at about 37° C.; and
a second modified release portion comprising about 20 to about 40% by weight of the total amount of the API, wherein at least about 90% by weight of the API in the second modified release portion is released from the pharmaceutical composition after about 4 or more hours, as measured by the USP 2 paddle method at about 50 rpm in about 750 mL of an aqueous solution comprising about 0.1N HCl solution at about 37° C.

2. The pharmaceutical composition of claim 1, wherein the API is phloroglucinol, trimethylphloroglucinol, or a pharmaceutically acceptable salt thereof.

3. (canceled)

4. The pharmaceutical composition of claim 1, in the form of a plurality of beads or granules

5. (canceled)

6. The pharmaceutical composition of claim 1, wherein the first modified release portion comprises an API-containing core coated with a first enteric polymer capable of dissolution at a pH of about 5.5.

7. The pharmaceutical composition of claim 6, wherein the first enteric polymer is a methacrylic acid copolymer, such as a methacrylic acid ethyl acrylate copolymer, or such as a 1:1 methacrylic acid:ethyl acrylate copolymer, or such as a Eudragit® L 30 D-55 polymer.

8. (canceled)

9. The pharmaceutical composition of claim 1, wherein the second modified release portion comprises an API-containing core coated with a second enteric polymer capable of dissolution at a pH of 6.8 or higher.

10. The pharmaceutical composition of claim 9, wherein the enteric polymer is a methacrylic acid copolymer, such as a methacrylic acid, methyl acrylate, methyl methacrylate copolymer, or such as a Eudragit® FS 30D copolymer.

11. (canceled)

12. (canceled)

13. The pharmaceutical composition of claim 1, wherein;
the immediate release portion is coated onto the first modified release portion, or
the immediate release portion is coated onto the second modified release portion, or
the immediate release portion is coated onto a combination of the first release portion and the second release portion.

14. The pharmaceutical composition of claim 1, wherein the first modified release portion is coated onto the second release portion.

15. The pharmaceutical composition of claim 14, wherein the immediate release portion is coated onto the first modified release portion.

16. The pharmaceutical composition of claim 1, wherein at least about 30% by weight of the total amount of API in the composition is released from the composition over a period of about 5 minutes to about 2 hours as measured by the USP 2 paddle method at about 50 rpm in about 750 mL of an aqueous solution comprising about 0.1N HCl solution at about 37° C.

17. The pharmaceutical composition of claim 1, wherein at least about 60% by weight of the total amount of the API in the composition is released from the pharmaceutical composition over a period of about 2 hours to about 4 hours as measured by the USP 2 paddle method at about 50 rpm in about 750 mL of an aqueous solution comprising about 0.1N HCl solution at about 37° C.

18. The pharmaceutical composition of claim 1, wherein at least about 80% by weight of the total amount of the API in the composition is released from the pharmaceutical composition over a period of about 3 hours to about 6 hours as measured by the USP 2 paddle method at about 50 rpm in about 750 mL of an aqueous solution comprising about 0.1N HCl solution at about 37° C.

19. The pharmaceutical composition of claim 1, comprising:
about 50 mg to about 500 mg of the API in the immediate release portion, such as about 50 mg to about 400 mg, or such as about 160 mg.

20. The pharmaceutical composition of claim 1, comprising about 25 mg to about 200 mg of the API in the first modified release portion, such as about 50 mg to about 200 mg, or such as about 160 mg, or such as about 80 mg.

21. The pharmaceutical composition of claim 1, comprising about 25 mg to about 300 mg of the API in the second modified release portion, such as about 25 mg to about 200 mg, or such as about 160 mg, or such as about 80 mg.

22. (canceled)

23. An oral dosage unit comprising the pharmaceutical composition of claim 1.

24. The oral dosage unit of claim 23, comprising:
about 10 to about 60% by weight, based on the weight of the oral dosage unit, of the immediate release portion, or such as about 30 to about 50% by weight, or such as about 43% by weight; and/or
about 10 to about 40% by weight, based on the weight of the oral dosage unit, of the first modified release portion, or such as about 20 to about 30% by weight, or such as about 28% by weight; and/or
about 10 to about 40% by weight, based on the weight of the oral dosage unit, of the second modified release portion, or such as about 20 to about 30% by weight, or such as about 28% by weight.

25-27. (canceled)

28. A method of treating a spasmodic condition in a subject in need thereof, comprising orally administering to the subject, an oral dosage unit of claim 23, wherein the oral administration to the subject in a fed state results in
a median Tₘₐₓ of the API of about 0.5 to about 1.75 hours;
a mean Cₘₐₓ of the API of about 269 to about 1512 ng/ml;
a mean AUCₜₐᵤ of the API of about 879 to about 4695 h*ng/ml; and/or
a mean t½ of the API of about 1.6 hours to about 2.4 hours
or
wherein the oral administration to the subject in a fasted state results in
a median Tₘₐₓ of the API of about 0.5 hours;
a mean Cₘₐₓ of the API of about 2745 to about 4874 ng/mL;
a mean AUCₜₐᵤ of the API of about 4567 to about 6853 h*ng/mL; and/or
a mean t½ of the API of about 2 hours.

29. The method of claim 28, wherein the spasmodic condition is a sudden involuntary muscle contraction of the bronchi, stomach, intestine, ureter, gall bladder, kidney, or bile duct; gallstones; a gastrointestinal disorder; inflammatory bowel syndrome; or renal colicky pain.

30-39. (canceled)
